SEQUENCE LISTING (40 sequences)

SEQ ID NO: 1 - DNA, 879 bp, Vigna angularis
atgggttcttctttgtggacttgtctgattctgttatcactggcttctgcttctttcgctgccaacccaagaactccaattgatgtaccatttggcagaaactatgtgcctacttgggcctttgatcatatcaaatatctcaatggaggttctgagattcagcttcatctcgataagtacactggtactggattccagtccaaagggtcatacttgtttggtcacttcagcatgtacataaaattggttcctggtgattcagctggcacagtcactgctttctatttatcgtccacaaacgcagaacatgatgaaatagacttcgagttcttgggaaacagaactgggcaaccatacattttacaaacaaatgtgttcaccggaggcaaaggtgacagagagcagagaatctacctctggtttgaccctacgactcaataccacagatattcagtgctatggaacatgtaccagattgtattctatgtggatgactacccaataagggtgttcaagaacagcaatgacttgggagtgaagttccccttcaatcaaccaatgaaaatatacaacagtttgtggaatgcagatgactgggctacaaggggtggtttggagaaaacagattggtccaaagcccccttcatagcctcttacaagggcttccacattgatgggtgtgaggcctcagtgaatgccaagttctgtgacacacaaggcaagaggtggtgggatcaaccagagtttcgtgaccttgatgctgctcagtggcaaaaactggcttgggtacgcaacaaatacaccatctacaactactgcactgatcgcaaacgctactctcaagtccctccagagtgcaccagagaccgtgacatttaa

SEQ ID NO: 2 - DNA, 876 bp, Vigna angularis
atgggctcgtccctctggacttgtttgatcctcctctccttggcatcggcatccttcgcagcgaaccctcgaactccgatcgatgtgcctttcggacggaactacgtgccgacatgggcattcgaccacattaagtatttgaacggaggctcggagatccagttgcatctcgacaagtacaccggcactggtttccagtcgaagggctcctacttgttcggacatttctccatgtacatcaaattggtgcctggtgactcggcaggaactgtcaccgcattctacctctcgtcgacaaacgcagagcatgacgaaatcgacttcgagttcctcggcaacaggacaggacagccgtacatcctccagaccaacgtcttcacaggaggcaaaggtgatcgggaacagcggatctacttgtggttcgatcccacaacccagtaccataggtactcggtgctctggaacatgtatcagatcgtcttctacgtcgacgattatccgatccgagtgttcaagaactccaacgacttgggcgtcaaattccccttcaaccagcccatgaagatttacaactcgttgtggaacgccgacgattgggcaaccaggggtggtctcgagaagacagattggtcgaaagcacctttcatcgcgtcgtacaagggtttccacatcgacggatgtgaagcctccgtgaacgccaagttctgtgacacccagggcaaacgatggtgggatcagccggaattccgggatttggatgcagcccagtggcagaagctcgcgtgggtcaggaacaagtacaccatctataactactgtaccgatcggaaacgatattcgcaggtgcctcccgagtgtacacgcgatagggacatc

SEQ ID NO: 3 - protein, 292 aa, Vigna angularis
MGSSLWTCLILLSLASASFAANPRTPIDVPFGRNYVPTWAFDHIKYLNGGSEIQLHLDKYTGTGFQSKGSYLFGHFSMYIKLVPGDSAGTVTAFYLSSTNAEHDEIDFEFLGNRTGQPYILQTNVFTGGKGDREQRIYLWFDPTTQYHRYSVLWNMYQIVFYVDDYPIRVFKNSNDLGVKFPFNQPMKIYNSLWNADDWATRGGLEKTDWSKAPFIASYKGFHIDGCEASVNAKFCDTQGKRWWDQPEFRDLDAAQWQKLAWVRNKYTIYNYCTDRKRYSQVPPECTRDRDI

SEQ ID NO: 4 - DNA, 864 bp, Arabidopsis thaliana
atggcgtgtttcgcaaccaaacagcctctgttgttgtctctcctccttgccattggcttctttgtggtggctgcatctgccggaaacttctatgagagctttgatatcacttggggtaatggtcgtgccaacatattcgagaatggacagcttctcacttgtactcttgacaaggtctccggctcaggttttcaatccaagaaggagtacttgtttggtaagatcgacatgaagctcaagcttgtcgctggaaactctgctggcaccgtcaccgcctactacctatcgtcaaaaggcacggcatgggatgagattgacttcgagtttttgggaaatcgcacaggacatccttacactatccacaccaatgtgttcaccggaggtaaaggcgaccgtgagatgcagttccgtctctggttcgatcccactgcggatttccacacctacaccgtccactggaaccctgttaacatcatcttccttgtggatgggatcccaattcgggtgttcaagaacaacgagaaaaatggggtggcttaccctaagaaccagccgatgaggatatactcaagcctttgggaagccgatgactgggctacagaaggcggtcgcgtgaagatcgactggagcaacgcaccattcaaggcctcttacagaaacttcaacgaccaaagctcatgcagcaggacatcaagctcaaaatgggtgacttgcgagccaaacagcaactcgtggatgtggacgactctcaatcctgcccagtacggaaaaatgatgtgggtgcaacgagacttcatgatctacaactattgtaccgattttaagagattccctcaaggcctccccaaggagtgtaaactttga

SEQ ID NO: 5 - DNA, 861 bp, Arabidopsis thaliana
atggcctgtttcgcaaccaaacagccgttgttgctctccttgttgctcgccatcggtttcttcgtggtggcagcctccgcaggaaacttctatgagtccttcgacatcacctggggcaacggaagggcgaacattttcgaaaacggtcagctcctcacttgtacgctcgacaaggtgtccggctccggtttccagtcgaagaaggagtacttgttcggcaagatcgacatgaagctcaagttggtggcaggtaactcggcaggtaccgtcacagcgtactatttgtcgtccaagggaactgcgtgggacgaaatcgacttcgagttcctcggcaaccgtacaggacacccctacactattcacaccaacgtcttcaccggaggcaagggtgatcgggagatgcagttcaggctctggttcgacccgacagcggatttccatacttacacggtgcattggaaccccgtcaacatcattttcctcgtcgacggaatccccatccgagtcttcaagaacaacgagaagaacggtgtggcgtatcccaaaaaccagccgatgcgcatctactcctcgttgtgggaagcggacgactgggccacagaaggcggacgcgtcaagatcgactggtcgaacgcaccgttcaaggcgtcgtaccggaacttcaacgaccagtcgtcctgttcgaggacttcgtcgtccaagtgggtcacctgtgaacccaactcgaactcgtggatgtggactactctcaaccctgcccagtacggcaagatgatgtgggtgcagagggacttcatgatctacaactattgtaccgatttcaaacgattccctcagggtctccccaaggaatgtaaactc

SEQ ID NO: 6 - protein, 287 aa, Arabidopsis thaliana
MACFATKQPLLLSLLLAIGFFVVAASAGNFYESFDITWGNGRANIFENGQLLTCTLDKVSGSGFQSKKEYLFGKIDMKLKLVAGNSAGTVTAYYLSSKGTAWDEIDFEFLGNRTGHPYTIHTNVFTGGKGDREMQFRLWFDPTADFHTYTVHWNPVNIIFLVDGIPIRVFKNNEKNGVAYPKNQPMRIYSSLWEADDWATEGGRVKIDWSNAPFKASYRNFNDQSSCSRTSSSKWVTCEPNSNSWMWTTLNPAQYGKMMWVQRDFMIYNYCTDFKRFPQGLPKECKL

SEQ ID NO: 7 - DNA, 50 bp, Artificial Sequence
  ARTIFICIAL DNA PRIMER
ttcctcaatcctctatatacacaactggccatgggctcgtccctctggac

SEQ ID NO: 8 - DNA, 48 bp, Artificial Sequence
  ARTIFICIAL DNA PRIMER
tgtcagtcacctctagttaattagatgtccctatcgcgtgtacactcg

SEQ ID NO: 9 - DNA, 29 bp, Artificial Sequence
  Artificial DNA Primer
taattaactagaggtgactgacacctggc

SEQ ID NO: 10 - DNA, 31 bp, Artificial Sequence
  Artificial DNA Primer
catggccagttgtgtatatagaggattgagg

SEQ ID NO: 11 - DNA, 36 bp, Artificial Sequence
  Artificial DNA Primer
acatgtctttgataagctagcgggccgcatcatgta

SEQ ID NO: 12 - DNA, 36 bp, Artificial Sequence
  Artificial DNA Primer
tacatgatgcggcccgctagcttatcaaagacatgt

SEQ ID NO: 13 - DNA, 41 bp, Artificial Sequence
  Artificial DNA Primer
ttaatcgccttgcagcacaccgcttcctcgctcactgactc

SEQ ID NO: 14 - DNA, 47 bp, Artificial Sequence
  Artificial DNA Primer
acaataaccctgataaatgcggaacaacactcaaccctatctcggtc

SEQ ID NO: 15 - DNA, 53 bp, Artificial Sequence
  Artificial DNA Primer
agatagggttgagtgttgttccgcatttatcagggttattgtctcatgagcgg

SEQ ID NO: 16 - DNA, 42 bp, Artificial Sequence
  Artificial DNA Primer
ttctacacgaaggaaagaggaggagagagttgaacctggacg

SEQ ID NO: 17 - DNA, 47 bp, Artificial Sequence
  Artificial DNA Primer
aggttcaactctctcctcctctttccttcgtgtagaagaccagacag

SEQ ID NO: 18 - DNA, 43 bp, Artificial Sequence
  Artificial DNA Primer
tcagtgagcgaggaagcggtgtgctgcaaggcgattaagttgg

SEQ ID NO: 19 - DNA, 54 bp, Artificial Sequence
  Artificial DNA Primer
ttcctcaatcctctatatacacaactggccatggcctgtttcgcaaccaaacag

SEQ ID NO: 20 - DNA, 47 bp, Artificial Sequence
  Artificial DNA Primer
agctcgctagagtcgacctagagtttacattccttggggagaccctg

SEQ ID NO: 21 - DNA, 27 bp, Artificial Sequence
  Artificial DNA Primer
taggtcgactctagcgagctcgagatc

SEQ ID NO: 22 - DNA, 40 bp, Artificial Sequence
  Artificial DNA Primer
catggccagttgtgtatatagaggattgaggaaggaagag

SEQ ID NO: 23 - DNA, 678 bp, Bacillus licheniformis
atggcttcaactgaagacgtaatcaaagagttcatgcgcttcaaagtgcgaatggaaggaagtgtaaacgggcatgagtttgaaattgaaggtgaaggtgaaggaaggccttatgaaggaacgcaaactgcaaaacttaaagtgacaaaaggaggaccgctgccgtttgcttgggacatcttaagtccgcagtttcagtatgggtcaaaagtttatgtaaagcatcctgctgacattcctgattacaaaaagttaagttttcctgaaggattcaagtgggagcgcgtaatgaactttgaagatggaggtgtcgtaactgtaacgcaagattcaagtctgcaagacggttgcttcatttacaaagtaaagttcattggcgtgaactttccaagtgatggtcctgtaatgcagaaaaagacaatgggttgggagccgtcaactgagaggctttatccgcgtgatggtgtcttgaaaggtgaaattcacaaagccttaaagttgaaagatggagggcattatcttgttgagttcaagagcatttacatggcgaaaaagcctgtgcagcttcctggctactactatgttgattcaaaacttgacataactagtcacaacgaagactacacaattgttgagcagtatgagcgaactgaaggaaggcatcatctttttctttaa

SEQ ID NO: 24 - protein, 225 aa, Bacillus licheniformis
MASTEDVIKEFMRFKVRMEGSVNGHEFEIEGEGEGRPYEGTQTAKLKVTKGGPLPFAWDILSPQFQYGSKVYVKHPADIPDYKKLSFPEGFKWERVMNFEDGGVVTVTQDSSLQDGCFIYKVKFIGVNFPSDGPVMQKKTMGWEPSTERLYPRDGVLKGEIHKALKLKDGGHYLVEFKSIYMAKKPVQLPGYYYVDSKLDITSHNEDYTIVEQYERTEGRHHLFL

SEQ ID NO: 25 - DNA, 24 bp, Artificial Sequence
  Artificial DNA Primer
acctgcctgtacacttgcgtcctc

SEQ ID NO: 26 - DNA, 23 bp, Artificial Sequence
  Artificial DNA Primer
ccatttcatccccgccttaccta

SEQ ID NO: 27 - DNA, 40 bp, Artificial Sequence
  Artificial DNA Primer
gactcactatagggaatattaagcttgctgctatgccggg

SEQ ID NO: 28 - DNA, 53 bp, Artificial Sequence
  Artificial DNA Primer
cgatttccaatgaggttaagagcctaggtgcatgaaggatggtcccgtttttg

SEQ ID NO: 29 - DNA, 48 bp, Artificial Sequence
  Artificial DNA Primer
gatccgaaccatttgatcatatgtctgacgtgtctgcggacaagttag

SEQ ID NO: 30 - DNA, 43 bp, Artificial Sequence
  Artificial DNA Primer
ggcggccgttactagtggatcctgcatgtttctccagcaattg

SEQ ID NO: 31 - DNA, 53 bp, Artificial Sequence
  Artificial DNA Primer
caaaaacgggaccatccttcatgcacctaggctcttaacctcattggaaatcg

SEQ ID NO: 32 - DNA, 48 bp, Artificial Sequence
  Artificial DNA Primer
ctaacttgtccgcagacacgtcagacatatgatcaaatggttcggatc

SEQ ID NO: 33 - DNA, 40 bp, Artificial Sequence; identical to SEQ ID NO: 27
  Artificial DNA Primer

SEQ ID NO: 34 - DNA, 40 bp, Artificial Sequence
  Artificial DNA Primer
cccggcatagcagcaagcttaatattccctatagtgagtc

SEQ ID NO: 35 - DNA, 52 bp, Artificial Sequence
  Artificial DNA Primer
gtcaaaaacgggaccatccttcatgcacctaggacctgcctgtacacttgcg

SEQ ID NO: 36 - DNA, 53 bp, Artificial Sequence
  Artificial DNA Primer
gatttccaatgaggttaagagcctaggccatttcatccccgccttacctatgc

SEQ ID NO: 37 - DNA, 53 bp, Artificial Sequence
  Artificial DNA Primer
gcataggtaaggcggggatgaaatggcctaggctcttaacctcattggaaatc

SEQ ID NO: 38 - DNA, 52 bp, Artificial Sequence
  Artificial DNA Primer
cgcaagtgtacaggcaggtcctaggtgcatgaaggatggtcccgtttttgac

SEQ ID NO: 39 - DNA, 40 bp, Artificial Sequence; identical to SEQ ID NO: 27
  Artificial DNA Primer

SEQ ID NO: 40 - DNA, 43 bp, Artificial Sequence; identical to SEQ ID NO: 30
  Artificial DNA Primer

(19) United States
(12) Patent Application Publication
(10) Pub. No.: US 2017/0027167 A1
(43) Pub. Date: Feb. 2, 2017

(54) FORMULATIONS COMPRISING POLYMERIC XYLOGLUCAN AS A CARRIER FOR AGRICULTURALLY BENEFICIAL AGENTS

(71) Applicant: NOVOZYMES BIOAG A/S, Bagsvaerd (DK)

(72) Inventors: Alex Berlin, Davis, CA (US); Jason Quinlan, Woodland, CA (US); Romil Benyamino, Sacramento, CA (US); Audrey Diano, Vacaville, CA (US)

(73) Assignee: NOVOZYMES BIOAG A/S, Bagsvaerd (DK)

(21) Appl. No.: 15/123,833

(22) PCT Filed: Mar. 5, 2015
(86) PCT No.: PCT/US2015/018849

§ 371 Date: Sep. 6, 2016

Related U.S. Application Data
(60) Provisional application No. 61/948,235, filed on Mar. 5, 2014.

(51) Int. Cl. A01N 25/24; C12N 9/10; A01N 25/10

(52) U.S. Cl. CPC A01N 25/24; A01N 25/10; C12N 9/1051; C12Y 204/01207

(57) ABSTRACT

The present invention relates to formulations comprising one or more agriculturally beneficial agents formulated with polymeric xyloglucan as a carrier and their use.

### REFERENCE TO A SEQUENCE LISTING

This application contains a Sequence Listing in computer readable form, which is incorporated herein by reference.

### BACKGROUND OF THE INVENTION

Field of the Invention

The present invention relates to formulations comprising xyloglucan as a carrier for agriculturally beneficial agents and methods of using such formulations.

Description of the Related Art

Xyloglucan endotransglycosylase (XET) is an enzyme that catalyzes endotransglycosylation of xyloglucan, a structural polysaccharide of plant cell walls. The enzyme is present in most plants, and in particular, land plants. XET has been extracted from dicotyledons and monocotyledons.

Xyloglucan is present in cotton, paper, or wood fibers (Hayashi et al., 1988, Carbohydrate Research 181: 273-277) making strong hydrogen bonds to cellulose (Carpita and Gibeaut, 1993, The Plant Journal 3: 1-30). Adding xyloglucan endotransglycosylase to various cellulosic materials containing xyloglucan alters the xyloglucan mediated interlinkages between cellulosic fibers improving their strength, and maintaining the cellulose-structure while permitting the cellulose fibers to move relative to one another under force.

There is a need in the art to improve crop yield by enhancing plant growth and preventing loss due to pest, disease or environmental factors. The current methods of enhancing plant growth, including fertilization, can be costly and environmentally damaging because the methods do not efficiently target the plants themselves. There is also a need in the art to develop carriers that provide nutrients or stimulating molecules to the plants themselves, or hold those molecules in locations that can be accessed by plant roots. There is also a need in the art to develop carriers for pesticides that hold the pesticides in the soil, or on the plants themselves, thereby rendering the plants pest-resistant, while minimizing the amount of pesticide that runs off or leaches into the soil.

The present invention provides formulations employing xyloglucan as a carrier for agriculturally beneficial agents and methods of using such formulations.

### SUMMARY OF THE INVENTION

The present invention relates to formulations comprising one or more (e.g., several) agriculturally beneficial agents formulated with a composition selected from the group consisting of (a) a composition comprising a xyloglucan endotransglycosylase, a polymeric xyloglucan, and a functionalized xyloglucan oligomer comprising a chemical group; (b) a composition comprising a xyloglucan endotransglycosylase, a polymeric xyloglucan functionalized with a chemical group, and a functionalized xyloglucan oligomer comprising a chemical group; (c) a composition comprising a xyloglucan endotransglycosylase, a polymeric xyloglucan functionalized with a chemical group, and a xyloglucan oligomer; (d) a composition comprising a xyloglucan endotransglycosylase, a polymeric xyloglucan, and a xyloglucan oligomer; (e) a composition comprising a xyloglucan endotransglycosylase and a polymeric xyloglucan functionalized with a chemical group; (f) a composition comprising a xyloglucan endotransglycosylase and a polymeric xyloglucan; (g) a composition comprising a xyloglucan endotransglycosylase and a functionalized xyloglucan oligomer comprising a chemical group; (h) a composition comprising a xyloglucan endotransglycosylase and a xyloglucan oligomer, and (i) a composition of (a), (b), (c), (d), (e), (f), (g), or (h) without a xyloglucan endotransglycosylase, wherein the formulation provides an agricultural benefit.

The present invention also relates to methods of formulating one or more (e.g., several) agriculturally beneficial agents, comprising reacting the one or more (e.g., several) agriculturally beneficial agents with a composition selected from the group consisting of (a) a composition comprising a xyloglucan endotransglycosylase, a polymeric xyloglucan, and a functionalized xyloglucan oligomer comprising a chemical group; (b) a composition comprising a xyloglucan endotransglycosylase, a polymeric xyloglucan functionalized with a chemical group, and a functionalized xyloglucan oligomer comprising a chemical group; (c) a composition comprising a xyloglucan endotransglycosylase, a polymeric xyloglucan functionalized with a chemical group, and a xyloglucan oligomer; (d) a composition comprising a xyloglucan endotransglycosylase, a polymeric xyloglucan, and a xyloglucan oligomer; (e) a composition comprising a xyloglucan endotransglycosylase and a polymeric xyloglucan functionalized with a chemical group; (f) a composition comprising a xyloglucan endotransglycosylase and a polymeric xyloglucan; (g) a composition comprising a xyloglucan endotransglycosylase and a functionalized xyloglucan oligomer comprising a chemical group; (h) a composition comprising a xyloglucan endotransglycosylase and a xyloglucan oligomer, and (i) a composition of (a), (b), (c), (d), (e), (f), (g), or (h) without a xyloglucan endotransglycosylase.

The present invention also relates to methods for enhancing plant growth, comprising applying a formulation of the present invention to a seed, a plant, a plant part, and/or a soil.

### BRIEF DESCRIPTION OF THE FIGURES

FIG. 1 shows a restriction map of pDLHD0012.

FIG. 2 shows a restriction map of pMMar27.

FIG. 3 shows a restriction map of pEvFz1.

FIG. 4 shows a restriction map of pDLHD0006.

FIG. 5 shows a restriction map of pDLHD0039.

FIG. 6 shows the decrease of fluorescence intensity of the supernatants of fluorescein isothiocyanate-labeled xyloglucan (FITC-XG) incubated with filter paper, indicating enhancement of cellulose-xyloglucan binding by Vigna angularis xyloglucan endotransglycosylase 16 (VaXET16).

FIG. 7 shows damaged raspberry leaf, undamaged raspberry leaf, and Whatman #1 filter paper, indicating the cutout rectangle used to assess FITC-XG binding.

FIG. 8 shows the fluorescence intensity of supernatants of FITC-XG to damaged and undamaged raspberry leaves.

FIG. 9 shows the binding capacity of cellulose for FITC-XG at various pH values in the presence and absence of VaXET16.

FIG. 10 shows the binding capacity of cellulose for FITC-XG at various temperatures in the presence and absence of VaXET16.

FIG. 11A shows the fluorescence intensity of the supernatants of undamaged leaf cuttings incubated with FITC-XG with or without VaXET16 as a function of incubation time and FIG. 11B shows the fluorescence intensity of the supernatants of damaged leaf cuttings incubated with FITC-XG with or without VaXET16 as a function of incubation time.

FIG. 12 shows laser scanning confocal microscope images (transmission on left and fluorescence emission on right) and comparing strawberry roots incubated with (panel A) sodium citrate pH 5.5, (panel B) FITC-XG in sodium citrate pH 5.5, and (panel C) FITC-XG with VaXET16 in sodium citrate pH 5.5 obtained using a 10× objective lens.

FIG. 13 shows laser scanning confocal microscope images comparing strawberry roots incubated with (panel A) sodium citrate pH 5.5, (panel B) FITC-XG in sodium citrate pH 5.5, and (panel C) FITC-XG with VaXET16 in sodium citrate pH 5.5 obtained using a 40× objective lens.

FIG. 14 shows laser scanning confocal microscope images (transmission on left and fluorescence emission on right) comparing tomato seed edges incubated with (panel A) sodium citrate pH 5.5, (panel B) FITC-XG in sodium citrate pH 5.5, and (panel C) FITC-XG with VaXET16 in sodium citrate pH 5.5 obtained using a 40× objective lens.

FIG. 15 shows laser scanning confocal microscope images (transmission on left and fluorescence emission on right) comparing tomato seed hairs incubated with (panel A) sodium citrate pH 5.5, (panel B) FITC-XG in sodium citrate pH 5.5, and (panel C) FITC-XG with VaXET16 in sodium citrate pH 5.5 obtained using a 40× objective lens.

FIG. 16 shows the relative optical densities of unbound TAEGRO® suspensions at 600 nm after incubation under ambient conditions for 24 hours in 20 mM sodium citrate pH 5.5 with or without 1 mg/ml tamarind seed xyloglucan, with or without 0.5 mg/ml microcrystalline cellulose, with or without 0.56 μM VaXET16, and with either a circular disc cutting of a raspberry leaf, or a circular disc cutting of filter paper.

FIG. 17 shows a photograph of a culture plate following a 12 hour incubation of variously incubated discs of BBL® Cefinase paper discs in Luria-Bertani (LB) medium. Discs were incubated with buffer and RFP-TAEGRO (top panel), buffer and RFP-TAEGRO with xyloglucan (middle panel), or buffer and RFP-TAEGRO® with xyloglucan and VaXET16 (bottom panel), then rinsed and LB medium was added. Darker suspensions indicated greater RFP production, thus more viable spores associated with the paper discs.

FIG. 18 shows the fluorescence spectra of LB medium inoculated with variously incubated BBL® Cefinase paper discs incubated in citrate buffer (solid gray lines); incubated with RFP-TAEGRO (dashed gray lines); incubated with RFP-TAEGRO and xyloglucan (XG) (dashed black lines); and incubated with RFP-TAEGRO, XG and VaXET16 (solid black lines).

### DEFINITIONS

As used herein, the singular forms “a”, “an”, and “the” are intended to include the plural forms as well, unless the context clearly indicates otherwise.

Acaricide: The term “acaricide” means any agent or combination of agents capable of killing one or more acarids and/or inhibiting the growth of one or more acarids.

Agriculturally beneficial agent: The term “agriculturally beneficial agent” means any agent or combination of agents capable of causing or providing a beneficial and/or useful effect in agriculture.

Anthocyanidin: The term “anthocyanidin” means anthocyanidins, cyanidins, delphinidins, malvidins, pelargonidins, peonidins, and petunidins.

Biostimulant: The term “biostimulant” means any agent or combination of agents capable of enhancing metabolic or physiological processes within plants and soils.

Carrier: The term “carrier” means an agronomically acceptable carrier comprising functionalized or unfunctionalized polymeric xyloglucan and/or xyloglucan oligomer for delivering one or more (e.g., several) agriculturally beneficial agents to a seed, a plant, a plant part (e.g., plant foliage), or a soil and, preferably, which can be applied (to the seed, plant, plant part (e.g., foliage), or soil) without having an adverse effect on plant growth, soil structure, soil drainage, or the like.

Effective amount, effective concentration, or effective dosage: The terms “effective amount”, “effective concentration”, and “effective dosage” mean the amount, concentration, or dosage of one or more agriculturally beneficial agents sufficient to cause a desired agricultural benefit. The actual effective dosage in absolute value depends on factors including, but not limited to, the size (e.g., the area, the total acreage, etc.) of land for application with the one or more agriculturally beneficial agents, synergistic or antagonistic interactions between the agriculturally beneficial agents, which may increase or reduce the growth enhancing effects of the one or more agriculturally beneficial agents, and the stability of the one or more agriculturally beneficial agents in compositions and/or as plant or plant part treatments. The “effective amount”, “effective concentration”, or “effective dosage” of the one or more agriculturally beneficial agents may be determined, e.g., by routine dose response.

Enhanced plant growth or enhancing plant growth: The terms “enhanced plant growth” and “enhancing plant growth” mean increased plant yield (e.g., increased biomass, increased fruit number, or a combination thereof that may be measured by bushels per acre), increased root number, increased root mass, increased root volume, increased leaf area, increased plant stand, increased plant vigor, faster seedling emergence (i.e., enhanced emergence), faster germination, (i.e., enhanced germination), increased bolls, or combinations thereof.

Flavanol: The term “flavanol” means flavan-3-ols (e.g., catechin, gallocatechin, catechin 3-gallate, gallocatechin 3-gallate, epicatechins, epigallocatechin, epicatechin 3-gallate, epigallocatechin 3-gallate, etc.), flavan-4-ols, flavan-3,4-diols (e.g., leucoanthocyanidin), and proanthocyanidins (e.g., includes dimers, trimer, oligomers, or polymers of flavanols).

Flavones: The term “flavones” means without limitation flavones (e.g., luteolin, apigenin, tangeritin, etc.), flavonols (e.g., quercetin, quercitrin, rutin, kaempferol, kaempferitrin, astragalin, sophoraflavonoloside, myricetin, fisetin, isorhamnetin, pachypodol, rhamnazin, etc.), flavanones (e.g., hesperetin, hesperidin, naringenin, eriodictyol, homoeriodictyol, etc.), and flavanonols (e.g., dihydroquercetin, dihydrokaempferol, etc.). Flavonoid: The term “flavonoid” means flavanols, flavones, anthocyanidins, isoflavonoids, neoflavonoids and all isomer, solvate, hydrate, polymorphic, crystalline, non-crystalline, and salt variations thereof.

Foliage: The term “foliage” means all parts and organs of plants above ground. Non-limiting examples include leaves, needles, stalks, stems, flowers, fruit bodies, fruits, etc. Foliar application or foliarly applied: The terms “foliar application”, “foliarly applied”, and variations thereof, mean application of an agriculturally beneficial agent to foliage or above ground portions of a plant, (e.g., the leaves of the plant). Application may be effected by any means known in the art (e.g., spraying the active agent).

Foliar-compatible carrier: The term “foliar-compatible carrier” means a xyloglucan carrier that can be added to a seed, a plant, a plant part, or a soil without causing or having an adverse effect on the plant, plant part, plant growth, plant health, or the like.

Functionalized xyloglucan oligomer: The term “functionalized xyloglucan oligomer” means a short chain xyloglucan oligosaccharide, including single or multiple repeating units of xyloglucan, which has been modified by incorporating a chemical group. The xyloglucan oligomer is preferably 1 to 3 kDa in molecular weight, corresponding to 1 to 3 repeating xyloglucan units. The chemical group may be a compound of interest or a reactive group such as an aldehyde group, an amino group, an aromatic group, a carboxyl group, a halogen group, a hydroxyl group, a ketone group, a nitrile group, a nitro group, a sulfhydryl group, or a sulfonate group. The incorporated reactive groups can be derivatized with a compound of interest to provide a direct agricultural benefit or to coordinate metal cations and/or to bind other chemical entities that interact (e.g., covalently, hydrophobically, electrostatically, etc.) with the reactive groups. The derivatization can be performed directly on a functionalized xyloglucan oligomer comprising a reactive group or after the functionalized xyloglucan oligomer comprising a reactive group is incorporated into polymeric xyloglucan. Alternatively, the xyloglucan oligomer can be functionalized by incorporating directly a compound by using a reactive group contained in the compound, e.g., an aldehyde group, an amino group, an aromatic group, a carboxyl group, a halogen group, a hydroxyl group, a ketone group, a nitrile group, a nitro group, a sulfhydryl group, or a sulfonate group. The terms “functionalized xyloglucan oligomer” and “functionalized xyloglucan oligomer comprising a chemical group” are used interchangedly herein.

Fungicide: The term “fungicide” means any agent or combination of agents capable of killing fungi and/or inhibiting fungal growth.

Herbicide: The term “herbicide” means any agent or combination of agents capable of killing weeds and/or inhibiting the growth of weeds (the inhibition being reversible under certain conditions).

Inoculum: The term “inoculum” means any form of microbial cells, or spores, which is capable of propagating on or in the soil when the conditions of temperature, moisture, etc., are favorable for microbial growth.

Insecticide: The term “insecticide” means any agent or combination of agents capable of killing one or more insects and/or inhibiting the growth of one or more insects.

Isoflavonoid: The term “isoflavonoid” means phytoestrogens, isoflavones (e.g., genistein, daidzein, glycitein, etc.), and isoflavanes (e.g., equol, lonchocarpane, laxiflorane, etc.).

Isomer: The term “isomer” includes all stereoisomers of the compounds and/or molecules referred to herein (e.g., flavonoids, lipo-chitooligosaccharides (LCOs), chitooligosaccharides (COs), chitinous compounds, jasmonic acid or derivatives thereof, linoleic acid or derivatives thereof, linolenic acid or derivatives thereof, kerrikins, etc.), including enantiomers, diastereomers, positional isomers, as well as all conformers, rotamers, and tautomers. The compounds and/or molecules disclosed herein include all enantiomers in either substantially pure levorotatory or dextrorotatory form, or in a racemic mixture, or in any ratio of enantiomers. Where an embodiment is a (D)-enantiomer, that embodiment also includes the (L)-enantiomer; where an embodiment is a (L)-enantiomer, that embodiment also includes the (D)-enantiomer. Where an embodiment is a (+)-enantiomer, that embodiment also includes the (−)-enantiomer; where an embodiment is a (−)-enantiomer, that embodiment also includes the (+)-enantiomer. Where an embodiment is a (S)-enantiomer, that embodiment also includes the (R)-enantiomer; where an embodiment is a (R)-enantiomer, that embodiment also includes the (S)-enantiomer. Embodiments are intended to include any diastereomers of the compounds and/or molecules referred to herein in diastereomerically pure form and in the form of mixtures in all ratios. Unless stereochemistry is explicitly indicated in a chemical structure or chemical name, the chemical structure or chemical name is intended to embrace all possible stereoisomers, conformers, rotamers, and tautomers of compounds and/or molecules depicted.

Microbially stabilizing compound: The term “microbially stabilizing compound” means any compound capable of maintaining and/or increasing the viability, survivability, and/or colony forming units (CFU) of one or more microbes. As used herein a “microbially stabilizing compound” is further intended to mean any compound capable of preventing and/or decreasing the amount of death and/or rate of death of one or more microbes. Nematode-antagonistic agent: The term “nematode-antagonistic agent” means any agent or combination of agents that inhibit nematode activity, growth or reproduction, or reduces nematode-related disease in plants, or which releases or contains substances toxic or inhibitory to nematodes.

Neoflavonoid: The term “neoflavonoid” means neoflavones (e.g., calophyllolide), neoflavenes (e.g., dalbergichromene), coutareagenins, dalbergins, and nivetins.

Nitrogen-fixing organism: The term “nitrogen-fixing organism” means any organism capable of converting atmospheric nitrogen (N₂) into ammonia (NH₃).

Nutrient: The term “nutrient” means compounds (e.g., vitamins, macrominerals, trace minerals, organic acids, etc.) that are needed for plant growth, plant health, and/or plant development.

Plant and plant part: The terms “plant” and “plant part” mean all plants and plant populations such as desired and undesired wild plants or crop plants (including naturally occurring crop plants). Crop plants can be obtained by conventional plant breeding and optimization methods or by biotechnological and genetic engineering methods or by combinations of these methods, including transgenic plants and including plant cultivars protectable or not protectable by plant breeders' rights. Plant parts are to be understood as meaning all parts and organs of plants above and below the ground, such as flowers, fruit bodies, fruits, leaves, needles, seeds, shoots, stalks, stems, roots, tubers and rhizomes. The plant parts also include harvested material and vegetative and generative propagation material (e.g., cuttings, tubers, rhizomes, off-shoots and seeds, etc.).

Phosphate solubilizing organism: The term “phosphate solubilizing organism” means any organism capable of converting insoluble phosphate into a soluble phosphate form.

Polymeric xyloglucan: The term “polymeric xyloglucan” means short, intermediate or long chain xyloglucan oligosaccharide or polysaccharide encompassing more than one repeating unit of xyloglucan, e.g., multiple repeating units of xyloglucan. Most optimally, polymeric xyloglucan encompasses xyloglucan of 50-200 kDa number average molecular weight, corresponding to 50-200 repeating units. A repeating motif of xyloglucan is composed of a backbone of four beta-(1-4)-D-glucopyranose residues, three of which have a single alpha-D-xylopyranose residue attached at O-6. Some of the xylose residues are beta-D-galactopyranosylated at O-2, and some of the galactose residues are alpha-L-fucopyranosylated at O-2. The term “xyloglucan” herein is understood to mean polymeric xyloglucan.

Polymeric xyloglucan functionalized with a chemical group: The term “polymeric xyloglucan functionalized with a chemical group” means a polymeric xyloglucan that has been modified by incorporating a chemical group. The polymeric xyloglucan is short, intermediate or long chain xyloglucan oligosaccharide or polysaccharide encompassing more than one repeating unit of xyloglucan, e.g., multiple repeating units of xyloglucan. The polymeric xyloglucan encompasses xyloglucan of 50-200 kDa number average molecular weight, corresponding to 50-200 repeating units. A repeating motif of xyloglucan is composed of a backbone of four beta-(1-4)-D-glucopyranose residues, three of which have a single alpha-D-xylopyranose residue attached at O-6. The chemical group may be a compound of interest or a reactive group such as an aldehyde group, an amino group, an aromatic group, a carboxyl group, a halogen group, a hydroxyl group, a ketone group, a nitrile group, a nitro group, a sulfhydryl group, or a sulfonate group. The chemical group can be incorporated into a polymeric xylogucan by reacting the polymeric xyloglucan with a functionalized xyloglucan oligomer in the presence of xyloglucan endotransglycosylase. The incorporated reactive groups can then be derivatized with a compound of interest. The derivatization can be performed directly on a functionalized polymeric xyloglucan comprising a reactive group or after a functionalized xyloglucan oligomer comprising a reactive group is incorporated into a polymeric xyloglucan. Alternatively, the polymeric xyloglucan can be functionalized by incorporating directly a compound by using a reactive group contained in the compound, e.g., an aldehyde group, an amino group, an aromatic group, a carboxyl group, a halogen group, a hydroxyl group, a ketone group, a nitrile group, a nitro group, a sulfhydryl group, or a sulfonate group.

Spore: The term “spore” means a microorganism in its dormant, protected state.

Xyloglucan endotransglycosylase: The term “xyloglucan endotransglycosylase” means a xyloglucan:xyloglucan xyloglucanotransferase (EC 2.4.1.207) that catalyzes cleavage of a β-(1→4) bond in the backbone of a xyloglucan and transfers the xyloglucanyl segment on to O-4 of the non-reducing terminal glucose residue of an acceptor, which can be a xyloglucan or an oligosaccharide of xyloglucan. Xyloglucan endotransglycosylases are also known as xyloglucan endotransglycosylase/hydrolases or endo-xyloglucan transferases. Some xylan endotransglycosylases can possess different activities including xyloglucan and mannan endotransglycosylase activities. For example, xylan endotransglycosylase from ripe papaya fruit can use heteroxylans, such as wheat arabinoxylan, birchwood glucuronoxylan, and others as donor molecules. These xylans can potentially play a similar role as xyloglucan while being much cheaper in cost since they can be extracted, for example, from pulp mill spent liquors and/or future biomass biorefineries.

Xyloglucan endotransglycosylase activity can be assayed by those skilled in the art using any of the following methods. The reduction in the average molecular weight of a xyloglucan polymer when incubated with a molar excess of xyloglucan oligomer in the presence of xyloglucan endotransglycosylase can be determined via liquid chromatography (Sulova et al., 2003, Plant Physiol. Biochem. 41: 431-437) or via ethanol precipitation (Yaanaka et al., 2000, Food Hydrocolloids 14: 125-128) followed by gravimetric or cellulose-binding analysis (Fry et al., 1992, Biochem. J. 282: 821-828), or can be assessed colorimetrically by association with iodine under alkaline conditions (Sulova et al., 1995, Analytical Biochemistry 229: 80-85). Incorporation of a functionalized xyloglucan oligomer into a xyloglucan polymer by incubation of the functionalized oligomer with xyloglucan in the presence of xyloglucan endotransglycosylase can be assessed, e.g., by incubating a radiolabeled xyloglucan oligomer with xyloglucan and xyloglucan endotransglycosylase, followed by filter paper-binding and measurement of filter paper radioactivity, or incorporation of a fluorescently or optically functionalized xyloglucan oligomer can be assessed similarly, monitoring fluorescence or colorimetrically analyzing the filter paper.

Xyloglucan oligomer: The term “xyloglucan oligomer” means a short chain xyloglucan oligosaccharide, including single or multiple repeating units of xyloglucan. Most optimally, the xyloglucan oligomer will be 1 to 3 kDa in molecular weight, corresponding to 1 to 3 repeating xyloglucan units.

### DETAILED DESCRIPTION OF THE INVENTION

The present invention relates to formulations comprising one or more (e.g., several) agriculturally beneficial agents formulated with a composition selected from the group consisting of (a) a composition comprising a xyloglucan endotransglycosylase, a polymeric xyloglucan, and a functionalized xyloglucan oligomer comprising a chemical group; (b) a composition comprising a xyloglucan endotransglycosylase, a polymeric xyloglucan functionalized with a chemical group, and a functionalized xyloglucan oligomer comprising a chemical group; (c) a composition comprising a xyloglucan endotransglycosylase, a polymeric xyloglucan functionalized with a chemical group, and a xyloglucan oligomer; (d) a composition comprising a xyloglucan endotransglycosylase, a polymeric xyloglucan, and a xyloglucan oligomer; (e) a composition comprising a xyloglucan endotransglycosylase and a polymeric xyloglucan functionalized with a chemical group; (f) a composition comprising a xyloglucan endotransglycosylase and a polymeric xyloglucan; (g) a composition comprising a xyloglucan endotransglycosylase and a functionalized xyloglucan oligomer comprising a chemical group; (h) a composition comprising a xyloglucan endotransglycosylase and a xyloglucan oligomer, and (i) a composition of (a), (b), (c), (d), (e), (f), (g), or (h) without a xyloglucan endotransglycosylase, wherein the formulation provides an agricultural benefit.

The present invention also relates to methods of formulating one or more (e.g., several) agriculturally beneficial agents, comprising reacting the one or more (e.g., several) agriculturally beneficial agents with a composition selected from the group consisting of (a) a composition comprising a xyloglucan endotransglycosylase, a polymeric xyloglucan, and a functionalized xyloglucan oligomer comprising a chemical group; (b) a composition comprising a xyloglucan endotransglycosylase, a polymeric xyloglucan functionalized with a chemical group, and a functionalized xyloglucan oligomer comprising a chemical group; (c) a composition comprising a xyloglucan endotransglycosylase, a polymeric xyloglucan functionalized with a chemical group, and a xyloglucan oligomer; (d) a composition comprising a xyloglucan endotransglycosylase, a polymeric xyloglucan, and a xyloglucan oligomer; (e) a composition comprising a xyloglucan endotransglycosylase and a polymeric xyloglucan functionalized with a chemical group; (f) a composition comprising a xyloglucan endotransglycosylase and a polymeric xyloglucan; (g) a composition comprising a xyloglucan endotransglycosylase and a functionalized xyloglucan oligomer comprising a chemical group; (h) a composition comprising a xyloglucan endotransglycosylase and a xyloglucan oligomer, and (i) a composition of (a), (b), (c), (d), (e), (f), (g), or (h) without a xyloglucan endotransglycosylase.

In one embodiment, the composition comprises a xyloglucan endotransglycosylase, a polymeric xyloglucan, and a functionalized xyloglucan oligomer comprising a chemical group. In another embodiment, the composition comprises a xyloglucan endotransglycosylase, a polymeric xyloglucan functionalized with a chemical group, and a functionalized xyloglucan oligomer comprising a chemical group. In another embodiment, the composition comprises a xyloglucan endotransglycosylase, a polymeric xyloglucan functionalized with a chemical group, and a xyloglucan oligomer. In another embodiment, the composition comprises a xyloglucan endotransglycosylase, a polymeric xyloglucan, and a xyloglucan oligomer. In another embodiment, the composition comprises a xyloglucan endotransglycosylase and a polymeric xyloglucan functionalized with a chemical group. In another embodiment, the composition comprises a xyloglucan endotransglycosylase and a polymeric xyloglucan. In another embodiment, the composition comprises a xyloglucan endotransglycosylase and a functionalized xyloglucan oligomer comprising a chemical group. In another embodiment, the composition comprises a xyloglucan endotransglycosylase and a xyloglucan oligomer.

In another embodiment, the composition comprises a polymeric xyloglucan and a functionalized xyloglucan oligomer comprising a chemical group. In another embodiment, the composition comprises a polymeric xyloglucan functionalized with a chemical group and a functionalized xyloglucan oligomer comprising a chemical group. In another embodiment, the composition comprises a polymeric xyloglucan functionalized with a chemical group and a xyloglucan oligomer. In another embodiment, the composition comprises a polymeric xyloglucan and a xyloglucan oligomer. In another embodiment, the composition comprises a polymeric xyloglucan functionalized with a chemical group. In another embodiment, the composition comprises a polymeric xyloglucan. In another embodiment, the composition comprises a functionalized xyloglucan oligomer comprising a chemical group. In another embodiment, the composition comprises a xyloglucan oligomer.

In one aspect, the functionalization can provide any functionally useful chemical moiety.

In one aspect, the agriculturally beneficial agent is covalently bound to the polymeric xyloglucan as a carrier. In another aspect, the agriculturally beneficial agent is electrostatically bound to the polymeric xyloglucan as a carrier. In another aspect, the agriculturally beneficial agent is hydrophobically bound to the polymeric xyloglucan as a carrier. In another aspect, the agriculturally beneficial agent is embedded into the polymeric xyloglucan as a carrier. In another aspect, the agriculturally beneficial agent is coated with polymeric xyloglucan as a carrier. In another aspect, the agriculturally beneficial agent is encapsulated within polymeric xyloglucan as a carrier.

Inclusion of xyloglucan endotransglycosylase in the formulation can alter the xyloglucan mediated interlinkages between the polymeric xyloglucan and natural xyloglucan in a plant, leading to covalent association of the xyloglucan or functionalized xyloglucan in the formulation with the xyloglucan of the plant tissue. Inclusion of xyloglucan endotransglycosylase in the formulation can also alter the xyloglucan, such that more xyloglucan or functionalized xyloglucan can be made to associate with exposed plant cellulose on the plant surface, unexposed cellulose within plant tissues or in the soil, or that xyloglucan or functionalized xyloglucan has greater affinity for sources of cellulose. It is particularly unanticipated that xyloglucan can be used to facilitate binding to plant leaf surfaces, despite the presence of a waxy cuticle layer that would be expected to prevent this association.

The xyloglucan endotransglycosylase is preferably present at about 0.1 nM to about 1 mM, e.g., about 10 nM to about 100 μM or about 0.5 μM to about 5 μM, in the formulation.

The polymeric xyloglucan or polymeric xyloglucan functionalized with a chemical group is preferably present at about 1 mg to about 1 g per g of the formulation, e.g., about 10 mg to about 950 mg per g or about 100 mg to about 900 mg per g of the formulation. Alternatively, the polymeric xyloglucan or polymeric xyloglucan functionalized with a chemical group can be present at a lower amount of about 0.1 μg to about 1 mg per g of the formulation, e.g., about 0.5 μg to about 1 mg, about 1 μg to about 1 mg, about 10 μg to about 1 mg, about 50 μg to about 1 mg, or about 100 μg to about 1 mg per g of the formulation. In one embodiment, the polymeric xyloglucan or polymeric xyloglucan functionalized with a chemical group is present at about 0.1 μg to about 1 g per g of the formulation

When the xyloglucan oligomer or the functionalized xyloglucan oligomer is present without polymeric xyloglucan or polymeric xyloglucan functionalized with a chemical group, the xyloglucan oligomer or the functionalized xyloglucan oligomer is preferably present at about 1 mg to about 1 g per g of the formulation, e.g., about 10 mg to about 950 mg or about 100 mg to about 900 mg per g of the formulation. Alternatively, the xyloglucan oligomer or the functionalized xyloglucan oligomer can be present at a lower amount of about 0.1 μg to about 1 mg per g of the formulation, e.g., about 0.5 μg to about 1 mg, about 1 μg to about 1 mg, about 10 μg to about 1 mg, about 50 μg to about 1 mg, or about 100 μg to about 1 mg per g of the formulation.

When present with polymeric xyloglucan, the xyloglucan oligomer or the functionalized xyloglucan oligomer is preferably present with the polymeric xyloglucan at about 50:1 to about 0.5:1 molar ratio of xyloglucan oligomer or functionalized xyloglucan oligomer to polymeric xyloglucan or polymeric xyloglucan functionalized with a chemical group, e.g., about 10:1 to about 1:1 or about 5:1 to about 1:1 molar ratio of xyloglucan oligomer or functionalized xyloglucan oligomer to polymeric xyloglucan or polymeric xyloglucan functionalized with a chemical group.

The present invention also relates to methods for enhancing plant growth, comprising applying a formulation of the present invention to a seed, a plant, a plant part, and/or a soil.

The polymeric xyloglucan or polymeric xyloglucan functionalized with a chemical group is preferably present at about 1 ng to about 1 g per g of a seed, a plant, a plant part, and/or a soil, e.g., about 10 μg to about 100 mg or about 1 mg to about 50 mg per g of a seed, a plant, a plant part, and/or a soil.

When the xyloglucan oligomer or the functionalized xyloglucan oligomer is present without polymeric xyloglucan or polymeric xyloglucan functionalized with a chemical group, the xyloglucan oligomer or the functionalized xyloglucan oligomer is preferably present at about 1 ng to about 1 g per g of a seed, a plant, a plant part, and/or a soil, e.g., about 10 mg to about 100 mg or about 20 mg to about 50 mg per g of a seed, a plant, a plant part, and/or a soil.

When present with polymeric xyloglucan, the xyloglucan oligomer or the functionalized xyloglucan oligomer is preferably present with the polymeric xyloglucan at about 50:1 to about 0.5:1 molar ratio of xyloglucan oligomer or functionalized xyloglucan oligomer to polymeric xyloglucan, e.g., about 10:1 to about 1:1 or about 5:1 to about 1:1 molar ratio of xyloglucan oligomer or functionalized xyloglucan oligomer to polymeric xyloglucan.

The xyloglucan endotransglycosylase is preferably present at about 0.1 nM to about 1 mM, e.g., about 10 nM to about 100 μM or about 0.5 μM to about 5 μM.

The concentration of polymeric xyloglucan, polymeric xyloglucan functionalized with a chemical group, xyloglucan oligomer, or functionalized xyloglucan oligomer comprising a chemical group incorporated into the material is about 0.01 g to about 500 mg per g of a seed, a plant, a plant part, and/or a soil, e.g., about 0.1 g to about 50 mg or about 1 to about 5 mg per g of a seed, a plant, a plant part, and/or a soil.

### Agricultural Benefits

Application of a formulation of the present invention to a seed, a plant, a plant part, and/or a soil can result in an agricultural benefit. The agricultural benefit can be one or more properties that enhance plant growth.

In one aspect, the agricultural benefit may be improved activity of an agriculturally beneficial agent. This improved activity may be due to better targeting to plants, better retention in soil, etc., leading to higher local and accessible concentrations of the beneficial agent.

In another aspect, the agricultural benefit is improved adhesion to plants or plant parts. The plant parts can be roots, shoots, stems, leaves, flowers, fruit, cotyledons, trunks, branches or other plant parts. Application of the agriculturally beneficial agent is enhanced via the natural affinity of xyloglucan for cellulose, and particularly by the enhancement of the binding capacity of xyloglucan to cellulose via xyloglucan endotransglycosylase.

In another aspect, the agricultural benefit is improved adhesion to soil components. The soil components can be organic (humic and non-humic) matter derived from plant, microbial or animal sources and may be more or less transformed, or the soil components can be mineral matter. The improved adhesion to soil components prevents run-off of the agriculturally beneficial agent(s).

In another aspect, the agricultural benefit can be improved uptake, accessibility, or incorporation by plants. Xyloglucan has a natural affinity for cellulose. Agriculturally beneficial agents linked to or linked by xyloglucan or functionalized xyloglucan can now be associated with cellulose or other polymers present in the soil or on plant parts themselves, thereby limiting the agriculturally beneficial agent's water-dependent or vapor-phase mobility and maintaining it near the root structure of the plant.

In another aspect, the agricultural benefit is increased resistance to sunlight or UV.

In another aspect, the agricultural benefit is prevention of, delay in, or reduction of infestation by agricultural pests. The agricultural pests may be an insect, fungus, animal, bacterium, virus, nematode, mite, or any other agricultural pest.

In another aspect, the agricultural benefit is resistance to physical damage.

In another aspect, the agricultural benefit is improved resistance to run-off (i.e., improved partitioning between soil and water). Binding of the agriculturally beneficial agent to insoluble cellulose, other biopolymers or minerals through xyloglucan or functionalized xyloglucan maintains adsorption of the agriculturally beneficial agent to the solid phase of the soil.

In another aspect, the agricultural benefit is reduced evaporation or volatilization.

In another aspect, the agricultural benefit is enhanced water or solvent solubility. Many agriculturally beneficial agents, particularly pesticides, are sparingly soluble in aqueous solvent. Linking of an insoluble agriculturally beneficial agent to a large, water-soluble xyloglucan or xyloglucan with functionalization tailored for solubility in the desired solvent can impart desired or enhanced solubility, allowing easier delivery to the field. Thus, using xyloglucan or functionalized xyloglucan as a carrier has the potential dual benefit of both increasing solubility in water, while simultaneously reducing water-dependent run-off by associating with cellulose in the soil.

In another aspect, the agricultural benefit is specific release of the agriculturally beneficial agent caused by direct or indirect fungal or microbial activity. Cellulose degrading fungi and bacteria are common plant pathogens, living independently or within the gut symbiome of plant-eating animal and insect pests, and secrete a suite of cellulases, hemicellulases, accessory enzymes, or combinations thereof, designed to damage or break down the plant. Many of the secreted cellulases and hemicellulases can degrade either cellulose or xyloglucan, thereby releasing a xyloglucan-linked agriculturally beneficial agent.

In another aspect, the agricultural benefit is improved plant tissue-specific targeting, or improved targeting of the agriculturally beneficial material to tissues within the plant. In many cases, pesticides must be taken up by plants and incorporated into their tissues, prior to attack by the plant. To bypass this route, addition of xyloglucan endotransglycosylase and xyloglucan linked or associated agriculturally beneficial agent can specifically target the agriculturally beneficial agent-xyloglucan within the plant tissues (e.g., leaves). Xyloglucan-linked or associated agriculturally beneficial agent can also be used as a dip or coating for seeds or fruit.

In another aspect the agricultural benefit is improved time of release. The time of release may be a delayed release, a controlled release, a release dependent on a plant or pest-specific activity, or a release dependent on the addition of another agent, composition or material. Germinating seeds produce cellulases, hemicellulases, and in some cases xyloglucanases to break down storage polysaccharides and the seed coat, facilitating release of nutrients or other agriculturally beneficial agents at the time and site of seed germination.

### Polymeric Xyloglucan

In the present invention, polymeric xyloglucan is suitable as a carrier. The polymeric xyloglucan can be any xyloglucan. In one aspect, the polymeric xyloglucan is obtained from natural sources. In another aspect, the polymeric xyloglucan is synthesized from component carbohydrates, UDP- or GDP-carbohydrates, or halogenated carbohydrates by any means used by those skilled in the art. In another aspect, the natural source of polymeric xyloglucan is tamarind seed or tamarind kernel powder, nasturtium, or plants of the genus Tropaeolum, particularly Tropaeolum majus. The natural source of polymeric xyloglucan may be seeds of various dicotyledonous plants such as Hymenaea courbaril, Leguminosae-Caesalpinioideae including the genera Cynometreae, Amherstieae, and Sclerolobieae. The natural source of polymeric xyloglucan may also be the seeds of plants of the families Primulales, Annonaceae, Limnanthaceae, Melianthaceae, Pedaliaceae, and Tropaeolaceae or subfamily Thunbergioideae. The natural source of polymeric xyloglucan may also be the seeds of plants of the families Balsaminaceae, Acanthaceae, Linaceae, Ranunculaceae, Sapindaceae, and Sapotaceae or non-endospermic members of family Leguminosae subfamily Faboideae. In another aspect, the natural source of polymeric xyloglucan is the primary cell walls of dicotyledonous plants. In another aspect, the natural source of polymeric xyloglucan may be the primary cell walls of nongraminaceous, monocotyledonous plants.

The natural source polymeric xyloglucan may be extracted by extensive boiling or hot water extraction, or by other methods known to those skilled in the art. In one aspect, the polymeric xyloglucan may be subsequently purified, for example, by precipitation in 80% ethanol. In another aspect, the polymeric xyloglucan is a crude or enriched preparation, for example, tamarind kernel powder. In another aspect, the synthetic xyloglucan may be generated by automated carbohydrate synthesis (Seeberger, Chem. Commun, 2003, 1115-1121), or by means of enzymatic polymerization, for example, using a glycosynthase (Spaduit et al., 2011, J. Am. Chem. Soc. 133: 10892-10900).

In one aspect, the average molecular weight of the polymeric xyloglucan ranges from about 2 kDa to about 500 kDa, e.g., about 2 kDa to about 400 kDa, about 3 kDa to about 300 kDa, about 3 kDa to about 200 kDa, about 5 kDa to about 100 kDa, about 5 kDa to about 75 kDa, about 7.5 kDa to about 50 kDa, or about 10 kDa to about 30 kDa. In another aspect, the number of repeating units is about 2 to about 500, e.g., about 2 to about 400, about 3 to about 300, about 3 to about 200, about 5 to about 100, about 7.5 to about 50, or about 10 to about 30. In another aspect, the repeating unit is any combination of G, X, L, F, S, T and J subunits, according to the nomenclature of Fry et al. (Physiologia Plantarum, 89: 1-3, 1993). In another aspect, the repeating unit is either fucosylated or non-fucosylated XXXG-type polymeric xyloglucan common to dicotyledons and nongraminaceous monocots. In another aspect, the polymeric xyloglucan is O-acetylated. In another aspect the polymeric xyloglucan is not O-acetylated. In another aspect, side chains of the polymeric xyloglucan may contain terminal fucosyl residues. In another aspect, side chains of the polymeric xyloglucan may contain terminal arabinosyl residues. In another aspect, side chains of the polymeric xyloglucan may contain terminal xylosyl residues.

For purposes of the present invention, references to the term xyloglucan herein refer to polymeric xyloglucan.

### Xyloglucan Oligomer

In the methods of the present invention, the xyloglucan oligomer can be any xyloglucan oligomer. The xyloglucan oligomer may be obtained by degradation or hydrolysis of polymeric xyloglucan from any source. The xyloglucan oligomer may be obtained by enzymatic degradation of polymeric xyloglucan, e.g., by quantitative or partial digestion with a xyloglucanase or endoglucanase (endo-β-1-4-glucanase). The xyloglucan oligomer may be synthesized from component carbohydrates, UDP- or GDP-carbohydrates, or halogenated carbohydrates by any of the manners commonly used by those skilled in the art.

In one aspect, the average molecular weight of the xyloglucan oligomer ranges from 0.5 kDa to about 500 kDa, e.g., about 1 kDa to about 20 kDa, about 1 kDa to about 10 kDa, or about 1 kDa to about 3 kDa. In another aspect, the number of repeating units is about 1 to about 500, e.g., about 1 to about 20, about 1 to about 10, or about 1 to about 3. In the methods of the present invention, the xyloglucan oligomer is optimally as short as possible (i.e., 1 repeating unit, or about 1 kDa in molecular weight) to maximize the solubility and solution molarity per gram of dissolved xyloglucan oligomer, while maintaining substrate specificity for xyloglucan endotransglycosylase activity. In another aspect, the xyloglucan oligomer comprises any combination of G (β-D glucopyranosyl-), X (α-D-xylopyranosyl-(1→6)-β-D-glucopyranosyl-), L (β-D-galactopyranosyl-(1→2)-α-D-xylopyranosyl-(1→6)-β-D-glucopyranosyl-), F (α-L-fuco-pyranosyl-(1→2)-β-D-galactopyranosyl-(1→2)-α-D-xylopyranosyl-(1→6)-β-D-glucopyranosyl-), S (α-L-arabinofurosyl-(1→2)-α-D-xylopyranosyl-(1→6)-β-D-glucopyranosyl-), T (α-L-arabino-furosyl-(1→3)-α-L-arabinofurosyl-(1→2)-α-D-xylopyranosyl-(1→6)-β-D-glucopyranosyl-), and J (α-L-galactopyranosyl-(1→2)-β-D-galactopyranosyl-(1→2)-α-D-xylopyranosyl-(1→6)-β-D-gluco-pyranosyl-) subunits according to the nomenclature of Fry et al. (Physiologia Plantarum 89: 1-3, 1993). In another aspect, the xyloglucan oligomer is the XXXG heptasaccharide common to dicotyledons and nongraminaceous monocots. In another aspect, the xyloglucan oligomer is O-acetylated. In another aspect, the xyloglucan oligomer is not O-acetylated. In another aspect, side chains of the xyloglucan oligomer may contain terminal fucosyl residues. In another aspect, side chains of the xyloglucan oligomer may contain terminal arabinosyl residues. In another aspect, side chains of the xyloglucan oligomer may contain terminal xylosyl residues.

### Functionalization of Xyloglucan Oligomer and Polymeric Xyloglucan

The xyloglucan oligomer can be functionalized by incorporating any chemical group known to those skilled in the art. The chemical group may be an agriculturally beneficial agent or a reactive group such as an aldehyde group, an amino group, an aromatic group, a carboxyl group, a halogen group, a hydroxyl group, a ketone group, a nitrile group, a nitro group, a sulfhydryl group, or a sulfonate group.

In one aspect, the chemical group is an aldehyde group.

In another aspect, the chemical group is an amino group. The amino group can be incorporated into polymeric xyloglucan by reductive amination. Alternatively, the amino group can be an aliphatic amine or an aromatic amine (e.g., aniline). The aliphatic amine can be a primary, secondary or tertiary amine. Primary, secondary, and tertiary amines are nitrogens bound to one, two and three carbons, respectively. In one aspect, the primary amine is C₁-C₈, e.g., ethylamine. In another aspect, each carbon in the secondary amine is C₁-C₈, e.g., diethylamine. In another aspect, each carbon in the tertiary amine is C₁-C₈, e.g., triethylamine.

In another aspect, the chemical group is an aromatic group. The aromatic group can be an arene group, an aryl halide group, a phenolic group, a phenylamine group, a diazonium group, or a heterocyclic group.

In another aspect, the chemical group is a carboxyl group. The carboxyl group can be an acyl halide, an amide, a carboxylic acid, an ester, or a thioester.

In another aspect, the chemical group is a halogen group. The halogen group can be fluorine, chlorine, bromine, or iodine.

In another aspect, the chemical group is a hydroxyl group.

In another aspect, the chemical group is a ketone group.

In another aspect, the chemical group is a nitrile group.

In another aspect, the chemical group is a nitro group.

In another aspect, the chemical group is a sulfhydryl group.

In another aspect, the chemical group is a sulfonate group.

The chemical reactive group can itself be the chemical group that imparts a desired physical or chemical property to an agricultural crop.

By incorporation of chemical reactive groups in such a manner, one skilled in the art can further derivatize the incorporated reactive groups with an agriculturally beneficial agent. For example, the incorporated chemical group may react with the compound that imparts the desired property to incorporate that group into the xyloglucan oligomer via a covalent bond. Alternatively, the chemical group may bind to the compound that imparts the desired property in either a reversible or irreversible manner, and incorporate the compound via a non-covalent association. The derivatization can be performed directly on the functionalized xyloglucan oligomer or after the functionalized xyloglucan oligomer is incorporated into polymeric xyloglucan.

Alternatively, the xyloglucan oligomer can be functionalized by incorporating directly an agriculturally beneficial agent by using a reactive group contained in the agriculturally beneficial agent or a reactive group incorporated into the agriculturally beneficial agent, such as any of the groups described above.

On the other hand, the polymeric xyloglucan can be directly functionalized by incorporating a reactive chemical group as described above. By incorporation of reactive chemical groups directly into polymeric xyloglucan, one of skill in the art can further derivatize the incorporated reactive groups with an agriculturally beneficial agent.

The incorporated reactive group can also result in modifying the polymeric xyloglucan so it is amenable to electrostatic or hydrophobic interaction with an agriculturally beneficial agent.

The incorporated chemical group can also result in modifying the polymeric xyloglucan so it is amenable to a specific binding interaction (e.g., antibody-antigen, avidin-biotin, protein-ligand, aptamer-ligand or the like) with an agriculturally beneficial agent.

In one aspect, the functionalization is performed by reacting the reducing end hydroxyl of the xyloglucan oligomer or the polymeric xyloglucan. In another aspect, a non-reducing hydroxyl group, other than the non-reducing hydroxyl at position 4 of the terminal glucose, can be reacted. In another aspect, the reducing end hydroxyl and a non-reducing hydroxyl, other than the non-reducing hydroxyl at position 4 of the terminal glucose, can be reacted.

The chemical functional group can be added by enzymatic modification of the xyloglucan oligomer or polymeric xyloglucan, or by a non-enzymatic chemical reaction. In one aspect, enzymatic modification is used to add the chemical functional group. In one embodiment of enzymatic modification, the enzymatic functionalization is oxidation to a ketone or carboxylate, e.g., by galactose oxidase. In another embodiment of enzymatic modification, the enzymatic functionalization is oxidation to a ketone or carboxylate by AA9 Family oxidases (formerly glycohydrolase Family 61 enzymes).

In another aspect, the chemical functional group is added by a non-enzymatic chemical reaction. In one embodiment of the non-enzymatic chemical reaction, the reaction is incorporation of a reactive amine group by reductive amination of the reducing end of the carbohydrate as described by Roy et al., 1984, Can. J. Chem. 62: 270-275, or Dalpathado et al., 2005, Anal. Bioanal. Chem. 381: 1130-1137. In another embodiment of non-enzymatic chemical reaction, the reaction is incorporation of a reactive ketone group by oxidation of the reducing end hydroxyl to a ketone, e.g., by copper (II). In another embodiment of non-enzymatic chemical reaction, the reaction is oxidation of non-reducing end hydroxyl groups (e.g., of the non-glycosidic bonded position 6 hydroxyls of glucose or galactose) by (2,2,6,6-tetramethyl-piperidin-1-yl)oxyl (TEMPO), or the oxoammonium salt thereof, to generate an aldehyde or carboxylic acid as described in Bragd et al., 2002, Carbohydrate Polymers 49: 397-406, or Breton et al., 2007, Eur. J. Org. Chem. 10: 1567-1570.

Xyloglucan oligomers or polymeric xyloglucan can be functionalized by a chemical reaction with agriculturally beneficial agents containing more than one (i.e. bifunctional or multifunctional) chemical functional group comprising at least one chemical functional group that is directly reactive with xyloglucan oligomer or polymeric xyloglucan. In one aspect, the bifunctional chemical group is a hydrocarbon containing a primary amine and a second chemical functional group. The second functional group can be any of the other groups described above.

Xyloglucan oligomers or polymeric xyloglucan can be functionalized with an agriculturally beneficial agent by step-wise or concerted reaction wherein the xyloglucan oligomer or polymeric xyloglucan is functionalized as described above, and the agriculturally beneficial agent is reactive to the functionalization introduced therein. In one aspect of coupling via a functionalized xyloglucan oligomer, an amino group is first incorporated into the xyloglucan oligomer by reductive amination and a reactive carbonyl is secondarily coupled to the introduced amino group. One logical example of this aspect is coupling of amine-functionalized xyloglucan to the herbicide glyphosate via the carboxyl moiety of the glyphosate. In another aspect of coupling via an amino-modified xyloglucan oligomer, the second coupling step incorporates a chemical group or an agriculturally beneficial agent via coupling an N-hydroxysuccinimidyl (NHS) ester or imidoester to the introduced amino group. In a preferred embodiment, the NHS ester secondarily coupled to the introduced amino group is a component of a mono or bi-functional crosslink reagent. In another aspect of coupling to a functionalized xyloglucan or xyloglucan oligomer, the first reaction step comprises functionalization with a sulfhydryl group, either via reductive amination with an alkylthioamine (NH₂—(CH₂)ₙ—SH) at elevated temperatures in the presence of a reducing agent (Magid et al., 1996, J. Org. Chem. 61: 3849-3862), or via radical coupling (Wang et al., 2009, Arkivoc xiv: 171-180), followed by reaction of a maleimide group to the sulfhydryl. In some aspects, the reactive group in the compound that imparts the desired property is separated from the rest of the compound by a hydrocarbon chain of an appropriate length, as is well described in the art.

Non-limiting examples of agriculturally beneficial agents that can be used as functional groups for polymeric xyloglucan or xyloglucan oligomers, either by direct reaction or via reaction with a xyloglucan-reactive compound, include fungicides, herbicides, insecticides, nematode antagonistic agents, acaricides, beneficial microorganisms, plant signal molecules, nutrients, biostimulants, preservatives, polymers, wetting agents, surfactants, or combinations thereof.

### Agriculturally Beneficial Agents

The formulations disclosed herein comprise one or more agriculturally beneficial agents each at an effective dosage to impart an agricultural benefit. Non-limiting examples of agriculturally beneficial agents include one or more fungicides, herbicides, insecticides, nematode antagonistic agents, acaricides, beneficial microorganisms, plant signal molecules, nutrients, biostimulants, preservatives, polymers, wetting agents, surfactants, anti-freezing agents, minerals, microbially stabilizing compounds, or combinations thereof.

In one aspect, the agriculturally beneficial agent is a fungicide. In another aspect, the agriculturally beneficial agent is a herbicide. In another aspect, the agriculturally beneficial agent is an insecticide. In another aspect, the agriculturally beneficial agent is a nematode antagonistic agent. In another aspect, the agriculturally beneficial agent is an acaricide. In another aspect, the agriculturally beneficial agent is a beneficial microorganism. In another aspect, the agriculturally beneficial agent is a plant signal molecule. In another aspect, the agriculturally beneficial agent is a nutrient. In another aspect, the agriculturally beneficial agent is a biostimulant. In another aspect, the agriculturally beneficial agent is a preservative. In another aspect, the agriculturally beneficial agent is a polymer. In another aspect, the agriculturally beneficial agent is a wetting agent. In another aspect, the agriculturally beneficial agent is a surfactant. In another aspect, the agriculturally beneficial agent is an anti-freezing agent. In another aspect, the agriculturally beneficial agent is a mineral. In another aspect, the agriculturally beneficial agent is a microbially stabilizing compound.

Fungicides: The formulations described herein may comprise or further comprise one or more fungicides. Fungicides useful in the formulations described herein may be chemical fungicides, biological fungicides, or combinations thereof. Fungicides may be selected so as to provide effective control against a broad spectrum of phytopathogenic fungi, including soil-borne fungi, which derive especially from the classes of the Plasmodiophoromycetes, Peronosporomycetes (syn. Oomycetes), Chytridiomycetes, Zygomycetes, Ascomycetes, Basidiomycetes, and Deuteromycetes (syn. Fungi imperfecti). More common fungal pathogens that may be targeted include Fusarium, Phakopsora, Phomopsis, Pythium, Pytophthora, Rhizoctonia, or Selerotinia, and combinations thereof.

Fungicide classes include ACCase inhibitors, aromatic hydrocarbons, benzimidazoles, benzothiadiazole, carboxylic acid amides, phenylamides, phosphonates, quinone outside inhibitors (which embrace strobilurins), thiazolidines, thiophanates, thiophene carboxamides, and triazoles. Particular examples of fungicides include carbendazim, fosetyl-AI, thiophanate, and tolclofos-methyl.

Examples of chemical fungicides include at least one member selected from the group consisting of azoles, carboxamides, heterocyclic compounds, strobilurins, and other active substances.

Examples of azoles as fungicides include, but are not limited to, azaconazole, bitertanol, bromuconazole, cyproconazole, difenoconazole, diniconazole, diniconazole-M, epoxiconazole, fenbuconazole, fluquinconazole, flusilazole, flutriafol, hexaconazole, imibenconazole, ipconazole, metconazole, myclobutanil, oxpoconazole, paclobutrazole, penconazole, propiconazole, prothioconazole, simeconazole, tebuconazole, tetraconazole, triadimefon, triadimenol, triticonazole, uniconazole; imidazoles: cyazofamid, imazalil, pefurazoate, prochloraz, and triflumizol.

Examples of carboxamides as fungicides include, but are not limited to, benalaxyl, benalaxyl-M, benodanil, bixafen, boscalid, carboxin, fenfuram, fenhexamid, flutolanil, fluxapyroxad, furametpyr, isopyrazam, isotianil, kiralaxyl, mepronil, metalaxyl, metalaxyl-M (mefenoxam), ofu race, oxadixyl, oxycarboxin, penflufen, penthiopyrad, sedaxane, tecloftalam, thifluzamide, tiadinil, 2-amino-4-methyl-thiazole-5-carboxanilide, N-(4′-trifluoromethylthiobiphenyl-2-yl)-3-difluoromethyl-1-methyl-1H-pyrazole-4-carboxamide and N-(2-(1,3,3-trimethylbutyl)-phenyl)-1,3-dimethyl-5-fluoro-1H-pyrazole-4-carboxamide; carboxylic morpholides: dimethomorph, flumorph, pyrimorph; benzoic acid amides: flumetover, fluopicolide, fluopyram, zoxamide; carpropamid, dicyclomet, mandiproamid, oxytetracyclin, silthiofam, and N-(6-methoxy-pyridin-3-yl) cyclopropanecarboxylic acid amide.

Examples of heterocyclic compounds as fungicides include, but are not limited to, fluazinam, pyrifenox, 3-[5-(4-chloro-phenyl)-2,3-dimethyl-isoxazolidin-3-yl]-pyridine, 3-[5-(4-methyl-phenyl)-2,3-dimethyl-isoxazolidin-3-yl]-pyridine; pyrimidines: bupirimate, cyprodinil, diflumetorim, fenarimol, ferimzone, mepanipyrim, nitrapyrin, nuarimol, pyrimethanil;

piperazines: triforine; pyrroles: fenpiclonil, fludioxonil; morpholines: aldimorph, dodemorph, dodemorph-acetate, fenpropimorph, tridemorph; piperidines: fenpropidin; dicarboximides: fluoroimid, iprodione, procymidone, vinclozolin; non-aromatic 5-membered heterocycles: famoxadone, fenamidone, flutianil, octhilinone, probenazole, 5-amino-2-isopropyl-3-oxo-4-ortho-tolyl-2,3-dihydro-pyrazole-1-carbothioic acid S-allyl ester; others: acibenzolar-S-methyl, ametoctradin, amisulbrom, anilazin, blasticidin-S, captafol, captan, chinomethionat, dazomet, debacarb, diclomezine, difenzoquat, difenzoquat-methylsulfate, fenoxanil, N-[(trichloromethyl)thio]phtalimide, oxolinic acid, piperalin, proquinazid, pyroquilon, quinoxyfen, triazoxide, tricyclazole, 2-butoxy-6-iodo-3-propylchromen-4-one, 5-chloro-1-(4,6-dimethoxy-pyrimidin-2-yl)-2-methyl-1H-benzoimidazole, and 5-chloro-7-(4-methylpiperidin-1-yl)-6-(2,4,6-trifluorophenyl)-[1,2,4]triazolo-[1,5-a]pyrimidine.

Examples of strobilurins as fungicides include, but are not limited to, azoxystrobin, coumethoxystrobin, coumoxystrobin, dimoxystrobin, enestroburin, fluoxastrobin, kresoxim-methyl, metominostrobin, orysastrobin, picoxystrobin, pyraclostrobin, pyrametostrobin, pyraoxystrobin, pyribencarb, trifloxystrobin, 2-[2-(2,5-dimethyl-phenoxymethyl)-phenyl]-3-methoxy-acrylic acid methyl ester and 2-(2-(3-(2,6-dichlorophenyl)-1-methyl-allylideneaminooxymethyl)-phenyl)-2-methoxyimino-N-methyl-acetamide.

Examples of other active substances as fungicides include, but are not limited to, fluazinam, pyrifenox, 3-[5-(4-chloro-phenyl)-2,3-dimethyl-isoxazolidin-3-yl]-pyridine, 3-[5-(4-methyl-phenyl)-2,3-dimethyl-isoxazolidin-3-yl]-pyridine; pyrimidines: bupirimate, cyprodinil, diflumetorim, fenarimol, ferimzone, mepanipyrim, nitrapyrin, nuarimol, pyrimethanil; piperazines: triforine; pyrroles: fenpiclonil, fludioxonil; fuberidazole, mancozeb, morpholines: aldimorph, dodemorph, dodemorph-acetate, fenpropimorph, tridemorph; piperidines: fenpropidin; dicarboximides: fluoroimid, iprodione, procymidone, vinclozolin; non-aromatic 5-membered heterocycles: famoxadone, fenamidone, flutianil, octhilinone, probenazole, 5-amino-2-isopropyl-3-oxo-4-ortho-tolyl-2,3-dihydro-pyrazole-1-carbothioic acid S-allyl ester; SYP-1620, SYP-Z048, thiabendazole, thiophanate-methyl, thiram, acibenzolar-S-methyl, ametoctradin, amisulbrom, anilazin, blasticidin-S, captafol, captan, chinomethionat, dazomet, debacarb, diclomezine, difenzoquat, difenzoquat-methylsulfate, fenoxanil, N-[(trichloromethyl)thio]phtalimide, oxolinic acid, piperalin, proquinazid, pyroquilon, and quinoxyfen.

Examples of biological fungicides include, but are not limited to, Ampelomyces quisqualis (e.g., AQ 10® from Intrachem Bio GmbH & Co. KG, Germany), Aspergillus flavus (e.g., AFLAGUARD® from Syngenta, CH), Aureobasidium pullulans (e.g., BOTECTOR® from bio-ferm GmbH, Germany), Bacillus pumilius (e.g., isolate NRRL-Nr. B-21661 in RHAPSODY®, SERENADE® MAX and SERENADE® ASO from Fa. AgraQuest Inc., USA), Bacillus subtilis var. amyloliquefaciens FZB24 (e.g., TAEGRO® from Novozyme Biologicals, Inc., USA), Candida oleophila 1-82 (e.g., ASPIRE® from Ecogen Inc., USA), Candida saitoana (e.g., BIOCURE® in a mixture with lysozyme, BIOCOAT® from Micro Flo Company, USA (BASF SE), and Arysta), Chitosan (e.g., ARMOUR-ZEN from BotriZen Ltd., NZ), Clonostachys rosea f. catenulata, also named Gliocladium catenulatum (e.g., isolate J1446: PRESTOP® from Verdera, Finland), Coniothyrium minitans (e.g., CONTANS® from Prophyta, Germany), Ctyphonectria parasitica (e.g., Endothia parasitica from CNICM, France), Cryptococcus albidus (e.g., YIELD PLUS® from Anchor Bio-Technologies, South Africa), Fusarium oxysporum (e.g., BIOFOX® from S.I.A.P.A., Italy, FUSACLEAN® from Natural Plant Protection, France), Metschnikowia fructicola (e.g., SHEMER® from Agrogreen, Israel), Microdochium dimerum (e.g., ANTIBOT® from Agrauxine, France), Phlebiopsis gigantea (e.g., ROTSOP® from Verdera, Finland), Pseudozyma flocculosa (e.g., SPORODEX® from Plant Products Co. Ltd., Canada), Pythium oligandrum DV74 (e.g., POLYVERSUM® from Remeslo SSRO, Biopreparaty, Czech Rep.), Reynoutria sachlinensis (e.g., REGALIA® from Marrone BioInnovations, USA), Talaromyces flavus V117b (e.g., PROTUS® from Prophyta, Germany), Trichoderma asperellum SKT-1 (e.g., ECO-HOPE® from Kumiai Chemical Industry Co., Ltd., Japan), T. atroviride LC52 (e.g., SENTINEL® from Agrimm Technologies Ltd, NZ), T. harzianum T-22 (e.g., PLANTSHIELD® der Firma BioWorks Inc., USA), T. harzianum TH 35 (e.g., ROOT PRO® from Mycontrol Ltd., Israel), T. harzianum T-39 (e.g., TRICHODEX® and TRICHODERMA 2000® from Mycontrol Ltd., Israel and Makhteshim Ltd., Israel), T. harzianum and T. viride (e.g., TRICHOPEL from Agrimm Technologies Ltd, NZ), T. harzianum ICC012 and T. viride ICC080 (e.g., REMEDIER® WP from lsagro Ricerca, Italy), T. polysporum and T. harzianum (e.g., BINAB® from BINAB Bio-Innovation AB, Sweden), T. stromaticum (e.g., TRICOVAB® from C.E.P.L.A.C., Brazil), T. virens GL-21 (e.g., SOILGARD® from Certis LLC, USA), T. viride (e.g., TRIECO® from Ecosense Labslndiaa Pvt. Ltd., Mumbai, India BIO-CURE® F from T. Stanes & Co. Ltd., Tamil Nadu, India), T. viride TV1 (e.g., T. viride TV1 from Agribiotec srl, Italy), Ulocladium oudemansii HRU3 (e.g., BOTRY-ZEN® from Botry-Zen Ltd, NZ) and chitosan (e.g., ARMOUR-ZEN from BotriZen Ltd., NZ).

Herbicides: The formulations described herein may comprise or further comprise one or more herbicides. Suitable herbicides include chemical herbicides, natural herbicides (e.g., bioherbicides, organic herbicides, etc.), or combinations thereof. Herbicides useful in the formulations described herein may include at least one member selected from the group consisting of a pre-emergent herbicide, a post-emergent herbicide, or a combination thereof. Non-limiting examples of suitable herbicides include ACCase inhibitors, acetanilides, acetochlor, acifluorfen, AHAS inhibitors, bentazon, carotenoid biosynthesis inhibitors, clethodim, chlorimuron, clomazone, 2,4-D, dicamba, EPSPS inhibitors, fluazifop, flumiclorac, flumioxazin, fomesafen, glufosinate, glutamine synthetase inhibitors, glyphosate, imazamox, imazaquin, imazethapyr, lactofen, mesotrione, PPO inhibitors, PS II inhibitors, quizalofop, saflufenacil, sethoxydim, sulcotrione, and synthetic auxins. Commercial products containing each of these compounds are readily available. Herbicide concentration in the formulation will generally correspond to the labeled use rate for a particular herbicide. Insecticides: The formulations described herein may comprise or further comprise one or more insecticides. Insecticides useful in the formulations described herein include at least one member selected from the group consisting of acetamiprid, betacyfluthrin, clothianidin, cyantraniliprole, diafenthiuron, diazinon, emamectin (benzoate), fenoxycarb, fipronil, flonicamid, imidacloprid, lambda-cyhalothrin, lufenuron, methiocarb, pymetrozine, pyrifluquinazon, pyriproxyfen, spinetoram, spinosad, spirotetramat, tefluthrin, thiacloprid, thiamethoxam, thiodicarb, and Ti-435. Examples of other insecticides include diamides, macrocyclic lactones, neonicotinoids, organophosphates, phenylpyrazoles, pyrethrins, spinosyns, synthetic pyrethroids, tetronic acids, and tetramic acids. In particular embodiments, insecticides include bifenthrin, chlorantraniliporle, chlothianidin, cyfluthrin, cyhalothrin, cypermethrin, deltamethrin, dinotefuran, ethiprole, flubendiamide, milbemectin, nitenpyram, spirodichlofen, and tioxazafen.

Nematode-antagonistic agents: The formulations described herein may comprise or further comprise one or more nematode-antagonistic agents. Nematode-antagonistic agents useful in the formulations described herein include nematicides, nematophagous fungi, and nematophagous bacteria.

Examples of nematicides include, but are not limited to, at least one member selected from the group consisting of avermectin nematicides, such as abamectin; carbamate nematicides, such as alanycarb, aldicarb, aldoxycarb, benomyl, carbofuran, carbosulfan, oxamyl, ethoprop, and methomyl; and organophosphorus nematicides, such as cadusafos, chlorpyrifos, diamidafos, dichlofenthion, dimethoate, fenamiphos, fensulfothion, fosthiazate, fosthietan, heterophos, isamidofos, isazofos ethoprophos, mecarphon, phosphamidon, phorate, phosphocarb, terbufos, thionazin, and triazophos. Examples of other nematicides include diamides, macrocyclic lactones, neonicotinoids, organophosphates, phenylpyrazoles, pyrethrins, spinosyns, synthetic pyrethroids, tetronic acids, and tetramic acids. In particular embodiments, nematicides include bifenthrin, chlorantraniliporle, chlothianidin, cyfluthrin, cyhalothrin, cypermethrin, deltamethrin, dinotefuran, ethiprole, flubendiamide, milbemectin, nitenpyram, spirodichlofen, and tioxazafen.

Examples of nematophagous fungi include, but are not limited to, Arthrobotrys spp., for example, Arthrobotrys oligospora, Arthrobotrys superb, and Arthrobotrys dactyloides; Dactylaria spp., for example, Dactylaria candida; Harposporium spp., for example, Harposporium anguillulae; Hirsutella spp., for example, Hirsutella rhossiliensis and Hirsutella minnesotensis, Monacrosporium spp., for example, Monacrosporium cionopagum; Nematoctonus spp., for example, Nematoctonus geogenius, Nematoctonus leiosporus; Meristacrum spp., for example, Meristacrum asterospermum; spp., for example, Paecilomyces lilacinus; Pochonia spp., for example, Pochonia chlamydopora, and Streptomyces spp.

Examples of nematophagous bacteria include, but are not limited to, obligate parasitic bacteria, opportunistic parasitic bacteria, rhizobacteria, parasporal Cry protein-forming bacteria, endophytic bacteria and symbiotic bacteria. In particular embodiments, the biocontrol agent can be a bacteria species selected from Actinomycetes spp., Agrobacterium spp., Allorhizobium spp., Alcaligenes spp., Arthrobacter spp., Aureobacterium spp., Azobacter spp., Azorhizobium spp., Azospirillium spp., Befierinckia spp., Bradyrhizobium spp., Burkholderia spp., Chromobacterium spp., Clavibacter spp., Clostridium spp., Comomonas spp., Corynebacterium spp., Curtobacterium spp., Desulforibtio spp., Enterobacter spp., Flavobacterium spp., Gluconobacter spp., Hydrogenophage spp., Klebsiella spp., Methylobacterium spp., Paenibacillus spp., Pasteuria spp., Phingobacterium spp., Photorhabdus spp., Pseudomonas spp., Phyllobacterium spp., Rhizobium spp., Serratia spp., Stenotrotrophomonas spp., Variovorax spp., and Xenorhadbus spp.

Preferred nematode-antagonistic agents include ARF18, Arthrobotrys spp., Brevibacillus spp., Burkholderia spp., Chaetomium spp., Cylindrocarpon spp., Exophilia spp., Fusarium spp., Gliocladium spp., Hirsutella spp., Lecanicillium spp., Monacrosporium spp., Myrothecium spp., Neocosmospora spp., Paecilomyces spp., Pochonia spp., Stagonospora spp., Pasteuria spp., Pseudomonas spp., and Rhizobacteria.

Acaricides: The formulations described herein may comprise or further comprise one or more acaricides. Acaricides useful in the formulations described herein include at least one member selected from the group consisting of antibiotic miticides, carbamate miticides, diatomaceous earth, dicofol, formamidine miticides, ivermectin, lime sulfur, organophosphosphate miticides, and permethrin.

Beneficial Microorganisms: The formulations described herein may comprise or further comprise one or more beneficial microorganisms. The beneficial microorganisms useful in the formulations described herein may include any number of microorganisms having one or more beneficial properties (e.g., pesticidal properties, produce one or more of the plant signal molecules described herein, enhance nutrient and water uptake, promote and/or enhance nitrogen fixation, enhance growth, enhance seed germination, enhance seedling emergence, break the dormancy or quiescence of a plant, provide anti-fungal activity, etc.). The beneficial microorganisms useful in the formulations described herein may be in a spore form, a vegetative form, or a combination thereof. The formulations comprising or further comprising one or more beneficial microorganisms may serve as inoculums.

In one embodiment, the one or more beneficial microorganisms are diazotrophs (i.e., bacteria which are symbiotic nitrogen-fixing bacteria). In another embodiment, the one or more beneficial microorganisms are bacteria selected from the genera Bradyrhizobium spp., Azorhizobium spp., Azospirillum spp., Sinorhizobium spp., Mesorhizobium spp., Rhizobium spp., and combinations thereof. In another embodiment, the one or more beneficial microorganisms are bacteria selected from the group consisting of Azorhizobium caulinodans, Azorhizobium doebereinerae, Azospirillum amazonense, Azospirillum brasilense, Azospirillum canadense, Azospirillum doebereinerae, Azospirillum formosense, Azospirillum halopraeferans, Azospirillum irakense, Azospirillum largimobile, Azospirillum lipoferum, Azospirillum melinis, Azospirillum otyzae, Azospirillum picis, Azospirillum rugosum, Azospirillum thiophilum, Azospirillum zeae, Bradyrhizobium bete, Bradyrhizobium canariense, Bradyrhizobium elkanii, Bradyrhizobium iriomotense, Bradyrhizobium japonicum, Bradyrhizobium jicamae, Bradyrhizobium liaoningense, Bradyrhizobium pachyrhizi, Bradyrhizobium yuanmingense, Mesorhizobium albiziae, Mesorhizobium amorphae, Mesorhizobium chacoense, Mesorhizobium ciceri, Mesorhizobium huakuii, Mesorhizobium loti, Mesorhizobium mediterraneum, Mesorhizobium pluifarium, Mesorhizobium septentrionale, Mesorhizobium ternperatum, Mesorhizobium tianshanense, Rhizobium cellulosilyticum, Rhizobium daejeonense, Rhizobium etli, Rhizobium gale gae, Rhizobium gallicum, Rhizobium giardinii, Rhizobium hainanense, Rhizobium huautlense, Rhizobium indigo ferae, Rhizobium leguminosarum, Rhizobium loessense, Rhizobium lupini, Rhizobium lusitanum, Rhizobium meliloti, Rhizobium mongolense, Rhizobium miluonense, Rhizobium sullae, Rhizobium tropici, Rhizobium undicola, Rhizobium yanglingense, Sinorhizobium abri, Sinorhizobium adhaerens, Sinorhizobium americanurn, Sinorhizobium aboris Sinorhizobium fredii, Sinorhizobium indiaense, Sinorhizobium kostiense, Sinorhizobium kummerowiae, Sinorhizobium medicae, Sinorhizobium meliloti, Sinorhizobium mexicanus, Sinorhizobium morelense, Sinorhizobium saheli, Sinorhizobium terangae, Sinorhizobium xinjiangense, and combinations thereof.

In a particular embodiment, the beneficial microorganism is selected from the group consisting of Azospirillum brasilense, Bradyrhizobium japonicum, Rhizobium leguminosarum, Rhizobium meliloti, Sinorhizobium meliloti, and combinations thereof. In another embodiment, the beneficial microorganism is Azospirillum brasilense. In another embodiment, the beneficial microorganism is Bradyrhizobium japonicum. In another embodiment, the beneficial microorganism is Rhizobium leguminosarum. In another embodiment, the beneficial microorganism is Rhizobium meliloti. In another embodiment, the beneficial microorganism is Sinorhizobium meliloti.

In another embodiment, the beneficial microorganism is selected from the group consisting of Bacillus cereus, Bacillus, lichen formis, Bacillus sphaericus, Chromobacterium suttsuga, Pasteuria penetrans, Pasteuria usage, and Pseudomona fluorescens.

In another embodiment, the beneficial microorganism is of the genus selected from the group consisting of Alternaria, Beauveria, Colletotrichum, Gliocladium, Metarhisium, Muscodor, Paecilomyces, Trichoderma, Typhula, and Verticilium. In particular embodiments the fungus is Beauveria bassiana, Gliocladium virens, Muscodor albus, or Trichoderma polysporum.

In another embodiment, the one or more beneficial microorganisms comprise one or more phosphate solubilizing microorganisms. Phosphate solubilizing microorganisms include fungal and bacterial strains. In an embodiment, the phosphate solubilizing microorganism are microorganisms selected from the genera consisting of Acinetobacter spp., Arthrobacter spp, Arthrobotrys spp., Aspergillus spp., Azospirillum spp., Bacillus spp., Burkholderia spp., Candida spp., Chtyseomonas spp., Enterobacter spp., Eupenicillium spp., Exiguobacterium spp., Klebsiella spp., Kluyvera spp., Microbacterium spp., Mucor spp., Paecilomyces spp., Paenibacillus spp., Penicillium spp., Pseudomonas spp., Serratia spp., Stenotrophomonas spp., Streptomyces spp., Streptosporangium spp., Swaminathania spp., Thiobacillus spp., Torulospora spp., Vibrio spp., Xanthobacter spp., Xanthomonas spp., and combinations thereof. In still yet another embodiment, the phosphate solubilizing microorganism is a microorganism selected from the group consisting of Acinetobacter calcoaceticus, Arthrobottys oligospora, Aspergillus niger, Azospirillum amazonense, Azospirillum brasilense, Azospirillum canadense, Azospirillum doebereinerae, Azospirillum formosense, Azospirillum halopraeferans, Azospirillum irakense, Azospirillum largimobile, Azospirillum lipoferum, Azospirillum melinis, Azospirillum otyzae, Azospirillum picis, Azospirillum rugosum, Azospirillum thiophilum, Azospirillum zeae, Bacillus amyloliquefaciens, Bacillus atrophaeus, Bacillus circulans, Bacillus licheniformis, Bacillus subtilis, Burkholderia cepacia, Burkholderia vietnamiensis, Candida krissii, Chtyseomonas luteola, Enterobacter aerogenes, Enterobacter asburiae, Enterobacter taylorae, Eupenicillium parvum, Kluyvera ctyocrescens, Mucor ramosissimus, Paecilomyces hepialid, Paecilomyces marquandii, Paenibacillus macerans, Paenibacillus mucilaginosus, Penicillium bilaiae (formerly known as Penicillium bilaii), Penicillium albidum, Penicillium aurantiogriseum, Penicillium chtysogenum, Penicillium citreonigrum, Penicillium citrinum, Penicillium digitatum, Penicillium frequentas, Penicillium fuscum, Penicillium gaestrivorus, Penicillium glabrum, Penicillium griseofulvum, Penicillium implicatum, Penicillium janthinellum, Penicillium lilacinum, Penicillium minioluteum, Penicillium montanense, Penicillium nigricans, Penicillium oxalicum, Penicillium pinetorum, Penicillium pinophilum, Penicillium purpurogenum, Penicillium radicans, Penicillium radicum, Penicillium raistrickii, Penicillium rugulosum, Penicillium simplicissimum, Penicillium solitum, Penicillium variabile, Penicillium velutinum, Penicillium viridicatutn, Penicillium glaucum, Penicillium fussiporus, and Penicillium expansum, Pseudomonas corrugate, Pseudomonas fluorescens, Pseudomonas lutea, Pseudomonas poae, Pseudomonas putida, Pseudomonas stutzeri, Pseudomonas trivialis, Serratia marcescens, Stenotrophomonas maltophilia, Swaminathania salitolerans, Thiobacillus ferrooxidans, Torulospora globose, Vibrio proteolyticus, Xanthobacter agilis, Xanthomonas campestris, and combinations thereof.

In a particular embodiment, the one or more phosphate solubilizing microorganisms are a strain of the fungus Penicillium. In another embodiment, the one or more Penicillium species are P. bilaiae, P. gaestrivorus, or combinations thereof.

In another embodiment, the beneficial microorganism is one or more mycorrhiza. In particular, the one or more mycorrhiza are an endomycorrhiza (also called vesicular arbuscular mycorrhizas or VAMs, or arbuscular mycorrhizas or AMs), an ectomycorrhiza, or a combination thereof.

In another embodiment, the one or more mycorrhiza are an endomycorrhiza of the phylum Glomeromycota and genera Glomus and Gigaspora. In still a further embodiment, the endomycorrhiza is a strain of Glomus aggregatum, Glomus brasilianum, Glomus clarum, Glomus deserticola, Glomus etunicatum, Glomus fasciculatum, Glomus intraradices, Glomus monosporum, or Glomus mosseae, Gigaspora margarita, or a combination thereof.

In another embodiment, the one or more mycorrhiza are an ectomycorrhiza of the phylum Ascomycota, Basidiomycota, and Zygomycota. In still yet another embodiment, the ectomycorrhiza is a strain of Laccaria bicolor, Laccaria laccata, Pisolithus tinctorius, Rhizopogon amylopogon, Rhizopogon fulvigleba, Rhizopogon luteolus, Rhizopogon villosuli, Scleroderma cepa, Scleroderma citrinum, or a combination thereof.

In another embodiment, the one or more mycorrhiza are selected from the group consisting of an ecroid mycorrhiza, an arbutoid mycorrhiza, and a monotropoid mycorrhiza. Arbuscular and ectomycorrhizas form ericoid mycorrhiza with many plants belonging to the order Ericales, while some Ericales form arbutoid and monotropoid mycorrhizas. All orchids are mycoheterotrophic at some stage during their lifecycle and form orchid mycorrhizas with a range of basidiomycete fungi. In one embodiment, the mycorrhiza may be an ericoid mycorrhiza, preferably of the phylum Ascomycota, such as Hymenoscyphous ericae or Oidiodendron sp. In another embodiment, the mycorrhiza also may be an arbutoid mycorrhiza, preferably of the phylum Basidiomycota. In yet another embodiment, the mycorrhiza may be a monotripoid mycorrhiza, preferably of the phylum Basidiomycota. In still yet another embodiment, the mycorrhiza may be an orchid mycorrhiza, preferably of the genus Rhizoctonia.

In another embodiment, the one or more beneficial microorganisms are fungicides, i.e., fungicidal activity, (e.g., biofungicides). Non-limiting examples of biofungicides include, Ampelomyces quisqualis (e.g., AQ 10® from Intrachem Bio GmbH & Co. KG, Germany), Aspergillus flavus (e.g., AFLAGUARD® from Syngenta, CH), Aureobasidium pullulans (e.g., BOTECTOR® from bio-ferm GmbH, Germany), Bacillus pumilius (e.g., isolate NRRL-Nr. B-21661 in RHAPSODY®, SERENADE® MAX and SERENADE® ASO from AgraQuest Inc., USA), Bacillus subtilis var. amyloliquefaciens FZB24 (e.g., TAEGRO® from Novozymes Biologicals, Inc., USA), Candida oleophila 1-82 (e.g., ASPIRE® from Ecogen Inc., USA), Candida saitoana (e.g., BIOCURE® in a mixture with lysozyme, BIOCOAT® from Micro Flo Company, USA (BASF SE), and Arysta), Clonostachys rosea f. catenulata, also named Gliocladium catenulatum (e.g., isolate J1446: PRESTOP® from Verdera, Finland), Coniothyrium minitans (e.g., CONTANS® from Prophyta, Germany), Cryphonectria parasitica (e.g., Endothia parasitica from CNICM, France), Cryptococcus albidus (e.g., YIELD PLUS® from Anchor Bio-Technologies, South Africa), Fusarium oxysporum (e.g., BIOFOX® from S.I.A.P.A., Italy, FUSACLEAN® from Natural Plant Protection, France), Metschnikowia fructicola (e.g., SHEMER® from Agrogreen, Israel), Microdochium dimerum (e.g., ANTIBOT® from Agrauxine, France), Phlebiopsis gigantea (e.g., ROTSOP® from Verdera, Finland), Pseudozyma flocculosa (e.g., SPORODEX® from Plant Products Co. Ltd., Canada), Pythium oligandrum DV74 (e.g., POLYVERSUM® from Remeslo SSRO, Biopreparaty, Czech Rep.), Reynoutria sachlinensis (e.g., REGALIA® from Marrone BioInnovations, USA), Talaromyces flavus V117b (e.g., PROTUS® from Prophyta, Germany), Trichoderma asperellum SKT-1 (e.g., ECO-HOPE® from Kumiai Chemical Industry Co., Ltd., Japan), T. atroviride LC52 (e.g., SENTINEL® from Agrimm Technologies Ltd, NZ), T. harzianum T-22 (e.g., PLANTSHIELD® der Firma BioWorks Inc., USA), T. harzianum TH 35 (e.g., ROOT PRO® from Mycontrol Ltd., Israel), T. harzianum T-39 (e.g., TRICHODEX® and TRICHODERMA 2000® from Mycontrol Ltd., Israel and Makhteshim Ltd., Israel), T. harzianum and T. viride (e.g., TRICHOPEL from Agrimm Technologies Ltd, NZ), T. harzianum ICC012 and T. viride ICC080 (e.g., REMEDIER® WP from lsagro Ricerca, Italy), T. polysporum and T. harzianum (e.g., BINAB® from BINAB Bio-Innovation AB, Sweden), T. stromaticum (e.g., TRICOVAB® from C.E.P.L.A.C., Brazil), T. virens GL-21 (e.g., SOILGARD® from Certis LLC, USA), T. viride (e.g., TRIECO® from Ecosense LabsIndiaa Pvt. Ltd., Mumbai, India, BIO-CURE® F from T. Stanes & Co. Ltd., Tamil Nadu, India), T. viride TV1 (e.g., T. viride TV1 from Agribiotec srl, Italy), Ulocladium oudemansii HRU3 (e.g., BOTRY-ZEN® from Botry-Zen Ltd, NZ) and chitosan (e.g., ARMOUR-ZEN from BotriZen Ltd., NZ).

In a particular embodiment, the biofungicide is Bacillus subtilis var. amyloliquefaciens FZB24 (e.g., TAEGRO® from Novozymes Biologicals, Inc., USA).

Plant Signal Molecules:

The formulations described herein may comprise or further comprise one or more plant signal molecules. Plant signal molecules useful in the formulations described herein include, but are not limited to, lipo-chitooligosaccharides (LCOs), chitooligosaccharides (COs), chitinous compounds, jasmonic acid or derivatives thereof, linoleic acid or derivatives thereof, linolenic acid or derivatives thereof, non-flavonoid nod-gene inducers, karrikins, etc.

In one embodiment, the one or more plant signal molecules are one or more LCOs. In another embodiment, the one or more plant signal molecules are one or more COs. In another embodiment, the one or more plant signal molecules are one or more chitinous compounds. In another embodiment, the one or more plant signal molecules are one or more flavonoid or non-flavonoid nod gene inducers (e.g., jasmonic acid, linoleic acid, linolenic acid, and derivatives thereof). In another embodiment, the one or more plant signal molecules are one or more karrikins or derivatives thereof. In another embodiment, the one or more plant signal molecules are one or more LCOs, one or more COs, one or more chitinous compounds, one or more non-flavonoid nod gene inducers and derivatives thereof, one or more karrikins and derivatives thereof, or any signal molecule combination thereof.

Lipo-chitooligosaccharides. The formulations described herein may comprise one or more lipo-chitooligosaccharide compounds (LCOs) as plant signal molecules. LCOs, also known in the art as symbiotic Nod signals or Nod factors, consist of an oligosaccharide backbone of β-I,4-linked N-acetyl-D-glucosamine (“GlcNAc”) residues with an N-linked fatty acyl chain condensed at the non-reducing end. LCOs differ in the number of GlcNAc residues in the backbone, in the length and degree of saturation of the fatty acyl chain, and in the substitutions of reducing and non-reducing sugar residues. LCOs are intended to include all LCOs as well as isomers, salts, and solvates thereof.

LCOs may be obtained (isolated and/or purified) from bacteria such as Rhizobia, e.g., Rhizobium spp., Bradyrhizobium spp., Sinorhizobium spp., and Azorhizobium spp. The LCO structure is characteristic for each such bacterial species, and each strain may produce multiple LCOs with different structures. For example, specific LCOs from S. meliloti have also been described in U.S. Pat. No. 5,549,718.

Even more specific LCOs include NodRM, NodRM-1, NodRM-3. When acetylated (the R═CH₃CO—), they become AcNodRM, AcNodRM-1, and AcNodRM-3, respectively (U.S. Pat. No. 5,545,718).

LCOs from Bradyrhizobium japonicum are described in U.S. Pat. Nos. 5,175,149 and 5,321,011. Broadly, they are pentasaccharide phytohormones comprising methylfucose. A number of these B. japonicum-derived LCOs are described: BjNod-V (C_{18:1}); BjNod-V (A_{C}, C_{18:1}), BjNod-V (C_{16:1}); and BjNod-V (A_{C}, C_{16:0}), with “V” indicating the presence of five N-acetylglucosamines; “Ac” an acetylation; the number following the “C” indicating the number of carbons in the fatty acid side chain; and the number following the “:” the number of double bonds.

LCOs used in formulations of the invention may be obtained (i.e., isolated and/or purified) from bacterial strains that produce LCOs, such as strains of Azorhizobium, Bradyrhizobium (including B. japonicum), Mesorhizobium, Rhizobium (including R. leguminosarum), Sinorhizobium (including S. meliloti), and bacterial strains genetically engineered to produce LCOs.

Also encompassed by the formulations of the present invention are formulations using LCOs obtained (i.e., isolated and/or purified) from a mycorrhizal fungus, such as fungi of the group Glomerocycota, e.g., Glomus intraradicus. The structures of representative LCOs obtained from these fungi are described in WO 2010/049751 and WO 2010/049751 (the LCOs described therein are also referred to as “Myc factors”).

Further encompassed by the formulations of the present invention is use of synthetic LCO compounds, such as those described in WO 2005/063784, and recombinant LCOs produced through genetic engineering. The basic, naturally occurring LCO structure may contain modifications or substitutions found in naturally occurring LCOs, such as those described in Spaink, 2000, Crit. Rev. Plant Sci. 54: 257-288 and D'Haeze, et al., 2002, Glycobiology 12: 79R-105R. Precursor oligosaccharide molecules (COs, which as described below, are also useful as plant signal molecules) for the construction of LCOs may also be synthesized by genetically engineered organisms, e.g., as in Samain, et al., 1997, Carb. Res. 302: 35-42; Samain, et al., 1999, J. Biotechnol. 72: 33-47.

LCOs may be utilized in various forms of purity and may be used alone or in the form of a culture of LCO-producing bacteria or fungi. Methods to provide substantially pure LCOs include simply removing the microbial cells from a mixture of LCOs and the microbe, or continuing to isolate and purify the LCO molecules through LCO solvent phase separation followed by HPLC chromatography as described, for example, in U.S. Pat. No. 5,549,718. Purification can be enhanced by repeated HPLC, and the purified LCO molecules can be freeze-dried for long-term storage.

Chitooligosaccharides. The formulations described herein may comprise one or more chitooligosaccharides as plant signal molecules. Chitooligosaccharides (COs) are known in the art as β-1-4-linked N-acetyl glucosamine structures identified as chitin oligomers, also as N-acetylchitooligosaccharides. COs have unique and different side chain decorations which make them different from chitin molecules [(C₈—H₁₃NO₅)ₙ, CAS No. 1398-61-4], and chitosan molecules [(C₅H₁₁NO₄)ₙ, CAS No. 9012-76-4]. Representative literature describing the structure and production of COs is as follows: Van der Hoist et al., Current Opinion in Structural Biology 11: 608-616 (2001); Robina, et al., 2002, Tetrahedron 58: 521-530; Hanel et al., 2010, Planta 232: 787-806; Rouge, et al. Chapter 27, “The Molecular Immunology of Complex Carbohydrates” in Advances in Experimental Medicine and Biology, Springer Science; Wan et al., 2009, Plant Cell 21: 1053-1069; PCT/F100/00803 (Sep. 21, 2000); and Demont-Caulet et al., 1999, Plant Physiol. 120(1): 83-92 (1999). The COs may be synthetic or recombinant. Methods for preparation of recombinant COs are known in the art. See, e.g., Samain et al., 1997, supra; Samain, et al., 1999, supra; and Cottaz et al., 2005, Meth. Eng. 7(4): 311-317. COs are intended to include isomers, salts, and solvates thereof.

Chitinous Compounds. The formulations described herein may comprise one or more chitinous compounds as plant signal molecules. Chitins and chitosans, which are major components of the cell walls of fungi and the exoskeletons of insects and crustaceans, are also composed of GlcNAc residues. Chitinous compounds include chitin, (IUPAC: N-[5-[[3-acetylamino-4,5-dihydroxy-6-(hydroxymethyl)oxan-2yl]methoxymethyl]-2-[[5-acetylamino-4,6-dihydroxy-2-(hydroxymethyl)oxan-3-yl]methoxymethyl]-4-hydroxy-6-(hydroxymethyl)oxan-3-ys]ethanamide), chitosan, (IUPAC: 5-amino-6-[5-amino-6-[5-amino-4,6-dihydroxy-2(hydroxymethyl)oxan-3-yl]oxy-4-hydroxy-2-(hydroxymethyl)oxan-3-yl]oxy-2(hydroxymethyl)oxane-3,4-diol), and isomers, salts, and solvates thereof.

These compounds may be obtained commercially (e.g., from Sigma-Aldrich), or may be prepared from insects, crustacean shells, or fungal cell walls. Methods for the preparation of chitin and chitosan are known in the art, and have been described, for example, in U.S. Pat. No. 4,536,207 (preparation from crustacean shells), Pochanavanich et al., Lett. Appl. Microbiol. 35:17-21 (2002) (preparation from fungal cell walls), and U.S. Pat. No. 5,965,545 (preparation from crab shells and hydrolysis of commercial chitosan). Deacetylated chitins and chitosans may be obtained that range from less than 35% to greater than 90% deacetylation, and cover a broad spectrum of molecular weights, e.g., low molecular weight chitosan oligomers of less than 15 kDa and chitin oligomers of 0.5 to 2 kDa; “practical grade” chitosan with a molecular weight of about 15 kDa; and high molecular weight chitosan of up to 70 kDa. Chitin and chitosan compositions formulated for seed treatment are also commercially available. Commercial products include, for example, ELEXA® (Plant Defense Boosters, Inc.) and BEYOND™ (Agrihouse, Inc.).

Flavonoids. The formulations described herein may comprise one or more flavonoids as plant signal molecules. Flavonoids are phenolic compounds having the general structure of two aromatic rings connected by a three-carbon bridge. Flavonoids are produced by plants and have many functions, e.g., as beneficial signaling molecules, and as protection against insects, animals, fungi and bacteria. Classes of flavonoids are known in the art. See, Jain et al., 2002, J. Plant Biochem. & Biotechnol. 11: 1-10; Shaw et al., 2006, Environmental Microbiol. 11: 1867-80. Flavonoid compounds are commercially available, e.g., from Novozymes BioAg, Saskatoon, Canada; Natland International Corp., Research Triangle Park, N.C.; MP Biomedicals, Irvine, Calif.; LC Laboratories, Woburn Mass. Flavonoid compounds may be isolated from plants or seeds, e.g., as described in U.S. Pat. Nos. 5,702,752; 5,990,291; and 6,146,668. Flavonoid compounds may also be produced by genetically engineered organisms, such as yeast, as described in Ralston, et al., 2005, Plant Physiology 137: 1375-88. Flavonoid compounds are intended to include all flavonoid compounds as well as isomers, salts, and solvates thereof.

The one or more flavonoids may be a natural flavonoid (i.e., not synthetically produced), a synthetic flavonoid (e.g., a chemically synthesized flavonoid), or a combination thereof. In a particular embodiment, the formulations described herein comprise a flavanol, a flavone, an anthocyanidin, an isoflavonoid, a neoflavonoid, and combinations thereof, including all isomer, solvate, hydrate, polymorphic, crystalline, non-crystalline, and salt variations thereof.

In an embodiment, the formulations described herein may comprise one or more flavanols. In still another embodiment, the formulations described herein may comprise one or more flavanols selected from the group consisting of flavan-3-ols (e.g., catechin (C), gallocatechin (GC), catechin 3-gallate (Cg), gallocatechin 3-gallate (GCg), epicatechins (EC), epigallocatechin (EGC) epicatechin 3-gallate (ECg), epigallocatechin 3-gallate (EGCg), etc.), flavan-4-ols, flavan-3,4-diols (e.g., leucoanthocyanidin), and proanthocyanidins (e.g., includes dimers, trimer, oligomers, or polymers of flavanols). In still yet another embodiment, the formulations described herein may comprise one or more flavanols selected from the group consisting of catechin (C), gallocatechin (GC), catechin 3-gallate (Cg), gallocatechin 3-gallate (GCg), epicatechins (EC), epigallocatechin (EGC) epicatechin 3-gallate (ECg), epigallocatechin 3-gallate (EGCg), flavan-4-ol, leucoanthocyanidin, and dimers, trimers, oligomers or polymers thereof.

In another embodiment, the formulations described herein may comprise one or more flavones. In still another embodiment, the formulations described herein may comprise one or more flavones selected from the group consisting of flavones (e.g., luteolin, apigenin, tangeritin, etc.), flavonols (e.g., quercetin, quercitrin, rutin, kaempferol, kaempferitrin, astragalin, sophoraflavonoloside, myricetin, fisetin, isorhamnetin, pachypodol, rhamnazin, etc.), flavanones (e.g., hesperetin, hesperidin, naringenin, eriodictyol, homoeriodictyol, etc.), and flavanonols (e.g., dihydroquercetin, dihydrokaempferol, etc.). In still yet another embodiment, the formulations described herein may comprise one or more flavones selected from the group consisting of luteolin, apigenin, tangeritin, quercetin, quercitrin, rutin, kaempferol, kaempferitrin, astragalin, sophoraflavonoloside, myricetin, fisetin, isorhamnetin, pachypodol, rhamnazin, hesperetin, hesperidin, naringenin, eriodictyol, homoeriodictyol, dihydroquercetin, and dihydrokaempferol.

In still another embodiment, the formulations described herein may comprise one or more anthocyanidins. In yet another embodiment, the formulations described herein may comprise one or more anthocyanidins selected from the group selected from the group consisting of cyanidins, delphinidins, malvidins, pelargonidins, peonidins, and petunidins.

In another embodiment, the formulations described herein may comprise one or more isoflavonoids. In still yet another embodiment, the formulations described herein comprise one or more isoflavonoids selected from the group consisting of phytoestrogens, isoflavones (e.g., genistein, daidzein, glycitein, etc.), and isoflavanes (e.g., equol, lonchocarpane, laxiflorane, etc.). In yet another embodiment the formulations described herein may comprise one or more isoflavonoids selected from the group consisting of genistein, daidzein, glycitein, equol, lonchocarpane, and laxiflorane.

In another embodiment, the formulations described herein may comprise one or more neoflavonoids. In another embodiment, the formulations described herein may comprise one or more neoflavonoids selected from the group consisting of coutareagenins, dalbergins, neoflavenes (e.g., dalbergichromene), neoflavones (e.g., calophyllolide), and nivetins. In another embodiment, the formulations described herein may comprise one or more neoflavonoids selected from the group consisting of calophyllolide, coutareagenin, dalbergichromene, dalbergin, and nivetin.

In another embodiment, the formulations described herein may comprise one or flavonoids selected from the group consisting of catechin (C), gallocatechin (GC), catechin 3-gallate (Cg), gallocatechin 3-gallate (GCg), epicatechins (EC), epigallocatechin (EGO) epicatechin 3-gallate (ECg), epigallocatechin 3-gallate (EGCg), flavan-4-ol, leucoanthocyanidin, proanthocyanidins, luteolin, apigenin, tangeritin, quercetin, quercitrin, rutin, kaempferol, kaempferitrin, astragalin, sophoraflavonoloside, myricetin, fisetin, isorhamnetin, pachypodol, rhamnazin, hesperetin, hesperidin, naringenin, eriodictyol, homoeriodictyol, dihydroquercetin, dihydrokaempferol, cyanidins, delphinidins, malvidins, pelargonidins, peonidins, petunidins, genistein, daidzein, glycitein, equol, lonchocarpane, laxiflorane, calophyllolide, dalbergichromene, coutareagenin, dalbergin, and nivetin. In another embodiment, the formulations described herein may comprise one or more flavonoids selected from the group consisting of hesperetin, hesperidin, naringenin, genistein, and daidzein. In a particular embodiment, the formulation described herein may comprise the flavonoid hesperetin. In another particular embodiment, the formulation described herein may comprise the flavonoid hesperidin. In another particular embodiment, the formulation described herein may comprise the flavonoid naringenin. In another particular embodiment, the formulation described herein may comprise the flavonoid genistein. In another particular embodiment, the formulation described herein may comprise the flavonoid daidzein.

Non-Flavonoid Nod-Gene Inducers. The formulations described herein may comprise one or more non-flavonoid nod-gene inducers as plant signal molecules. Examples of non-flavonoid nod-gene inducers include, but are not limited to, jasmonic acid (JA, [1R-[1α,2β(Z)]]-3-oxo-2-(pentenyl)cyclopentaneacetic acid) and its derivatives, linoleic acid ((Z,Z)-9,12-octadecadienoic acid) and its derivatives, and linolenic acid ((Z,Z,Z)-9,12,15-octadecatrienoic acid) and its derivatives. Non-flavonoid nod-gene inducers are intended to include not only the non-flavonoid nod-gene inducers described herein, but isomers, salts, and solvates thereof.

Jasmonic acid and its methyl ester, methyl jasmonate (MeJA), collectively known as jasmonates, are octadecanoid-based compounds that occur naturally in plants. Jasmonic acid is produced by the roots of wheat seedlings, and by fungal microorganisms such as Botryodiplodia theobromae and Gibberella fujikuroi, yeast (Saccharomyces cerevisiae), and pathogenic and non-pathogenic strains of Escherichia coli. Linoleic acid and linolenic acid are produced in the course of the biosynthesis of jasmonic acid. Jasmonates, linoleic acid and linoleic acid (and their derivatives) are reported to be inducers of nod gene expression or LCO production by rhizobacteria.

Derivatives of linoleic acid, linolenic acid, and jasmonic acid that may be useful in the formulations of the present invention include esters, amides, glycosides and salts. Representative esters are compounds in which the carboxyl group of linoleic acid, linolenic acid, or jasmonic acid has been replaced with a —COR group, wherein R is an alkyl group, such as a C₁-C₈ unbranched or branched alkyl group, e.g., a methyl, ethyl or propyl group; an alkenyl group, such as a C₂-C₈ unbranched or branched alkenyl group; an alkynyl group, such as a C₂-C₈ unbranched or branched alkynyl group; an aryl group having, for example, 6 to 10 carbon atoms; or a heteroaryl group having, for example, 4 to 9 carbon atoms, wherein the heteroatoms in the heteroaryl group can be, for example, N, O, P, or S. Representative amides are compounds in which the carboxyl group of linoleic acid, linolenic acid, or jasmonic acid has been replaced with a —COR group, where R is an NR²R³ group, in which R² and R³ are independently: hydrogen; an alkyl group, such as a C₁-C₈ unbranched or branched alkyl group, e.g., a methyl, ethyl or propyl group; an alkenyl group, such as a C₂-C₈ unbranched or branched alkenyl group; an alkynyl group, such as a C₂-C₈ unbranched or branched alkynyl group; an aryl group having, for example, 6 to 10 carbon atoms; or a heteroaryl group having, for example, 4 to 9 carbon atoms, wherein the heteroatoms in the heteroaryl group can be, for example, N, O, P, or S. Esters may be prepared by known methods, such as acid-catalyzed nucleophilic substitution, wherein the carboxylic acid is reacted with an alcohol in the presence of a catalytic amount of a mineral acid. Amides may also be prepared by known methods, such as by reacting the carboxylic acid with the appropriate amine in the presence of a coupling agent such as dicyclohexyl carbodiimide (DCC), under neutral conditions. Suitable salts of linoleic acid, linolenic acid, and jasmonic acid include e.g., base addition salts. The bases that may be used as reagents to prepare metabolically acceptable base salts of these compounds include those derived from cations such as alkali metal cations (e.g., potassium and sodium) and alkaline earth metal cations (e.g., calcium and magnesium). These salts may be readily prepared by mixing together a solution of linoleic acid, linolenic acid, or jasmonic acid with a solution of the base. The salt may be precipitated from solution and may be collected by filtration or may be recovered by other means such as by evaporation of the solvent.

Karrikins. The formulations described herein may comprise one or more karrikins as plant signal molecules. Karrikins are vinylogous 4H-pyrones e.g., 2H-furo[2,3-c]pyran-2-ones including derivatives and analogues thereof. It is intended that the karrikins include isomers, salts, and solvates thereof. Examples of biologically acceptable salts of these compounds may include acid addition salts formed with biologically acceptable acids, examples of which include hydrochloride, hydrobromide, sulphate or bisulphate, phosphate or hydrogen phosphate, acetate, benzoate, succinate, fumarate, maleate, lactate, citrate, tartrate, gluconate; methanesulphonate, benzenesulphonate, and p-toluenesulphonic acid. Additional biologically acceptable metal salts may include alkali metal salts, with bases, examples of which include the sodium and potassium salts. Examples of compounds embraced by the structure and which may be suitable for use in the present invention include the following: 3-methyl-2H-furo[2,3-c]pyran-2-one (where R₁═CH₃, R₂, R₃, R₄═H), 2H-furo[2,3-c]pyran-2-one (where R₁, R₂, R₃, R4═H), 7-methyl-2H-furo[2,3-c]pyran-2-one (where R₁, R₂, R₄═H, R₃═CH₃), 5-methyl-2H-furo[2,3-c]pyran-2-one (where R₁, R₂, R₃═H, R₄═CH₃), 3,7-dimethyl-2H-furo[2,3-c]pyran-2-one (where R₁, R₃═CH₃, R₂, R₄═H), 3,5-dimethyl-2H-furo[2,3-c]pyran-2-one (where R₁, R₄═CH₃, R₂, R₃═H), 3,5,7-trimethyl-2H-furo[2,3-c]pyran-2-one (where R₁, R₃, R₄═CH₃, R₂═H), 5-methoxymethyl-3-methyl-2H-furo[2,3-c]pyran-2-one (where R₁═CH₃, R₂, R₃═H, R₄═CH₂OCH₃), 4-bromo-3,7-dimethyl-2H-furo[2,3-c]pyran-2-one (where R₁, R₃═CH₃, R₂═Br, R₄═H), 3-methylfuro[2,3-c]pyridin-2(3H)-one (where Z═NH, R₁═CH₃, R₂, R₃, R₄═H), 3,6-dimethylfuro[2,3-c]pyridin-2(6H)-one (where Z═N—CH₃, R₁═CH₃, R₂, R₃, R₄═H). See U.S. Pat. No. 7,576,213. These molecules are also known as karrikins. See, Halford, “Smoke Signals,” in Chem. Eng. News (Apr. 12, 2010), at pages 37-38 (reporting that karrikins or butenolides which are contained in smoke act as growth stimulants and spur seed germination after a forest fire, and can invigorate seeds such as corn, tomatoes, lettuce, and onions that had been stored). These molecules are the subject of U.S. Pat. No. 7,576,213.

Nutrients:

The formulations described herein may comprise or further comprise one or more beneficial nutrients. Non-limiting examples of nutrients for use in the formulations described herein include vitamins, (e.g., vitamin A, vitamin B complex, i.e., vitamin B₁, vitamin B₂, vitamin B₃, vitamin B₅, vitamin B₆, vitamin B₇, vitamin B₈, vitamin B₉, vitamin B₁₂, choline) vitamin C, vitamin D, vitamin E, vitamin K, carotenoids (α-carotene, β-carotene, cryptoxanthin, lutein, lycopene, zeaxanthin, etc.), macrominerals (e.g., phosphorus, calcium, magnesium, potassium, sodium, iron, etc.), trace minerals (e.g., boron, cobalt, chloride, chromium, copper, fluoride, iodine, iron, manganese, molybdenum, selenium, zinc, etc.), organic acids (e.g., acetic acid, citric acid, lactic acid, malic acid, taurine, etc.), and combinations thereof. In a particular embodiment, the formulations may comprise phosphorus, boron, chlorine, copper, iron, manganese, molybdenum, zinc, or combinations thereof.

In certain embodiments, where the formulations described herein may comprise phosphorus, it is envisioned that any suitable source of phosphorus may be provided. In one embodiment, the phosphorus from the provided source may be readily solubilized. In another embodiment, phosphorus from the provided source is phosphorus capable of solubilization by one or more microorganisms (e.g., Penicillium bitaiae, etc.).

In one embodiment, the phosphorus may be derived from a rock phosphate source. In another embodiment the phosphorus may be derived from fertilizers comprising one or more phosphorus sources. Commercially available manufactured phosphate fertilizers are of many types. Some common ones are those containing rock phosphate, monoammonium phosphate, diammonium phosphate, monocalcium phosphate, super phosphate, triple super phosphate, and/or ammonium polyphosphate. All of these fertilizers are produced by chemical processing of insoluble natural rock phosphates in large scale fertilizer-manufacturing facilities and the product is expensive. By means of the present invention it is possible to reduce the amount of these fertilizers applied to the soil while still maintaining the same amount of phosphorus uptake from the soil, or the amount of phosphorus accessible by the plant.

In still another embodiment, the phosphorus may be derived from an organic phosphorus source. In a further particular embodiment, the source of phosphorus may include an organic fertilizer. An organic fertilizer refers to a soil amendment derived from natural sources that guarantees, at least, the minimum percentages of nitrogen, phosphate, and potash. Non-limiting examples of organic fertilizers include plant and animal by-products, rock powders, seaweed, inoculants, and conditioners. These are often available at garden centers and through horticultural supply companies. In particular the organic source of phosphorus is from bone meal, meat meal, animal manure, compost, sewage sludge, guano, or combinations thereof.

In still another embodiment, the phosphorus may be derived from a combination of phosphorus sources including, but not limited to, rock phosphate, fertilizers comprising one or more phosphorus sources (e.g., monoammonium phosphate, diammonium phosphate, monocalcium phosphate, super phosphate, triple super phosphate, ammonium polyphosphate, etc.), and combinations thereof.

Biostimulants:

The formulations described herein may comprise or further comprise one or more beneficial biostimulants. Biostimulants may enhance metabolic or physiological processes such as respiration, photosynthesis, nucleic acid uptake, ion uptake, nutrient delivery, or a combination thereof. Non-limiting examples of biostimulants include seaweed extracts (e.g., ascophyllum nodosum), humic acids (e.g., potassium humate), fulvic acids, myo-inositol, glycine, and combinations thereof. In another embodiment, the formulations comprise seaweed extracts, humic acids, fulvic acids, myo-inositol, glycine, and combinations thereof.

Polymers:

The formulations described herein may comprise or further comprise one or more polymers. Non-limiting uses of polymers in the agricultural industry include agrochemical delivery, heavy metal removal, water retention and/or water delivery, and combinations thereof. Pouci et al., 2008, Am. J. Agri. & Biol. Sci. 3(1): 299-314. In one embodiment, the one or more polymers are selected from the group consisting of a natural polymer (e.g., agar, starch, alginate, pectin, cellulose, etc.), a synthetic polymer, and a biodegradable polymer (e.g., polycaprolactone, polylactide, poly (vinyl alcohol), etc.).

For a non-limiting list of polymers useful for the formulations described herein, see Pouci et al., 2008, supra. In one embodiment, the formulations described herein comprise cellulose, cellulose derivatives, methylcellulose, methylcellulose derivatives, starch, agar, alginate, pectin, polyvinylpyrrolidone, and combinations thereof.

Wetting Agents:

The formulations described herein may further comprise one or more wetting agents. Wetting agents are commonly used on soils, particularly hydrophobic soils, to improve the infiltration and/or penetration of water into a soil. The wetting agent may be an adjuvant, oil, surfactant, buffer, acidifier, or combination thereof. In an embodiment, the wetting agent is a surfactant. In another embodiment, the wetting agent is one or more nonionic surfactants, one or more anionic surfactants, or a combination thereof. In yet another embodiment, the wetting agent is one or more nonionic surfactants.

Surfactants:

The formulations described herein may comprise or further comprise one or more surfactants. Surfactants suitable for the formulations described herein may be non-ionic surfactants (e.g., semi-polar and/or anionic and/or cationic and/or zwitterionic). The surfactants can wet and emulsify soil(s) and/or dirt(s). It is envisioned that the surfactants used have low toxicity for any microorganisms contained within the formulation. It is further envisioned that the surfactants used have a low phytotoxicity (i.e., the degree of toxicity a substance or combination of substances has on a plant). A single surfactant or a blend of several surfactants can be used.

Anionic surfactants: Anionic surfactants or mixtures of anionic and nonionic surfactants may also be used in the formulations. Anionic surfactants are surfactants having a hydrophilic moiety in an anionic or negatively charged state in aqueous solution. The formulations described herein may comprise one or more anionic surfactants. The anionic surfactant(s) may be either water soluble anionic surfactants, water insoluble anionic surfactants, or a combination of water soluble anionic surfactants and water insoluble anionic surfactants. Non-limiting examples of anionic surfactants include sulfonic acids, sulfuric acid esters, carboxylic acids, and salts thereof. Non-limiting examples of water soluble anionic surfactants include alkyl sulfates, alkyl ether sulfates, alkyl amido ether sulfates, alkyl aryl polyether sulfates, alkyl aryl sulfates, alkyl aryl sulfonates, monoglyceride sulfates, alkyl sulfonates, alkyl amide sulfonates, alkyl aryl sulfonates, benzene sulfonates, toluene sulfonates, xylene sulfonates, cumene sulfonates, alkyl benzene sulfonates, alkyl diphenyloxide sulfonate, alpha-olefin sulfonates, alkyl naphthalene sulfonates, paraffin sulfonates, lignin sulfonates, alkyl sulfosuccinates, ethoxylated sulfosuccinates, alkyl ether sulfosuccinates, alkylamide sulfosuccinates, alkyl sulfosuccinamate, alkyl sulfoacetates, alkyl phosphates, phosphate ester, alkyl ether phosphates, acyl sarconsinates, acyl isethionates, N-acyl taurates, N-acyl-N-alkyltaurates, alkyl carboxylates, or a combination thereof.

Nonionic surfactants: Nonionic surfactants are surfactants having no net electrical charge when dissolved or dispersed in an aqueous medium. In at least one embodiment, one or more nonionic surfactants are used as they provide the desired wetting and emulsification actions and do not significantly inhibit spore stability and activity. The nonionic surfactant(s) may be either water soluble nonionic surfactants, water insoluble nonionic surfactants, or a combination of water soluble nonionic surfactants and water insoluble nonionic surfactants.

Water insoluble nonionic surfactants: Non-limiting examples of water insoluble nonionic surfactants include alkyl and aryl: glycerol ethers, glycol ethers, ethanolamides, sulfoanylamides, alcohols, amides, alcohol ethoxylates, glycerol esters, glycol esters, ethoxylates of glycerol ester and glycol esters, sugar-based alkyl polyglycosides, polyoxyethylenated fatty acids, alkanolamine condensates, alkanolamides, tertiary acetylenic glycols, polyoxyethylenated mercaptans, carboxylic acid esters, polyoxyethylenated polyoxyproylene glycols, sorbitan fatty esters, or combinations thereof. Also included are EO/PO block copolymers (EO is ethylene oxide, PO is propylene oxide), EO polymers and copolymers, polyamines, and polyvinylpynolidones.

Water soluble nonionic surfactants: Non-limiting examples of water soluble nonionic surfactants include sorbitan fatty acid alcohol ethoxylates and sorbitan fatty acid ester ethoxylates.

Combination of nonionic surfactants: In one embodiment, the formulations described herein comprise at least one or more nonionic surfactants. In another embodiment, the formulations comprise at least one water insoluble nonionic surfactant and at least one water soluble nonionic surfactant. In still another embodiment, the formulations comprise a combination of nonionic surfactants having hydrocarbon chains of substantially the same length.

Other Surfactants: In another embodiment, the formulations described herein may also comprise organosilicone surfactants, silicone-based antifoams used as surfactants in silicone-based and mineral-oil based antifoams. In yet another embodiment, the formulations described herein may also comprise alkali metal salts of fatty acids (e.g., water soluble alkali metal salts of fatty acids and/or water insoluble alkali metal salts of fatty acids).

Anti-Freezing Agents:

The formulations described herein may comprise or further comprise one or more anti-freezing agents. Non-limiting examples of anti-freezing agents include ethylene glycol, propylene glycol, urea, glycerin, anti-freeze proteins, and combinations thereof.

Minerals:

The formulations described herein may comprise or further comprise one or more minerals. Non-limiting examples of minerals include kaolin, silica, titanium (IV) oxide, rutile, anatase, aluminum oxides, aluminum hydroxides, iron oxide, iron sulfide, magnetite, pyrite, hematite, ferrite, gregite, calcium carbonate, calcite, aragonite, quartz, zircon, olivine, orthopyroxene, tourmaline, kyanite, albite, anorthite, clinopyroxene, orthoclase, gypsum, andalusite, talc, fluorite, apatite, orthoclase, topaz, corundum, diamond, tin, tin oxides, antimony, antimony oxides, beryllium, cobalt, copper, feldspar, gallium, indium, lead, lithium, manganese, mica, molybdenum, nickel, perlite, platinum group metals, phosphorus and phosphate rock, potash, rare earth elements, tantalum, tungsten, vanadium, zeolites, zinc and zinc oxide, and indium tin oxide.

Microbially Stabilizing Compounds:

The formulations described herein may comprise or further comprise one or more microbially stabilizing compounds. Non-limiting examples of microbially stabilizing compounds include yeast extract, calcium caseinate, milk, urea, hematinic agents, beef extract, ammonia, amino acids, ammonium salts, ferric salts, ferrous salts, gluconolactone, glutathione, lecithin, polysorbates, albumin, peptones, and combinations thereof.

### Preparation of Formulations

The present invention also relates to methods of formulating one or more (e.g., several) agriculturally beneficial agents, comprising reacting the one or more (e.g., several) agriculturally beneficial agents with (a) a composition comprising a xyloglucan endotransglycosylase, a polymeric xyloglucan, and a functionalized xyloglucan oligomer comprising a chemical group; (b) a composition comprising a xyloglucan endotransglycosylase, a polymeric xyloglucan functionalized with a chemical group, and a functionalized xyloglucan oligomer comprising a chemical group; (c) a composition comprising a xyloglucan endotransglycosylase, a polymeric xyloglucan functionalized with a chemical group, and a xyloglucan oligomer; (d) a composition comprising a xyloglucan endotransglycosylase, a polymeric xyloglucan, and a xyloglucan oligomer; (e) a composition comprising a xyloglucan endotransglycosylase and a polymeric xyloglucan functionalized with a chemical group; (f) a composition comprising a xyloglucan endotransglycosylase and a polymeric xyloglucan; (g) a composition comprising a xyloglucan endotransglycosylase and a functionalized xyloglucan oligomer comprising a chemical group; (h) a composition comprising a xyloglucan endotransglycosylase and a xyloglucan oligomer, or (i) a composition of (a), (b), (c), (d), (e), (f), (g), or (h) without a xyloglucan endotransglycosylase.

The formulations of the present invention comprise one or more agriculturally beneficial agents formulated with a polymeric xyloglucan or a polymeric xyloglucan functionalized with a chemical group as a carrier for imparting an agricultural benefit to a seed, a plant, a plant part, a soil, or a combination thereof. The one or more agriculturally beneficial agents can be linked to, coated by, embedded in, or encapsulated by the polymeric xyloglucan or the polymeric xyloglucan functionalized with a chemical group as a carrier. The formulations can be in any form known in the art including, but not limited to, an aerosol, emulsifiable concentrate, wettable powder, soluble concentrate, soluble powder, suspension concentrate, spray concentrate, capsule suspension, water dispersible granule, granules, dusts, microgranule, gel, hydrogel, or seed treatment formulation. Liquid formulations may be suitable for foliar application to a plant or plant part.

An agriculturally beneficial agent can be formulated with a polymeric xyloglucan as a carrier by mixing the agent with the polymeric xyloglucan.

An agriculturally beneficial agent can also be formulated with a polymeric xyloglucan as a carrier by reacting the agent with the polymeric xyloglucan and a xyloglucan endotransglycosylase.

An agriculturally beneficial agent can also be formulated with a polymeric xyloglucan as a carrier by reacting the agent with the polymeric xyloglucan, a xyloglucan oligomer, and a xyloglucan endotransglycosylase.

An agriculturally beneficial agent can also be formulated with a polymeric xyloglucan functionalized with a chemical group as a carrier by mixing the agent with the polymeric xyloglucan functionalized with a chemical group.

An agriculturally beneficial agent can also be formulated with a polymeric xyloglucan functionalized with a chemical group as a carrier by mixing the agent with the polymeric xyloglucan functionalized with a chemical group and a xyloglucan endotransglycosylase.

An agriculturally beneficial agent can also be formulated with a polymeric xyloglucan as a carrier by reacting the agent with the polymeric xyloglucan, a xyloglucan oligomer functionalized with a chemical group, and a xyloglucan endotransglycosylase.

An agriculturally beneficial agent can also be formulated with a polymeric xyloglucan functionalized with a chemical group as a carrier by reacting the agent with the polymeric xyloglucan functionalized with a chemical group, a xyloglucan oligomer, and a xyloglucan endotransglycosylase.

An agriculturally beneficial agent can also be formulated with a polymeric xyloglucan functionalized with a chemical group as a carrier by reacting the agent with the polymeric xyloglucan functionalized with a chemical group, a xyloglucan oligomer functionalized with a chemical group, and a xyloglucan endotransglycosylase.

In each of the aspects above involving a xyloglucan oligomer functionalized with a chemical group, an agriculturally beneficial agent can be covalently bound to the xyloglucan oligomer functionalized with a chemical group via the chemical group prior to formulation. In each of the aspects above involving a polymeric xyloglucan functionalized with a chemical group, an agriculturally beneficial agent can be covalently bound to the polymeric xyloglucan functionalized with a chemical group via the chemical group prior to formulation. In each of the aspects above involving a xyloglucan oligomer functionalized with a chemical group and a polymeric xyloglucan functionalized with a chemical group, an agriculturally beneficial agent can be covalently bound to the xyloglucan oligomer functionalized with a chemical group via the chemical group and the polymeric xyloglucan functionalized with a chemical group via the chemical group prior to formulation.

In another aspect, the linking of the one or more agriculturally beneficial agents to the polymeric xyloglucan is via a covalent bond between the one or more agriculturally beneficial agents and the polymeric xyloglucan.

In each of the aspects above involving a xyloglucan oligomer functionalized with a chemical group, an agriculturally beneficial agent can be electrostatically associated with the xyloglucan oligomer functionalized with a chemical group via the chemical group. In each of the aspects above involving a polymeric xyloglucan functionalized with a chemical group, an agriculturally beneficial agent can be electrostatically associated with the polymeric xyloglucan functionalized with a chemical group via the chemical group. In each of the aspects above involving a xyloglucan oligomer functionalized with a chemical group and a polymeric xyloglucan functionalized with a chemical group, an agriculturally beneficial agent can be electrostatically associated with the xyloglucan oligomer functionalized with a chemical group via the chemical group and the polymeric xyloglucan functionalized with a chemical group via the chemical group.

In each of the aspects above involving a xyloglucan oligomer functionalized with a chemical group, an agriculturally beneficial agent can be hydrophobically associated with (e.g., forms Vander Waals bonds to or is entropically linked with via exclusion of polar or aqueous solvent) the xyloglucan oligomer functionalized with a chemical group via the chemical group. In each of the aspects above involving a polymeric xyloglucan functionalized with a chemical group, an agriculturally beneficial agent can be hydrophobically associated with the polymeric xyloglucan functionalized with a chemical group via the chemical group. In each of the aspects above involving a xyloglucan oligomer functionalized with a chemical group and a polymeric xyloglucan functionalized with a chemical group, an agriculturally beneficial agent can be hydrophobically associated with the xyloglucan oligomer functionalized with a chemical group via the chemical group and the xyloglucan oligomer functionalized with a chemical group via the chemical group.

In each of the aspect above, the linking of the one or more agriculturally beneficial agents to the polymeric xyloglucan can be via a combination of covalent and hydrophobic interactions with the chemical group of the xyloglucan oligomer functionalized with the chemical group. In each of the aspect above, the linking of the one or more agriculturally beneficial agents to the polymeric xyloglucan can be via a combination of hydrophobic and electrostatic interactions with the chemical group of the polymeric xyloglucan functionalized with the chemical group. In each of the aspect above, the linking of the one or more agriculturally beneficial agents to the polymeric xyloglucan can be via a combination of covalent and hydrophobic interactions with the chemical group of the xyloglucan oligomer functionalized with the chemical group and a combination of covalent and hydrophobic interactions with the chemical group of the polymeric xyloglucan functionalized with the chemical group. In each of the aspects above, the chemical group can have additional affinity or specificity for plant tissue.

In each of the aspects above, the linking of the one or more agriculturally beneficial agents to the polymeric xyloglucan can be via a combination of covalent and electrostatic interactions with the chemical group of the xyloglucan oligomer functionalized with the chemical group. In each of the aspects above, the linking of the one or more agriculturally beneficial agents to the polymeric xyloglucan can be via a combination of covalent and electrostatic interactions with the chemical group of the polymeric xyloglucan functionalized with the chemical group. In each of the aspects above, the linking of the one or more agriculturally beneficial agents to the polymeric xyloglucan can be via a combination of covalent and electrostatic interactions with the chemical group of the xyloglucan oligomer functionalized with the chemical group and a combination of hydrophobic and electrostatic interactions with the chemical group of the polymeric xyloglucan functionalized with the chemical group. In each of the aspects above, the chemical group can have additional affinity or specificity for plant tissue.

In each of the aspects above, the linking of the one or more agriculturally beneficial agents to the polymeric xyloglucan can be via a combination of hydrophobic and electrostatic interactions with the chemical group of the xyloglucan oligomer functionalized with the chemical group. In each of the aspects above, the linking of the one or more agriculturally beneficial agents to the polymeric xyloglucan can be via a combination of hydrophobic and electrostatic interactions with the chemical group of the polymeric xyloglucan functionalized with the chemical group. In each of the aspects above, the linking of the one or more agriculturally beneficial agents to the polymeric xyloglucan can be via a combination of hydrophobic and electrostatic interactions with the chemical group of the xyloglucan oligomer functionalized with the chemical group and a combination of hydrophobic and electrostatic interactions with the chemical group of the polymeric xyloglucan functionalized with the chemical group. In each of the aspects above, the chemical group can have additional affinity or specificity for plant tissue.

In each of the aspects above, the linking of the one or more agriculturally beneficial agents to the polymeric xyloglucan can be via a combination of covalent, hydrophobic, and electrostatic interactions with the chemical group of the xyloglucan oligomer functionalized with the chemical group. In each of the aspects above, the linking of the one or more agriculturally beneficial agents to the polymeric xyloglucan can be via a combination of covalent, hydrophobic, and electrostatic interactions with the chemical group of the polymeric xyloglucan functionalized with the chemical group. In each of the aspects above, the linking of the one or more agriculturally beneficial agents to the polymeric xyloglucan can be via a combination of covalent, hydrophobic, and electrostatic interactions with the chemical group of the xyloglucan oligomer functionalized with the chemical group and a combination of covalent, hydrophobic, and electrostatic interactions with the chemical group of the polymeric xyloglucan functionalized with the chemical group. In each of the aspects above, the chemical group can have additional affinity or specificity for plant tissue.

An agricultural benefit can be generated by treating a seed, a plant, a plant part, a soil, or a combination thereof, with a formulation of the present invention under conditions leading to association between the composition and the seed, plant, plant part, a soil, or combination thereof. Application of a formulation to a target can be accomplished using any delivery method known in the art including, but not limited to, dusting, fumigation, granule application, injection, misting, seed treatment, spraying, dipping, or coating. In one aspect, the aqueous medium is provided by or generated by the plant, plant tissues, soil or environment, as any or all of these may contain water. In such case, the application may be performed dry.

The methods are exemplified by, but are not limited to, association of an easily tracked agent such as fluorescein-labeled agent with a seed, a plant, a plant part, a soil, or a combination thereof, illustrating direct binding of the one or more agriculturally beneficial agents to the seed, plant, plant part, soil, or combination thereof. It is understood that a fluorescein-leaf or fluorescein-seed interaction is representative of a potential agriculturally beneficial compound, and that leaves and seeds are representative of the plant-specific tissues that can be bound, but are not limiting examples in the present invention. In the methods of the present invention, plant leaves can be incubated in a pH controlled solution, e.g., buffered solution (e.g., sodium citrate) from pH 3 to pH 9, e.g., from pH 4 to pH 8 or from pH 5 to pH 7, at concentrations from about 1 g/L to about 10 kg/L, e.g., about 10 g/L to about 1 kg/L or about 40 g/L to about 100 g/L containing xyloglucan endotransglycosylase and fluorescein isothiocyanate functionalized xyloglucan or polymeric xyloglucan with fluorescein isothiocyanate functionalized xyloglucan oligomer. The xyloglucan endotransglycosylase can be present at about 0.1 nM to about 1 mM, e.g., about 10 nM to about 100 μM or about 0.5 μM to about 5 μM. In one aspect, the xyloglucan endotransglycosylase is present at a concentration of 320 μg to about 32 mg of enzyme per g of the plant leaf, e.g., about 160 μg to about 4 mg of enzyme per g of the plant leaf. When present, the functionalized xyloglucan oligomer can be present with polymeric xyloglucan at about 50:1 molar ratio to about 0.5:1, e.g., about 10:1 to about 1:1 or about 5:1 to about 1:1. The polymeric xyloglucan can be present at about 1 mg per g of the plant leaf to about 1 g per g of the plant leaf, e.g., about 10 mg to about 100 mg per g of the plant leaf or about 20 mg to about 50 mg per g of the plant leaf. The incubation can last for a sufficient period of time to effect the desired extent of association, e.g., about instantaneously to about 72 hours, about 15 minutes to about 48 hours, about 30 minutes to about 24 hours, or about 1 to about 3 hours. In one preferred aspect, the plant part is dipped into or sprayed with the formulation.

In another aspect of the invention, the agriculturally beneficial agent is not covalently bound to the xyloglucan. Xyloglucan can be made to tightly, but non-covalently, associate with various materials via xyloglucan endotransglycosylase activity. This aspect of the invention is of great utility for the formation of physical barriers to protect agricultural crops from, for example, UV damage, insect pests, fungal infection, and other sources of harm, and is of great utility in the delivery of biological pesticides, for example, fungicidal bacteria and spores, wherein the agriculturally beneficial agent has high molecular weight or is physically large. For example, a material such as TiO₂ or kaolin is suspended in a pH controlled aqueous medium under conditions described above, sufficient to effect a xyloglucan-TiO₂ or xyloglucan-kaolin association. The slurry is then applied to plant material or soil. In one aspect, the plant or plant part is dipped in the slurry. In another aspect, the slurry is dried before application.

In another aspect of the invention, the polymeric xyloglucan is functionalized prior to contacting the plant or the soil. The polymeric xyloglucan can be incubated in a pH controlled solution with xyloglucan endotransglycosylase and functionalized xyloglucan oligomers, yielding functionalized polymeric xyloglucan. The functionalized polymeric xyloglucan can then be separated from the functionalized xyloglucan oligomers by any method known in the art, but as exemplified by ethanol precipitation. For example, the reaction mixture can be incubated in 80% (v/v) ethanol for about 1 minute to about 24 hours, e.g., 30 minutes to 20 hours or 12 hours to 15 hours, centrifuged for an appropriate length of time at an appropriate velocity to pellet the precipitated, functionalized polymeric xyloglucan (e.g., 30 minutes at approximately 2000×g), and the supernatants decanted. The functionalized polymeric xyloglucan is then optionally dried. In this aspect, the functionalized polymeric xyloglucan is then incubated with xyloglucan endotransglycosylase and the plant or soil.

In another aspect, an aqueous medium for the formulation is provided by the environment, by the plant or by the soil. In this aspect, a dry powder containing the components of the formulation is applied to the plant or the soil, and water is present on or in the plant, is present in the soil, or can be supplied either by irrigation or by rainfall.

In another aspect, the concentration of polymeric xyloglucan as a carrier is high, such that the xyloglucan forms a hydrogel of rheology appropriate to the application process. In this aspect, the gel may be used to encapsulate or enmesh the agriculturally beneficial material. In this aspect, the xyloglucan may be additionally functionalized with a chemical functional group as appropriate.

### Sources of Xyloglucan Endotransglycosylases

Any xyloglucan endotransglycosylase that possesses suitable enzyme activity at a pH and temperature appropriate for the methods of the present invention may be used. It is preferable that the xyloglucan endotransglycosylase is active over a broad pH and temperature range. In an embodiment, the xyloglucan endotransglycosylase has a pH optimum in the range of about 3 to about 10. In another embodiment, the xyloglucan endotransglycosylase has a pH optimum in the range of about 4.5 to about 8.5. In another embodiment, the xyloglucan endotransglycosylase has a cold denaturation temperature less than or equal to about 5° C. or a melting temperature of about 100° C. or higher. In another embodiment, the xyloglucan endotransglycosylase has a cold denaturation temperature of less than or equal to 20° C. or a melting temperature greater than or equal to about 75° C.

The source of the xyloglucan endotransglycosylase used is not critical in the present invention. Accordingly, the xyloglucan endotransglycosylase may be obtained from any source such as a plant, microorganism, or animal.

In one embodiment, the xyloglucan endotransglycosylase is obtained from a plant source. Xyloglucan endotransglycosylase can be obtained from cotyledons of the family Fabaceae (synonyms: Leguminosae and Papilionaceae), preferably genus Phaseolus, in particular, Phaseolus aureus. Preferred monocotyledons are non-graminaceous monocotyledons and liliaceous monocotyledons. Xyloglucan endotransglycosylase can also be extracted from moss and liverwort, as described in Fry et al., 1992, Biochem. J. 282: 821-828. For example, the xyloglucan endotransglycosylase may be obtained from cotyledons, i.e., a dicotyledon or a monocotyledon, in particular a dicotyledon selected from the group consisting of azuki beans, canola, cauliflowers, cotton, poplar or hybrid aspen, potatoes, rapes, soy beans, sunflowers, thalecress, tobacco, and tomatoes, or a monocotyledon selected from the group consisting of wheat, rice, corn, and sugar cane. See, for example, WO 2003/033813 and WO 97/23683.

In another embodiment, the xyloglucan endotransglycosylase is obtained from Arabidopsis thaliana (GENESEQP:AOE11231, GENESEQP:AOE93420, GENESEQP: BAL03414, GENESEQP:BAL03622, or GENESEQP:AWK95154); Carica papaya (GENESEQP:AZR75725); Cucumis sativus (GENESEQP:AZV66490); Daucus carota (GENESEQP:AZV66139); Festuca pratensis (GENESEQP:AZR80321); Glycine max (GENESEQP:AWK95154 or GENESEQP:AYF92062); Hordeum vulgare (GENESEQP:AZR85056, GENESEQP:AQY12558, GENESEQP:AQY12559, or GENESEQP:AWK95180); Lycopersicon esculentum (GENESEQP:ATZ45232); Medicago truncatula (GENESEQP:ATZ48025); Otyza sativa (GENESEQP:ATZ42485, GENESEQP:ATZ57524, or GENESEQP:AZR76430); Populus tremula (GENESEQP:AWK95036); Sagittaria pygmaea (GENESEQP:AZV66468); Sorghum bicolor (GENESEQP:BA079623 or GENESEQP:BA079007); Vigna angularis (GENESEQP:ATZ61320); or Zea mays (GENESEQP:AWK94916).

In another embodiment, the xyloglucan endotransglycosylase is a xyloglucan endotransglucosylase/hydrolase (XTH) with both hydrolytic and transglycosylating activities. In a preferred embodiment, the ratio of transglycosylation to hydrolytic rates is at least 10⁻² to 10⁷, e.g., 10⁻¹ to 10⁶ or 10 to 1000.

### Production of Xyloglucan Endotransglycosylases

Xyloglucan endotransglycosylase may be extracted from plants. Suitable methods for extracting xyloglucan endotransglycosylase from plants are described Fry et al., 1992, Biochem. J. 282: 821-828; Sulova et al., 1998, Biochem. J. 330: 1475-1480; Sulova et al., 1995, Anal. Biochem. 229: 80-85; WO 95/13384; WO 97/23683; or EP 562 836.

Xyloglucan endotransglycosylase may also be produced by cultivation of a transformed host organism containing the appropriate genetic information from a plant, microorganism, or animal. Transformants can be prepared and cultivated by methods known in the art.

Techniques used to isolate or clone a gene are known in the art and include isolation from genomic DNA or cDNA, or a combination thereof. The cloning of the gene from genomic DNA can be effected, e.g., by using the polymerase chain reaction (PCR) or antibody screening of expression libraries to detect cloned DNA fragments with shared structural features. See, e.g., Innis et al., 1990, PCR: A Guide to Methods and Application, Academic Press, New York. Other nucleic acid amplification procedures such as ligase chain reaction (LCR), ligation activated transcription (LAT) and polynucleotide-based amplification (NASBA) may be used.

A nucleic acid construct can be constructed to comprise a gene encoding a xyloglucan endotransglycosylase operably linked to one or more control sequences that direct the expression of the coding sequence in a suitable host cell under conditions compatible with the control sequences. The gene may be manipulated in a variety of ways to provide for expression of the xyloglucan endotransglycosylase. Manipulation of the gene prior to its insertion into a vector may be desirable or necessary depending on the expression vector. Techniques for modifying polynucleotides utilizing recombinant DNA methods are well known in the art.

The control sequence may be a promoter, a polynucleotide that is recognized by a host cell for expression of a polynucleotide encoding a xyloglucan endotransglycosylase. The promoter contains transcriptional control sequences that mediate the expression of the xyloglucan endotransglycosylase. The promoter may be any polynucleotide that shows transcriptional activity in the host cell including mutant, truncated, and hybrid promoters, and may be obtained from genes encoding extracellular or intracellular polypeptides either homologous or heterologous to the host cell.

Examples of suitable promoters for directing transcription of the nucleic acid constructs in a bacterial host cell are the promoters obtained from the Bacillus amyloliquefaciens alpha-amylase gene (amyQ), Bacillus licheniformis alpha-amylase gene (amyL), Bacillus licheniformis penicillinase gene (penP), Bacillus stearothermophilus maltogenic amylase gene (amyM), Bacillus subtilis levansucrase gene (sacB), Bacillus subtilis xylA and xylB genes, Bacillus thuringiensis cryIIIA gene (Agaisse and Lereclus, 1994, Molecular Microbiology 13: 97-107), E. coli lac operon, E. coli trc promoter (Egon et al., 1988, Gene 69: 301-315), Streptomyces coelicolor agarase gene (dagA), and prokaryotic beta-lactamase gene (Villa-Kamaroff et al., 1978, Proc. Natl. Acad. Sci. USA 75: 3727-3731), as well as the tac promoter (DeBoer et al., 1983, Proc. Natl. Acad. Sci. USA 80: 21-25). Further promoters are described in “Useful proteins from recombinant bacteria” in Gilbert et al., 1980, Scientific American 242: 74-94; and in Sambrook et al., 1989, supra. Examples of tandem promoters are disclosed in WO 99/43835.

Examples of suitable promoters for directing transcription of the nucleic acid constructs in a filamentous fungal host cell are promoters obtained from the genes for Aspergillus nidulans acetamidase, Aspergillus niger neutral alpha-amylase, Aspergillus niger acid stable alpha-amylase, Aspergillus niger or Aspergillus awamori glucoamylase (glaA), Aspergillus oryzae TAKA amylase, Aspergillus oryzae alkaline protease, Aspergillus oryzae triose phosphate isomerase, Fusarium oxysporum trypsin-like protease (WO 96/00787), Fusarium venenatum amyloglucosidase (WO 00/56900), Fusarium venenatum Dana (WO 00/56900), Fusarium venenatum Quinn (WO 00/56900), Rhizomucor miehei lipase, Rhizomucor miehei aspartic proteinase, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor, as well as the NA2-tpi promoter (a modified promoter from an Aspergillus neutral alpha-amylase gene in which the untranslated leader has been replaced by an untranslated leader from an Aspergillus triose phosphate isomerase gene; non-limiting examples include modified promoters from an Aspergillus niger neutral alpha-amylase gene in which the untranslated leader has been replaced by an untranslated leader from an Aspergillus nidulans or Aspergillus oryzae triose phosphate isomerase gene); and mutant, truncated, and hybrid promoters thereof. Other promoters are described in U.S. Pat. No. 6,011,147.

In a yeast host, useful promoters are obtained from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH1, ADH2/GAP), Saccharomyces cerevisiae triose phosphate isomerase (TPI), Saccharomyces cerevisiae metallothionein (CUP1), and Saccharomyces cerevisiae 3-phosphoglycerate kinase. Other useful promoters for yeast host cells are described by Romanos et al., 1992, Yeast 8: 423-488.

The control sequence may also be a transcription terminator, which is recognized by a host cell to terminate transcription. The terminator is operably linked to the 3′-terminus of the polynucleotide encoding the xyloglucan endotransglycosylase. Any terminator that is functional in the host cell may be used in the present invention.

Preferred terminators for bacterial host cells are obtained from the genes for Bacillus clausii alkaline protease (aprH), Bacillus licheniformis alpha-amylase (amyL), and Escherichia coli ribosomal RNA (rrnB).

Preferred terminators for filamentous fungal host cells are obtained from the genes for Aspergillus nidulans acetamidase, Aspergillus nidulans anthranilate synthase, Aspergillus niger glucoamylase, Aspergillus niger alpha-glucosidase, Aspergillus oryzae TAKA amylase, Fusarium oxysporum trypsin-like protease, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase Ill, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor.

Preferred terminators for yeast host cells are obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, supra.

The control sequence may also be an mRNA stabilizer region downstream of a promoter and upstream of the coding sequence of a gene which increases expression of the gene.

Examples of suitable mRNA stabilizer regions are obtained from a Bacillus thuringiensis cryIIIA gene (WO 94/25612) and a Bacillus subtilis SP82 gene (Hue et al., 1995, Journal of Bacteriology 177: 3465-3471).

The control sequence may also be a leader, a nontranslated region of an mRNA that is important for translation by the host cell. The leader is operably linked to the 5′-terminus of the polynucleotide encoding the xyloglucan endotransglycosylase. Any leader that is functional in the host cell may be used.

Preferred leaders for filamentous fungal host cells are obtained from the genes for Aspergillus oryzae TAKA amylase and Aspergillus nidulans triose phosphate isomerase.

Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′-terminus of the polynucleotide and, when transcribed, is recognized by the host cell as a signal to add polyadenosine residues to transcribed mRNA. Any polyadenylation sequence that is functional in the host cell may be used.

Preferred polyadenylation sequences for filamentous fungal host cells are obtained from the genes for Aspergillus nidulans anthranilate synthase, Aspergillus niger glucoamylase, Aspergillus niger alpha-glucosidase Aspergillus oryzae TAKA amylase, and Fusarium oxysporum trypsin-like protease.

Useful polyadenylation sequences for yeast host cells are described by Guo and Sherman, 1995, Mol. Cellular Biol. 15: 5983-5990.

The control sequence may also be a signal peptide coding region that encodes a signal peptide linked to the N-terminus of a xyloglucan endotransglycosylase and directs the polypeptide into the cell's secretory pathway. The 5′-end of the coding sequence of the polynucleotide may inherently contain a signal peptide coding sequence naturally linked in translation reading frame with the segment of the coding sequence that encodes the polypeptide. Alternatively, the 5′-end of the coding sequence may contain a signal peptide coding sequence that is foreign to the coding sequence. A foreign signal peptide coding sequence may be required where the coding sequence does not naturally contain a signal peptide coding sequence. Alternatively, a foreign signal peptide coding sequence may simply replace the natural signal peptide coding sequence in order to enhance secretion of the polypeptide. However, any signal peptide coding sequence that directs the expressed polypeptide into the secretory pathway of a host cell may be used.

Effective signal peptide coding sequences for bacterial host cells are the signal peptide coding sequences obtained from the genes for Bacillus NCIB 11837 maltogenic amylase, Bacillus licheniformis subtilisin, Bacillus licheniformis beta-lactamase, Bacillus stearothermophilus alpha-amylase, Bacillus stearothermophilus neutral proteases (nprT, nprS, nprM), and Bacillus subtilis prsA. Further signal peptides are described by Simonen and Palva, 1993, Microbiological Reviews 57: 109-137.

Effective signal peptide coding sequences for filamentous fungal host cells are the signal peptide coding sequences obtained from the genes for Aspergillus niger neutral amylase, Aspergillus niger glucoamylase, Aspergillus oryzae TAKA amylase, Humicola insolens cellulase, Humicola insolens endoglucanase V, Humicola lanuginosa lipase, and Rhizomucor miehei aspartic proteinase.

Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra.

The control sequence may also be a propeptide coding sequence that encodes a propeptide positioned at the N-terminus of a xyloglucan endotransglycosylase. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of a xyloglucan endotransglycosylase and the signal peptide sequence is positioned next to the N-terminus of the propeptide sequence.

The various nucleotide and control sequences may be joined together to produce a recombinant expression vector that may include one or more convenient restriction sites to allow for insertion or substitution of the polynucleotide encoding the xyloglucan endotransglycosylase at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated.

Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Examples of bacterial selectable markers are Bacillus licheniformis or Bacillus subtilis dal genes, or markers that confer antibiotic resistance such as ampicillin, chloramphenicol, kanamycin, neomycin, spectinomycin, or tetracycline resistance. Suitable markers for yeast host cells include, but are not limited to, ADE2, HIS3, LEU2, LYS2, MET3, TRP1, and URA3. Selectable markers for use in a filamentous fungal host cell include, but are not limited to, adeA (phosphoribosylaminoimidazole-succinocarboxamide synthase), adeB (phosphoribosyl-aminoimidazole synthase), amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergillus cell are Aspergillus nidulans or Aspergillus oryzae amdS and pyrG genes and a Streptomyces hygroscopicus bar gene. Preferred for use in a Trichoderma cell are adeA, adeB, amdS, hph, and pyrG genes.

The selectable marker may be a dual selectable marker system as described in WO 2010/039889. In one aspect, the dual selectable marker is an hph-tk dual selectable marker system.

The vector preferably contains an element(s) that permits integration of the vector into the host cell's genome or autonomous replication of the vector in the cell independent of the genome.

For integration into the host cell genome, the vector may rely on the polynucleotide's sequence encoding the xyloglucan endotransglycosylase or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of bacterial origins of replication are the origins of replication of plasmids pBR322, pUC19, pACYC177, and pACYC184 permitting replication in E. coli, and pUB110, pE194, pTA1060, and pAMR1 permitting replication in Bacillus.

Examples of origins of replication for use in a yeast host cell are the 2 micron origin of replication, ARS1, ARS4, the combination of ARS1 and CEN3, and the combination of ARS4 and CEN6.

Examples of origins of replication useful in a filamentous fungal cell are AMA1 and ANSI (Gems et al., 1991, Gene 98: 61-67; Cullen et al., 1987, Nucleic Acids Res. 15: 9163-9175; WO 00/24883). Isolation of the AMA1 gene and construction of plasmids or vectors comprising the gene can be accomplished according to the methods disclosed in WO 00/24883.

More than one copy of a polynucleotide may be inserted into a host cell to increase production of a xyloglucan endotransglycosylase. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors are well known to one skilled in the art (see, e.g., Sambrook et al., 1989, supra).

The host cell may be any cell useful in the recombinant production of a xyloglucan endotransglycosylase, e.g., a prokaryote or a eukaryote.

The prokaryotic host cell may be any Gram-positive or Gram-negative bacterium. Gram-positive bacteria include, but are not limited to, Bacillus, Clostridium, Enterococcus, Geobacillus, Lactobacillus, Lactococcus, Oceanobacillus, Staphylococcus, Streptococcus, and Streptomyces. Gram-negative bacteria include, but are not limited to, Campylobacter, E. coli, Flavobacterium, Fusobacterium, Helicobacter, Ilyobacter, Neisseria, Pseudomonas, Salmonella, and Ureaplasma.

The bacterial host cell may be any Bacillus cell including, but not limited to, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis cells.

The bacterial host cell may also be any Streptomyces cell including, but not limited to, Streptomyces achromogenes, Streptomyces avermitilis, Streptomyces coelicolor, Streptomyces griseus, and Streptomyces lividans cells.

The introduction of DNA into a Bacillus cell may be effected by protoplast transformation (see, e.g., Chang and Cohen, 1979, Mol. Gen. Genet. 168: 111-115), competent cell transformation (see, e.g., Young and Spizizen, 1961, J. Bacteriol. 81: 823-829, or Dubnau and Davidoff-Abelson, 1971, J. Mol. Biol. 56: 209-221), electroporation (see, e.g., Shigekawa and Dower, 1988, Biotechniques 6: 742-751), or conjugation (see, e.g., Koehler and Thorne, 1987, J. Bacteriol. 169: 5271-5278). The introduction of DNA into an E. coli cell may be effected by protoplast transformation (see, e.g., Hanahan, 1983, J. Mol. Biol. 166: 557-580) or electroporation (see, e.g., Dower et al., 1988, Nucleic Acids Res. 16: 6127-6145). The introduction of DNA into a Streptomyces cell may be effected by protoplast transformation, electroporation (see, e.g., Gong et al., 2004, Folia Microbiol. (Praha) 49: 399-405), conjugation (see, e.g., Mazodier et al., 1989, J. Bacteriol. 171: 3583-3585), or transduction (see, e.g., Burke et al., 2001, Proc. Natl. Acad. Sci. USA 98: 6289-6294). The introduction of DNA into a Pseudomonas cell may be effected by electroporation (see, e.g., Choi et al., 2006, J. Microbiol. Methods 64: 391-397) or conjugation (see, e.g., Pinedo and Smets, 2005, Appl. Environ. Microbiol. 71: 51-57). The introduction of DNA into a Streptococcus cell may be effected by natural competence (see, e.g., Perry and Kuramitsu, 1981, Infect. Immun. 32: 1295-1297), protoplast transformation (see, e.g., Catt and Jollick, 1991, Microbios 68: 189-207), electroporation (see, e.g., Buckley et al., 1999, Appl. Environ. Microbiol. 65: 3800-3804), or conjugation (see, e.g., Clewell, 1981, Microbiol. Rev. 45: 409-436). However, any method known in the art for introducing DNA into a host cell can be used.

The host cell may also be a eukaryote, such as a mammalian, insect, plant, or fungal cell.

The host cell may be a fungal cell. “Fungi” as used herein includes the phyla Ascomycota, Basidiomycota, Chytridiomycota, and Zygomycota as well as the Oomycota and all mitosporic fungi (as defined by Hawksworth et al., In, Ainsworth and Bisby's Dictionary of The Fungi, 8th edition, 1995, CAB International, University Press, Cambridge, UK).

The fungal host cell may be a yeast cell. “Yeast” as used herein includes ascosporogenous yeast (Endomycetales), basidiosporogenous yeast, and yeast belonging to the Fungi lmperfecti (Blastomycetes). Since the classification of yeast may change in the future, for the purposes of this invention, yeast shall be defined as described in Biology and Activities of Yeast (Skinner, Passmore, and Davenport, editors, Soc. App. Bacteriol. Symposium Series No. 9, 1980).

The yeast host cell may be a Candida, Hansenula, Kluyveromyces, Pichia, Saccharomyces, Schizosaccharomyces, or Yarrowia cell, such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell.

The fungal host cell may be a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238023, Yelton et al., 1984, Proc. Natl. Acad. Sci. USA 81: 1470-1474, and Christensen et al., 1988, Bio/Technology 6: 1419-1422. Suitable methods for transforming Fusarium species are described by Malardier et al., 1989, Gene 78: 147-156, and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp. 182-187, Academic Press, Inc., New York; Ito et al., 1983, J. Bacteriol. 153: 163; and Hinnen et al., 1978, Proc. Natl. Acad. Sci. USA 75: 1920.

The host cells are cultivated in a nutrient medium suitable for production of the xyloglucan endotransglycosylase using methods known in the art. For example, the cells may be cultivated by shake flask cultivation, or small-scale or large-scale fermentation (including continuous, batch, fed-batch, or solid state fermentations) in laboratory or industrial fermentors in a suitable medium and under conditions allowing the xyloglucan endotransglycosylase to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the xyloglucan endotransglycosylase is secreted into the nutrient medium, the polypeptide can be recovered directly from the medium. If the xyloglucan endotransglycosylase is not secreted, it can be recovered from cell lysates.

The xyloglucan endotransglycosylase may be detected using methods known in the art that are specific for the polypeptides. These detection methods include, but are not limited to, use of specific antibodies, formation of an enzyme product, or disappearance of an enzyme substrate. For example, an enzyme assay may be used to determine the activity of the polypeptide.

The xyloglucan endotransglycosylase may be recovered using methods known in the art. For example, the polypeptide may be recovered from the nutrient medium by conventional procedures including, but not limited to, collection, centrifugation, filtration, extraction, spray-drying, evaporation, or precipitation. In one aspect, a whole fermentation broth comprising the polypeptide is recovered. In a preferred aspect, xyloglucan endotransglycosylase yield may be improved by subsequently washing cellular debris in buffer or in buffered detergent solution to extract biomass-associated polypeptide.

The xyloglucan endotransglycosylase may be purified by a variety of procedures known in the art including, but not limited to, chromatography (e.g., ion exchange, affinity, hydrophobic interaction, mixed mode, reverse phase, chromatofocusing, and size exclusion), electrophoretic procedures (e.g., preparative isoelectric focusing), differential solubility (e.g., ammonium sulfate precipitation), PAGE, membrane-filtration or extraction (see, e.g., Protein Purification, Janson and Ryden, editors, VCH Publishers, New York, 1989) to obtain substantially pure polypeptide. In a preferred aspect, xyloglucan endotransglycosylase may be purified by formation of a covalent acyl-enzyme intermediate with xyloglucan, followed by precipitation with microcrystalline cellulose or adsorption to cellulose membranes. Release of the polypeptide is then effected by addition of xyloglucan oligomers to resolve the covalent intermediate (Sulova and Farkas, 1999, Protein Expression and Purification 16(2): 231-235, and Steele and Fry, 1999, Biochemical Journal 340: 207-211).

The present invention is further described by the following examples that should not be construed as limiting the scope of the invention.

### EXAMPLES

### Media and Solutions

COVE agar plates were composed of 342.3 g of sucrose, 252.54 g of CsCl, 59.1 g of acetamide, 520 mg of KCl, 520 mg of MgSO₄7H₂O, 1.52 g of KH₂PO₄, 0.04 mg of Na₂B₄O₇.10H₂O, 0.4 mg of CuSO₄5H₂O, 1.2 mg of FeSO₄7H₂O, 0.7 mg of MnSO₄2H₂O, 0.8 mg of Na₂MoO₄.2H₂O, 10 mg of ZnSO₄7H₂O, 25 g of Noble agar, and deionized water to 1 liter.

LB medium was composed of 10 g of tryptone, 5 g of yeast extract, 5 g of NaCl, and deionized water to 1 liter.

LB plates were composed of 10 g of tryptone, 5 g of yeast extract, 5 g of NaCl, 15 g of bacteriological agar, and deionized water to 1 liter.

Minimal medium agar plates were composed of 342.3 g of sucrose, 10 g of glucose, 4 g of MgSO₄7H₂O, 6 g of NaNO₃, 0.52 g of KCl, 1.52 g of KH₂PO₄, 0.04 mg of Na₂B₄O₇.10H₂O, 0.4 mg of CuSO₄5H₂O, 1.2 mg of FeSO₄7H₂O, 0.7 mg of MnSO₄2H₂O, 0.8 mg of Na₂MoO₄.2H₂O, 10 mg of ZnSO₄7H₂O, 500 mg of citric acid, 4 mg of d-biotin, 20 g of Noble agar, and deionized water to 1 liter.

Spizizen I medium was composed of 1× Spizizen salts, 0.5% glucose, 0.1% yeast extract, and 0.02% casein hydrolysate in deionized water.

1× Spizizen salts was composed of 6 g of KH₂PO₄, 14 g of K₂HPO₄, 2 g of (NH₄)₂SO₄, 1 g of sodium citrate, 0.2 g of MgSO₄, and deionized water to 1 liter; pH 7.0.

Synthetic defined medium lacking uridine was composed of 18 mg of adenine hemisulfate, 76 mg of alanine, 76 mg of arginine hydrochloride, 76 mg of asparagine monohydrate, 76 mg of aspartic acid, 76 mg of cysteine hydrochloride monohydrate, 76 mg of glutamic acid monosodium salt, 76 mg of glutamine, 76 mg of glycine, 76 mg of histidine, myo-76 mg of inositol, 76 mg of isoleucine, 380 mg of leucine, 76 mg of lysine monohydrochloride, 76 mg of methionine, 8 mg of p-aminobenzoic acid potassium salt, 76 mg of phenylalanine, 76 mg of proline, 76 mg of serine, 76 mg of threonine, 76 mg of tryptophan, 76 mg of tyrosine disodium salt, 76 mg of valine, and deionized water to 1 liter.

TAE buffer was composed of 4.84 g of Tris base, 1.14 ml of glacial acetic acid, 2 ml of 0.5 M EDTA pH 8.0, and deionized water to 1 liter.

TBAB plates were composed of 33 g of Tryptose Blood Agar Base (Difco Laboratories, Sparks, Md., USA) and deionized water to 1 liter.

TBE buffer was composed of 10.8 g of Tris base, 5.5 g of boric acid, 4 ml of 0.5 M EDTA pH 8.0, and deionized water to 1 liter.

2×YT plus ampicillin plates were composed of 16 g of tryptone, 10 g of yeast extract, 5 g of sodium chloride, 15 g of Bacto agar, and deionized water to 1 liter. One ml of a 100 mg/ml solution of ampicillin was added after the autoclaved medium was tempered to 55° C.

Yeast ura minus selection medium was composed of 6.7 g of yeast nitrogen base (YNB) with ammonium sulfate, 5 g of Casamino acids, 100 ml of 0.5 M succinic acid pH 5, 40 ml of 50% glucose, 2 ml of 10 mg/ml chloramphenicol, and deionized water to 1 liter.

Yeast ura minus selection plates were composed of yeast ura minus selection medium supplemented with 20 g of Noble agar per liter.

YP+2% glucose medium was composed of 10 g of yeast extract, 20 g of peptone, 20 g of glucose, and deionized water to 1 liter.

YP+2% maltodextrin medium was composed of 10 g of yeast extract, 20 g of peptone, 20 g of maltodextrin, and deionized water to 1 liter.

### Example 1

### Preparation of Vigna angularis Xyloglucan Endotransglycosylase 16

Vigna angularis xyloglucan endotransglycosylase 16 (VaXET16; SEQ ID NO: 1 [native DNA sequence], SEQ ID NO: 2 [synthetic DNA sequence], and SEQ ID NO: 3 [deduced amino acid sequence]; also referred to as XTH1) was recombinantly produced in Aspergillus oryzae MT3568 according to the protocol described below. Aspergillus oryzae MT3568 is an amdS (acetamidase) disrupted gene derivative of Aspergillus oryzae JaL355 (WO 2002/40694), in which pyrG auxotrophy was restored by disrupting the A. oryzae amdS gene with the pyrG gene.

The vector pDLHD0012 was constructed to express the VaXET16 gene in multi-copy in Aspergillus oryzae. Plasmid pDLHD0012 was generated by combining two DNA fragments using megaprimer cloning: Fragment 1 containing the VaXET16 ORF and flanking sequences with homology to vector pBM120 (US20090253171), and Fragment 2 consisting of an inverse PCR amplicon of vector pBM120.

Fragment 1 was amplified using primer 613788 (sense) and primer 613983 (antisense) shown below. These primers were designed to contain flanking regions of sequence homology to vector pBM120 (lower case) for ligation-free cloning between the PCR fragments.

Primer 613788 (sense):
(SEQ ID NO: 7)
ttcctcaatcctctatatacacaactggccATGGGCTCGTCCCTCTGGAC
Primer 613983 (antisense):
(SEQ ID NO: 8)
tgtcagtcacctctagttaattaGATGTCCCTATCGCGTGTACACTCG

Fragment 1 was amplified by PCR in a reaction composed of 10 ng of a GENEART® vector pMA containing the VaXET16 synthetic gene (SEQ ID NO: 3 [synthetic DNA sequence]) cloned between the Sac I and Kpn I sites, 0.5 μl of PHUSION® DNA Polymerase (New England Biolabs, Inc., Ipswich, Mass., USA), 20 pmol of primer 613788, 20 pmol of primer 613983, 1 μl of 10 mM dNTPs, 10 μl of 5× PHUSION® HF buffer (New England Biolabs, Inc., Ipswich, Mass., USA), and 35.5 μl of water. The reaction was incubated in an EPPENDORF® MASTERCYCLER® (Eppendorf AG, Hamburg, Germany) programmed for 1 cycle at 98° C. for 30 seconds; and 30 cycles each at 98° C. for 10 seconds, 60° C. for 10 seconds, and 72° C. for 30 seconds. The resulting 0.9 kb PCR product (Fragment 1) was treated with 1 μl of Dpn I (Promega, Fitchburg, Wis., USA) to remove plasmid template DNA. The Dpn I was added directly to the PCR tube, mixed well, and incubated at 3TC for 60 minutes, and then was column-purified using a MINELUTE® PCR Purification Kit (QIAGEN Inc., Valencia, Calif., USA) according to the manufacturer's instructions.

Fragment 2 was amplified using primers 613786 (sense) and 613787 (antisense) shown below.

613786 (sense):
(SEQ ID NO: 9)
taattaactagaggtgactgacacctggc
613787 (antisense):
(SEQ ID NO: 10)
catggccagttgtgtatatagaggattgagg

Fragment 2 was amplified by PCR in a reaction composed of 10 ng of plasmid pBM120, 0.5 μl of PHUSION® DNA Polymerase, 20 pmol of primer 613786, 20 pmol of primer 613787, 1 μl of 10 mM dNTPs, 10 μl of 5× PHUSION® HF buffer, and 35.5 μl of water. The reaction was incubated in an EPPENDORF® MASTERCYCLER® programmed for 1 cycle at 98° C. for 30 seconds; and 30 cycles each at 98° C. for 10 seconds, 60° C. for 10 seconds, and 72° C. for 4 minutes. The resulting 6.9 kb PCR product (Fragment 2) was treated with 1 μl of Dpn I to remove plasmid template DNA. The Dpn I was added directly to the PCR tube, mixed well, and incubated at 3TC for 60 minutes, and then column-purified using a MINELUTE® PCR Purification Kit according to the manufacturer's instructions.

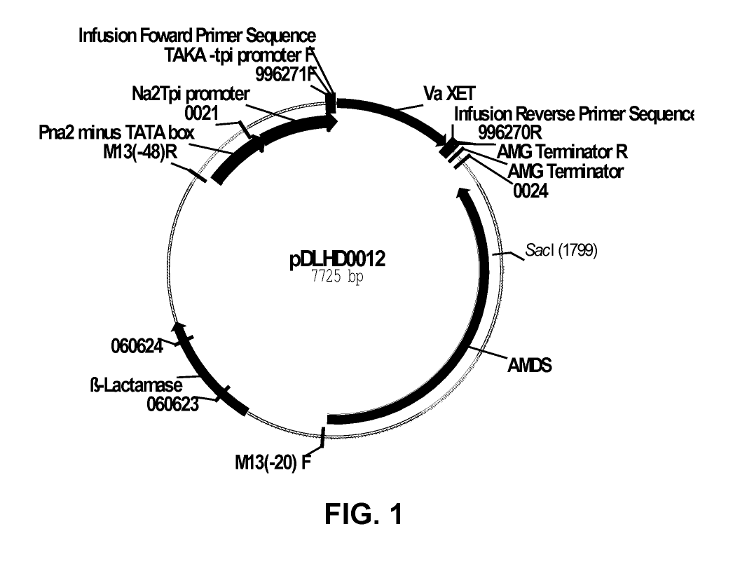

The following procedure was used to combine the two PCR fragments using megaprimer cloning. Fragments 1 and 2 were combined by PCR in a reaction composed of 5 μl of each purified PCR product, 0.5 μl of PHUSION® DNA Polymerase, 1 μl of 10 mM dNTPs, 10 μl of 5× PHUSION® HF buffer, and 28.5 μl of water. The reaction was incubated in an EPPENDORF® MASTERCYCLER® programmed for 1 cycle at 98° C. for 30 seconds; and 40 cycles each at 98° C. for 10 seconds, 60° C. for 10 seconds, and 72° C. for 4 minutes. Two μl of the resulting PCR product DNA was then transformed into E. coli ONE SHOT® TOP10 electrocompetent cells (Life Technologies, Grand Island, N.Y., USA) according the manufacturer's instructions. Fifty μl of transformed cells were spread onto LB plates supplemented with 100 μg of ampicillin per ml and incubated at 3TC overnight. Individual transformants were picked into 3 ml of LB medium supplemented with 100 μg of ampicillin per ml and grown overnight at 3TC with shaking at 250 rpm. The plasmid DNA was purified from the colonies using a QIAPREP® Spin Miniprep Kit (QIAGEN Inc., Valencia, Calif., USA). DNA sequencing using a 3130XL Genetic Analyzer (Applied Biosystems, Foster City, Calif., USA) was used to confirm the presence of each of both fragments in the final plasmid pDLHD0012 (FIG. 1).

Aspergillus oryzae strain MT3568 was transformed with plasmid pDLHD0012 comprising the VaXET16 gene according to the following protocol. Approximately 2-5×10⁷ spores of A. otyzae strain MT3568 were inoculated into 100 ml of YP+2% glucose medium in a 500 ml shake flask and incubated at 28° C. and 110 rpm overnight. Ten ml of the overnight culture were filtered in a 125 ml sterile vacuum filter, and the mycelia were washed twice with 50 ml of 0.7 M KCl-20 mM CaCl₂. The remaining liquid was removed by vacuum filtration, leaving the mat on the filter. Mycelia were resuspended in 10 ml of 0.7 M KCl-20 mM CaCl₂ and transferred to a sterile 125 ml shake flask containing 20 mg of GLUCANEX® 200 G (Novozymes Switzerland AG, Neumatt, Switzerland) per ml and 0.2 mg of chitinase (Sigma-Aldrich, St. Louis, Mo., USA) per ml in 10 ml of 0.7 M KCl-20 mM CaCl₂. The mixture was incubated at 37° C. and 100 rpm for 30-90 minutes until protoplasts were generated from the mycelia. The protoplast mixture was filtered through a sterile funnel lined with MIRACLOTH® (Calbiochem, San Diego, Calif., USA) into a sterile 50 ml plastic centrifuge tube to remove mycelial debris. The debris in the MIRACLOTH® was washed thoroughly with 0.7 M KCl-20 mM CaCl₂ and centrifuged at 2500 rpm (537×g) for 10 minutes at 20-23° C. The supernatant was removed and the protoplast pellet was resuspended in 20 ml of 1 M sorbitol-10 mM Tris-HCl (pH 6.5)-10 mM CaCl₂. This step was repeated twice, and the final protoplast pellet was resuspended in 1 M sorbitol-10 mM Tris-HCl (pH 6.5)-10 mM CaCl₂ to obtain a final protoplast concentration of 2×10⁷/ml.

Two micrograms of pDLHD0012 were added to the bottom of a sterile 2 ml plastic centrifuge tube. Then 100 μl of protoplasts were added to the tube followed by 300 μl of 60% PEG-4000 in 10 mM Tris-HCl (pH 6.5)-10 mM CaCl₂. The tube was mixed gently by hand and incubated at 37° C. for 30 minutes. Two ml of 1 M sorbitol-10 mM Tris-HCl (pH 6.5)-10 mM CaCl₂ were added to each transformation and the mixture was transferred onto 150 mm COVE agar plates. Transformation plates were incubated at 34° C. until colonies appeared.

Twenty-one transformant colonies were picked to fresh COVE agar plates and cultivated at 34° C. for four days until the transformants sporulated. Fresh spores were transferred to 48-well deep-well plates containing 2 ml of YP+2% maltodextrin, covered with a breathable seal, and grown for 4 days at 34° C. with no shaking. After 4 days growth samples of the culture media were assayed for xyloglucan endotransglycosylase activity using an iodine stain assay and for xyloglucan endotransglycosylase expression by SDS-PAGE.

The iodine stain assay for xyloglucan endotransglycosylase activity was performed according to the following protocol. In a 96-well plate, 5 μl of culture broth were added to a mixture of 5 μl of xyloglucan (Megazyme, Bray, United Kingdom) (5 mg/ml in water), 20 μl of xyloglucan oligomers (Megazyme, Bray, United Kingdom) (5 mg/ml in water), and 10 μl of 400 mM sodium citrate pH 5.5. The reaction mix was incubated at 37° C. for thirty minutes, quenched with 200 μl of a solution containing 14% (w/v) Na₂SO₄, 0.2% KI, 100 mM HCl, and 1% iodine (I₂), incubated in the dark for 30 minutes, and then the absorbance was measured in a plate reader at 620 nm. The assay demonstrated the presence of xyloglucan endotransglycosylase activity from several transformants.

SDS-PAGE was performed using a β-16% CRITERION® Stain Free SDS-PAGE gel (Bio-Rad Laboratories, Inc., Hercules, Calif., USA), and imaging the gel with a Stain Free Imager (Bio-Rad Laboratories, Inc., Hercules, Calif., USA) using the following settings: 5-minute activation, automatic imaging exposure (intense bands), highlight saturated pixels=ON, color=Coomassie, and band detection, molecular weight analysis and reporting disabled. SDS-PAGE analysis indicated that several transformants expressed a protein of approximately 32 kDa corresponding to VaXET16.

### Example 2

### Construction of Plasmid pMMar27 as a Yeast Expression Plasmid Vector

Plasmid pMMar27 was constructed for expression of the T. terrestris Cel6A cellobiohydrolase 11 in yeast. The plasmid was generated from a lineage of yeast expression vectors: plasmid pMMar27 was constructed from plasmid pBM175b; plasmid pBM175b was constructed from plasmid pBM143b (WO 2008/008950) and plasmid pJLin201; and plasmid pJLin201 was constructed from pBM143b.

Plasmid pJLin201 is identical to pBM143b except an Xba I site immediately downstream of a Thermomyces lanuginosus lipase variant gene in pBM143b was mutated to a unique Nhe I site. A QUIKCHANGE® 11 XL Site-Directed Mutagenesis Kit (Stratagene, La Jolla, Calif., USA) was used to change the Xba 1 sequence (TCTAGA) to a Nhe 1 sequence (gCTAGc) in pBM143b. Primers employed to mutate the site are shown below.

Primer 999551 (sense):
(SEQ ID NO: 11)
5′-ACATGTCTTTGATAAgCTAGcGGGCCGCATCATGTA-3′
Primer 999552 (antisense):
(SEQ ID NO: 12)
5′-TACATGATGCGGCCCgCTAGcTTATCAAAGACATGT-3′

Lower case represents mutated nucleotides.

The amplification reaction was composed of 125 ng of each primer above, 20 ng of pBM143b, 1× QUIKCHANGE® Reaction Buffer (Stratagene, La Jolla, Calif., USA), 3 μl of QUIKSOLUTION® (Stratagene, La Jolla, Calif., USA), 1 μl of dNTP mix, and 1 μl of a 2.5 units/ml Pfu Ultra HF DNA polymerase in a final volume of 50 μl. The reaction was performed using an EPPENDORF® MASTERCYCLER® thermocycler programmed for 1 cycle at 95° C. for 1 minute; 18 cycles each at 95° C. for 50 seconds, 60° C. for 50 seconds, and 68° C. for 6 minutes and 6 seconds; and 1 cycle at 68° C. for 7 minutes. After the PCR, the tube was placed on ice for 2 minutes. One microliter of Dpn I was directly added to the amplification reaction and incubated at 37° C. for 1 hour. A 2 μl volume of the Dpn I digested reaction was used to transform E. coli XL10-GOLD® Ultracompetent Cells (Stratagene, La Jolla, Calif., USA) according to the manufacturer's instructions. E. coli transformants were selected on 2×YT plus ampicillin plates. Plasmid DNA was isolated from several of the transformants using a BIOROBOT® 9600. One plasmid with the desired Nhe I change was confirmed by restriction digestion and sequencing analysis and designated plasmid pJLin201. To eliminate possible PCR errors introduced by site-directed-mutagenesis, plasmid pBM175b was constructed by cloning the Nhe I site containing fragment back into plasmid pBM143b. Briefly, plasmid pJLin201 was digested with Nde I and Mu I and the resulting fragment was cloned into pBM143b previously digested with the same enzymes using a Rapid Ligation Kit (Roche Diagnostics Corporation, Indianapolis, Ind., USA). Then, 7 μl of the Nde I/MIu I digested pJLin201 fragment and 1 μl of the digested pBM143b were mixed with 2 μl of 5×DNA dilution buffer (Roche Diagnostics Corporation, Indianapolis, Ind., USA), 10 μl of 2×T4 DNA ligation buffer (Roche Diagnostics Corporation, Indianapolis, Ind., USA), and 1 μl of T4 DNA ligase (Roche Diagnostics Corporation, Indianapolis, Ind., USA) and incubated for 15 minutes at room temperature. Two microliters of the ligation were transformed into XL1-Blue Subcloning-Grade Competent Cells (Stratagene, La Jolla, Calif., USA) cells and spread onto 2×YT plus ampicillin plates. Plasmid DNA was purified from several transformants using a BIOROBOT® 9600 and analyzed by DNA sequencing using a 3130XL Genetic Analyzer to identify a plasmid containing the desired A. nidulans pyrG insert. One plasmid with the expected DNA sequence was designated pBM175b.

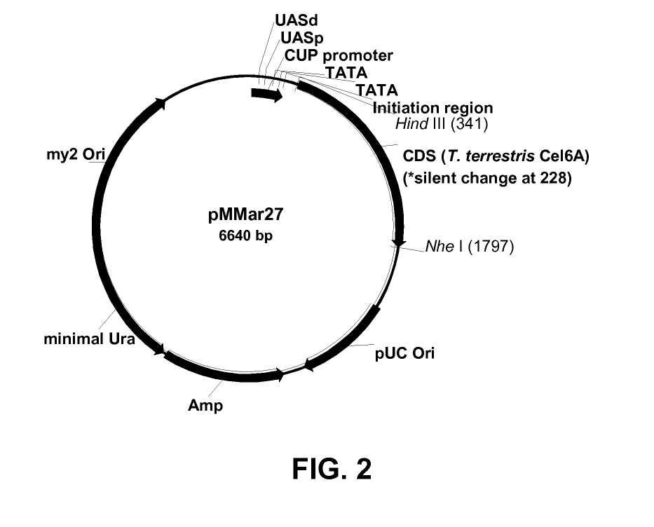

Plasmid pMMar27 was constructed from pBM175b and an amplified gene of T. terrestris Cel6A cellobiohydrolase II with overhangs designed for insertion into digested pBM175b. Plasmid pBM175b containing the Thermomyces lanuginosus lipase variant gene under control of the CUP I promoter contains unique Hind III and Nhe I sites to remove the lipase gene. Plasmid pBM175 was digested with these restriction enzymes to remove the lipase gene. After digestion, the empty vector was isolated by 1.0% agarose gel electrophoresis using TBE buffer where an approximately 5,215 bp fragment was excised from the gel and extracted using a QIAQUICK® Gel Extraction Kit. The ligation reaction (20 μl) was composed of 1× IN-FUSION® Buffer (BD Biosciences, Palo Alto, Calif., USA), 1×BSA (BD Biosciences, Palo Alto, Calif., USA), 1 μl of IN-FUSION® enzyme (diluted 1:10) (BD Biosciences, Palo Alto, Calif., USA), 99 ng of pBM175b digested with Hind III and Nhe I, and 36 ng of the purified T. terrestris Cel6A cellobiohydrolase II PCR product. The reaction was incubated at room temperature for 30 minutes. A 2 μl volume of the IN-FUSION® reaction was transformed into E. coli XL10-GOLD® Ultracompetent Cells. Transformants were selected on LB plates supplemented with 100 μg of ampicillin per ml. A colony was picked that contained the T. terrestris Cel6A inserted into the pBM175b vector in place of the lipase gene, resulting in pMMar27 (FIG. 2). The plasmid chosen contained a PCR error at position 228 from the start codon, TCT instead of TCC, but resulted in a silent change in the T. terrestris Cel6A cellobiohydrolase II.

### Example 3

### Construction of pEvFz1 Expression Vector

Expression vector pEvFz1 was constructed by modifying pBM120a (U.S. Pat. No. 8,263,824) to comprise the NA2/NA2-tpi promoter, A. niger amyloglucosidase terminator sequence (AMG terminator), and Aspergillus nidulans orotidine-5′-phosphate decarboxylase gene (pyrG) as a selectable marker.

Plasmid pEvFz1 was generated by cloning the A. nidulans pyrG gene from pAlLo2 (WO 2004/099228) into pBM120a. Plasmids pBM120a and pAlLo2 were digested with Nsi I overnight at 37° C. The resulting 4176 bp linearized pBM120a vector fragment and the 1479 bp pyrG gene insert from pAlLo2 were each purified by 0.7% agarose gel electrophoresis using TAE buffer, excised from the gel, and extracted using a QIAQUICK® Gel Extraction Kit.

The 1479 bp pyrG gene insert was ligated to the Nsi I digested pBM120a fragment using a QUICK LIGATION™ Kit (New England Biolabs, Beverly, Mass., USA). The ligation reaction was composed of 1× QUICK LIGATION™ Reaction Buffer (New England Biolabs, Beverly, Mass., USA), 50 ng of Nsi I digested pBM120a vector, 54 ng of the 1479 bp Nsi I digested pyrG gene insert, and 1 μl of T4 DNA ligase in a total volume of 20 μl. The ligation mixture was incubated at 37° C. for 15 minutes followed at 50° C. for 15 minutes and then placed on ice.

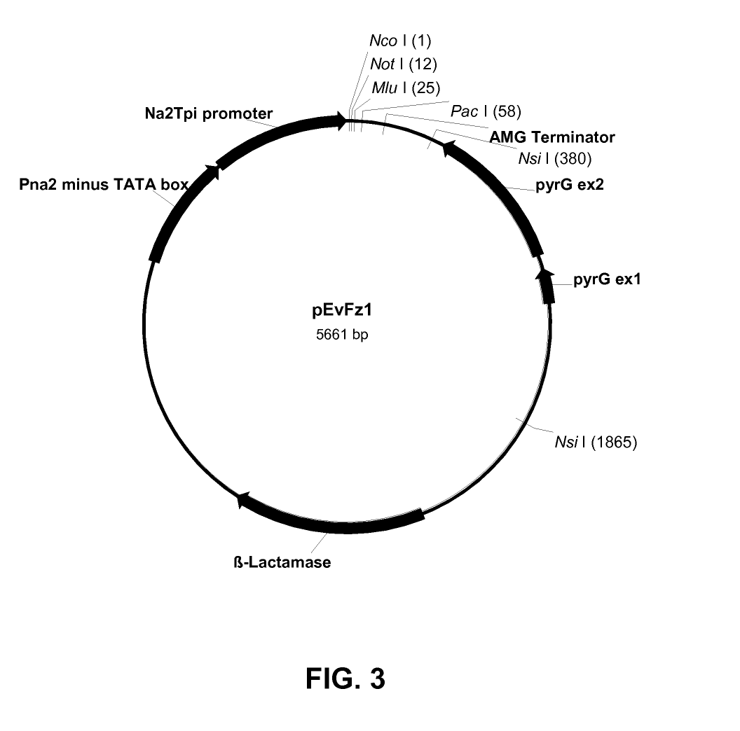

One μl of the ligation mixture was transformed into ONE SHOT® TOP10 chemically competent Escherichia coli cells. Transformants were selected on 2×YT plus ampicillin plates. Plasmid DNA was purified from several transformants using a BIOROBOT® 9600 and analyzed by DNA sequencing using a 3130XL Genetic Analyzer to identify a plasmid containing the desired A. nidulans pyrG insert. One plasmid with the expected DNA sequence was designated pEvFz1 (FIG. 3).

### Example 4

### Construction of the Plasmid pDLHD0006 as a Yeast/E. coli/A. Oryzae Shuttle Vector

Plasmid pDLHD0006 was constructed as a base vector to enable A. oryzae expression cassette library building using yeast recombinational cloning. Plasmid pDLHD0006 was generated by combining three DNA fragments using yeast recombinational cloning: Fragment 1 containing the E. coli pUC origin of replication, E. coli beta-lactamase (ampR) selectable marker, URA3 yeast selectable marker, and yeast 2 micron origin of replication from pMMar27 (Example 2); Fragment 2 containing the 10 amyR/NA2-tpi promoter (a hybrid of the promoters from the genes encoding Aspergillus niger neutral alpha-amylase and Aspergillus oryzae triose phosphate isomerase and including 10 repeated binding sites for the Aspergillus oryzae amyR transcription factor), Thermomyces lanuginosus lipase open reading frame (ORF), and Aspergillus niger glucoamylase terminator from pJaL1262 (WO 2013/178674); and Fragment 3 containing the Aspergillus nidulans pyrG selection marker from pEvFz1 (Example 3).

| pDLHD0006 | PCR Contents | PCR Template |
|---|---|---|
| Fragment 1 | E. coli ori/AmpR/URA/2 micron (4.1 kb) | pMMar27 |
| Fragment 2 | 10 amyR/NA2-tpi PR/lipase/Tamg | pJaL1262 |
| | (4.5 kb) | |
| Fragment 3 | pyrG gene from pEvFz1 (1.7 kb) | pEvFz1 |

Fragment 1 was amplified using primers 613017 (sense) and 613018 (antisense) shown below. Primer 613017 was designed to contain a flanking region with sequence homology to Fragment 3 (lower case) and primer 613018 was designed to contain a flanking region with sequence homology to Fragment 2 (lower case) to enable yeast recombinational cloning between the three PCR fragments.

Primer 613017 (sense):
(SEQ ID NO: 13)
ttaatcgccttgcagcacaCCGCTTCCTCGCTCACTGACTC
613018 (antisense):
(SEQ ID NO: 14)
acaataaccctgataaatgcGGAACAACACTCAACCCTATCTCGGTC

Fragment 1 was amplified by PCR in a reaction composed of 10 ng of plasmid pMMar27, 0.5 μl of PHUSION® DNA Polymerase (New England Biolabs, Inc., Ipswich, Mass., USA), 20 pmol of primer 613017, 20 pmol of primer 613018, 1 μl of 10 mM dNTPs, 10 μl of 5× PHUSION® HF buffer, and 35.5 μl of water. The reaction was incubated in an EPPENDORF® MASTERCYCLER® programmed for 1 cycle at 98° C. for 30 seconds; and 30 cycles each at 98° C. for 10 seconds, 60° C. for 10 seconds, and 72° C. for 1.5 minutes. The resulting 4.1 kb PCR product (Fragment 1) was used directly for yeast recombination with Fragments 2 and 3 below.

Fragment 2 was amplified using primers 613019 (sense) and 613020 (antisense) shown below. Primer 613019 was designed to contain a flanking region of sequence homology to Fragment 1 (lower case) and primer 613020 was designed to contain a flanking region of sequence homology to Fragment 3 (lower case) to enable yeast recombinational cloning between the three PCR fragments.

613019 (sense):
(SEQ ID NO: 15)
agatagggttgagtgttgttccGCATTTATCAGGGTTATTGTCTCATGAG
CGG
613020 (antisense):
(SEQ ID NO: 16)
ttctacacgaaggaaagagGAGGAGAGAGTTGAACCTGGACG

Fragment 2 was amplified by PCR in a reaction composed of 10 ng of plasmid pJaL1262, 0.5 μl of PHUSION® DNA Polymerase, 20 pmol of primer 613019, 20 pmol of primer 613020, 1 μl of 10 mM dNTPs, 10 μl of 5× PHUSION® HF buffer, and 35.5 μl of water. The reaction was incubated in an EPPENDORF® MASTERCYCLER® programmed for 1 cycle at 98° C. for 30 seconds; 30 cycles each at 98° C. for 10 seconds, 60° C. for 10 seconds, and 72° C. for 2 minutes; and a 20° C. hold. The resulting 4.5 kb PCR product (Fragment 2) was used directly for yeast recombination with Fragment 1 above and Fragment 3 below.

Fragment 3 was amplified using primers 613022 (sense) and 613021 (antisense) shown below. Primer 613021 was designed to contain a flanking region of sequence homology to Fragment 2 (lower case) and primer 613022 was designed to contain a flanking region of sequence homology to Fragment 1 (lower case) to enable yeast recombinational cloning between the three PCR fragments.

613022 (sense):
(SEQ ID NO: 17)
aggttcaactctctcctcCTCTTTCCTTCGTGTAGAAGACCAGACAG
613021 (antisense):
(SEQ ID NO: 18)
tcagtgagcgaggaagcggTGTGCTGCAAGGCGATTAAGTTGG

Fragment 3 was amplified by PCR in a reaction composed of 10 ng of plasmid pEvFz1 (Example 3), 0.5 μl of PHUSION® DNA Polymerase, 20 pmol of primer 613021, 20 pmol of primer 613022, 1 μl of 10 mM dNTPs, 10 μl of 5× PHUSION® HF buffer, and 35.5 μl of water. The reaction was incubated in an EPPENDORF® MASTERCYCLER® programmed for 1 cycle at 98° C. for 30 seconds; 30 cycles each at 98° C. for 10 seconds, 60° C. for 10 seconds, and 72° C. for 2 minutes; and a 20° C. hold. The resulting 1.7 kb PCR product (Fragment 3) was used directly for yeast recombination with Fragments 1 and 2 above.

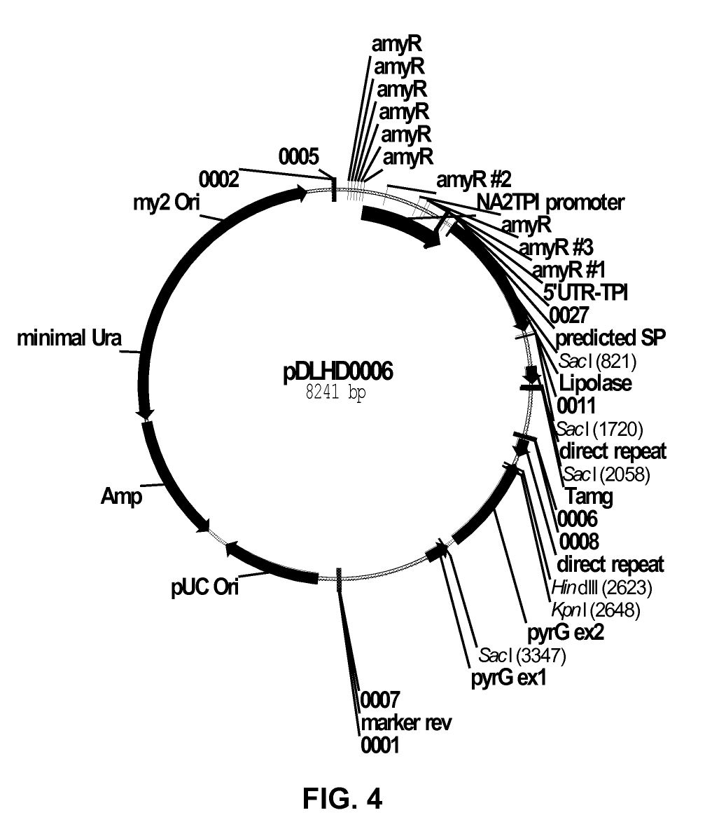

The following procedure was used to combine the three PCR fragments using yeast homology-based recombinational cloning. A 20 μl aliquot of each of the three PCR fragments was combined with 100 μg of single-stranded deoxyribonucleic acid from salmon testes (Sigma-Aldrich, St. Louis, Mo., USA), 100 μl of competent yeast cells of strain YNG318 (Saccharomyces cerevisiae ATCC 208973), and 600 μl of PLATE Buffer (Sigma Aldrich, St. Louis, Mo., USA), and mixed. The reaction was incubated at 30° C. for 30 minutes with shaking at 200 rpm. The reaction was then continued at 42° C. for 15 minutes with no shaking. The cells were pelleted by centrifugation at 5,000×g for 1 minute and the supernatant was discarded. The cell pellet was suspended in 200 μl of autoclaved water and split over two agar plates containing Synthetic defined medium lacking uridine and incubated at 30° C. for three days. The yeast colonies were isolated from the plate using 1 ml of autoclaved water. The cells were pelleted by centrifugation at 13,000×g for 30 seconds and a 100 μl aliquot of glass beads were added to the tube. The cell and bead mixture was suspended in 250 μl of P1 buffer (QIAGEN Inc., Valencia, Calif., USA) and then vortexed for 1 minute to lyse the cells. The plasmid DNA was purified using a QIAPREP® Spin Miniprep Kit. A 3 μl aliquot of the plasmid DNA was then transformed into E. coli ONE SHOT® TOP10 electrocompetent cells according the manufacturer's instructions. Fifty μl of transformed cells were spread onto LB plates supplemented with 100 μg of ampicillin per ml and incubated at 37° C. overnight. Transformants were each picked into 3 ml of LB medium supplemented with 100 μg of ampicillin per ml and grown overnight at 37° C. with shaking at 250 rpm. The plasmid DNA was purified from colonies using a QIAPREP® Spin Miniprep Kit. DNA sequencing with a 3130XL Genetic Analyzer was used to confirm the presence of each of the three fragments in a final plasmid designated pDLHD0006 (FIG. 4).

### Example 5

### Preparation of Arabidopsis thaliana Xyloglucan Endotransglycosylase 14

Arabidopsis thaliana xyloglucan endotransglycosylase (AtXET14; SEQ ID NO: 4 [native DNA sequence], SEQ ID NO: 5 [synthetic DNA sequence], and SEQ ID NO: 6 [deduced amino acid sequence]) was recombinantly produced in Aspergillus otyzae JaL355 (WO 2008/138835).

The vector pDLHD0039 was constructed to express the AtXET14 gene in multi-copy in Aspergillus oryzae. Plasmid pDLHD0039 was generated by combining two DNA fragments using restriction-free cloning: Fragment 1 containing the AtXET14 ORF and flanking sequences with homology to vector pDLHD0006 (Example 4), and Fragment 2 consisting of an inverse PCR amplicon of vector pDLHD0006.

Fragment 1 was amplified using primers AtXET14F (sense) and AtXET14R (antisense) shown below, which were designed to contain flanking regions of sequence homology to vector pDLHD0006 (lower case) for ligation-free cloning between the PCR fragments.

Primer AtXET14F (sense):
(SEQ ID NO: 19)
ttcctcaatcctctatatacacaactggccATGGCCTGTTTCGCAACCAA
ACAG
AtXET14R (antisense):
(SEQ ID NO: 20)
agctcgctagagtcgacctaGAGTTTACATTCCTTGGGGAGACCCTG

Fragment 1 was amplified by PCR in a reaction composed of 10 ng of a GENEART® vector μMA containing the AtXET14 synthetic DNA sequence cloned between the Sac I and Kpn I sites, 0.5 μl of PHUSION® DNA Polymerase (New England Biolabs, Inc., Ipswich, Mass., USA), 20 pmol of primer AtXET14F, 20 pmol of primer AtXET14R, 1 μl of 10 mM dNTPs, 10 μl of 5× PHUSION® HF buffer, and 35.5 μl of water. The reaction was incubated in an EPPENDORF® MASTERCYCLER® programmed for 1 cycle at 98° C. for 30 seconds; and 30 cycles each at 98° C. for 10 seconds, 60° C. for 10 seconds, and 72° C. for 30 seconds. The resulting 0.9 kb PCR product (Fragment 1) was treated with 1 μl of Dpn Ito remove plasmid template DNA. The Dpn I was added directly to the PCR tube, mixed well, and incubated at 3TC for 60 minutes, and then column-purified using a MINELUTE® PCR Purification Kit.

Fragment 2 was amplified using primers 614604 (sense) and 613247 (antisense) shown below.

614604 (sense):
(SEQ ID NO: 21)
taggtcgactctagcgagctcgagatc
613247 (antisense):
(SEQ ID NO: 22)
catggccagttgtgtatatagaggattgaggaaggaagag

Fragment 2 was amplified by PCR in a reaction composed of 10 ng of plasmid pDLHD0006, 0.5 μl of PHUSION® DNA Polymerase, 20 pmol of primer 614604, 20 pmol of primer 613247, 1 μl of 10 mM dNTPs, 10 μl of 5× PHUSION® HF buffer, and 35.5 μl of water. The reaction was incubated in an EPPENDORF® MASTERCYCLER® programmed for 1 cycle at 98° C. for 30 seconds; and 30 cycles each at 98° C. for 10 seconds, 60° C. for 10 seconds, and 72° C. for 4 minutes. The resulting 7.3 kb PCR product (Fragment 2) was treated with 1 μl of Dpn I to remove plasmid template DNA. Dpn I was added directly to the PCR tube, mixed well, and incubated at 3TC for 60 minutes, and then column-purified using a MINELUTE® PCR Purification Kit.

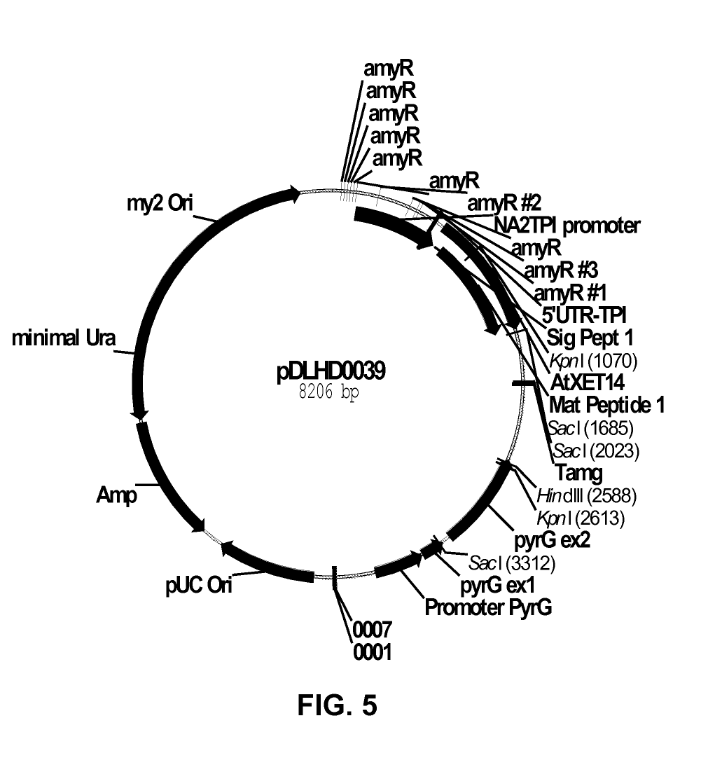

The two PCR fragments were combined using a GENEART® Seamless Cloning and Assembly Kit (Invitrogen, Carlsbad, Calif., USA) according to manufacturer's instructions. Three μl of the resulting reaction product DNA were then transformed into E. coli ONE SHOT® TOP10 electrocompetent cells. Fifty μl of transformed cells were spread onto LB plates supplemented with 100 μg of ampicillin per ml and incubated at 37° C. overnight. Individual transformant colonies were picked into 3 ml of LB medium supplemented with 100 μg of ampicillin per ml and grown overnight at 3TC with shaking at 250 rpm. The plasmid DNA was purified from colonies using a QIAPREP® Spin Miniprep Kit according to the manufacturer's instructions. DNA sequencing with a 3130XL Genetic Analyzer was used to confirm the presence of each of both fragments in the final plasmid pDLHD0039 (FIG. 5).

Aspergillus oryzae strain JaL355 was transformed with plasmid pDLHD0039 comprising the AtXET14 gene according to the following protocol. Approximately 2-5×10⁷ spores of Aspergillus otyzae JaL355 were inoculated into 100 ml of YP+2% glucose+10 mM uridine in a 500 ml shake flask and incubated at 28° C. and 110 rpm overnight. Ten ml of the overnight culture was filtered in a 125 ml sterile vacuum filter, and the mycelia were washed twice with 50 ml of 0.7 M KCl-20 mM CaCl₂. The remaining liquid was removed by vacuum filtration, leaving the mat on the filter. Mycelia were resuspended in 10 ml of 0.7 M KCl-20 mM CaCl₂ and transferred to a sterile 125 ml shake flask containing 20 mg of GLUCANEX® 200 G per ml and 0.2 mg of chitinase per ml in 10 ml of 0.7 M KCl-20 mM CaCl₂. The mixture was incubated at 37° C. and 100 rpm for 30-90 minutes until protoplasts were generated from the mycelia. The protoplast mixture was filtered through a sterile funnel lined with MIRACLOTH® into a sterile 50 ml plastic centrifuge tube to remove mycelial debris. The debris in the MIRACLOTH® was washed thoroughly with 0.7 M KCl-20 mM CaCl₂, and centrifuged at 2500 rpm (537×g) for 10 minutes at 20-23° C. The supernatant was removed and the protoplast pellet was resuspended in 20 ml of 1 M sorbitol-10 mM Tris-HCl (pH 6.5)-10 mM CaCl₂. This step was repeated twice, and the final protoplast pellet was resuspended in 1 M sorbitol-10 mM Tris-HCl (pH 6.5)-10 mM CaCl₂ to obtain a final protoplast concentration of 2×10⁷/ml.

Two micrograms of pDLHD0039 were added to the bottom of a sterile 2 ml plastic centrifuge tube. Then 100 μl of protoplasts were added to the tube followed by 300 μl of 60% PEG-4000 in 10 mM Tris-HCl (pH 6.5)-10 mM CaCl₂. The tube was mixed gently by hand and incubated at 37° C. for 30 minutes. Two ml of 1 M sorbitol-10 mM Tris-HCl (pH 6.5)-10 mM CaCl₂ were added to each transformation and the mixture was transferred onto 150 mm Minimal medium agar plates. Transformation plates were incubated at 34° C. until colonies appeared.

Thirty-five transformant colonies were picked to fresh Minimal medium agar plates and cultivated at 34° C. for four days until the strains sporulated. Fresh spores were transferred to 48-well deep-well plates containing 2 ml of YP+2% maltodextrin, covered with a breathable seal, and grown for 4 days at 28° C. with no shaking. After 4 days growth the culture medium was assayed for xyloglucan endotransglycosylase activity and for xyloglucan endotransglycosylase expression by SDS-PAGE.

Xyloglucan endotransglycosylase activity was measured using the iodine stain assay described in Example 1. The assay demonstrated the presence of xyloglucan endotransglycosylase activity in several transformants.

SDS-PAGE was performed as described in Example 1. SDS-PAGE analysis indicated that several transformants expressed a protein of approximately 32 kDa corresponding to AtXET14.

### Example 6

### Generation of Fluorescein Isothiocyanate-Labeled Xyloglucan

Fluorescein isothiocyanate-labeled xyloglucan oligomers (FITC-XGOs) were generated by reductive amination of the reducing ends of xyloglucan oligomers (XGOs) according to the procedure described by Zhou et al., 2006, Biocatalysis and Biotransformation 24: 107-120), followed by conjugation of the amino groups of the XGOs to fluorescein isothiocyanate isomer I (Sigma Aldrich, St. Louis, Mo., USA) in 100 mM sodium bicarbonate pH 9.0 for 24 hours at room temperature. Conjugation reaction products were concentrated to dryness in vacuo, dissolved in 0.5 ml of deionized water, and purified by silica gel chromatography, eluting with a gradient from 100:0:0.04 to 70:30:1 acetonitrile:water:acetic acid as mobile phase. Purity and product identity were confirmed by evaporating the buffer, dissolving in D₂O (Sigma Aldrich, St. Louis, Mo., USA), and analysis by ¹H NMR using a Varian 400 MHz MercuryVx (Agilent, Santa Clara, Calif., USA). Dried FITC-XGOs were stored at 20° C. in the dark, and were desiccated during thaw.

Twenty-four ml of 10 mg of tamarind seed xyloglucan (Megazyme, Bray, UK) per ml of deionized water, 217 μl of 7.9 mg of FITC-XGOs per ml of deionized water, 1.2 ml of 400 mM sodium citrate pH 5.5, and 600 μl of 1.4 mg of VaXET16 per ml of 20 mM sodium citrate pH 5.5 were mixed thoroughly and incubated overnight at room temperature. Following overnight incubation, FITC-XG was precipitated by addition of ice cold ethanol to a final volume of 110 ml, mixed thoroughly, and incubated at 4° C. overnight. The precipitated FITC-XG was washed with water and then transferred to Erlenmeyer bulbs. Residual water and ethanol were removed by evaporation using an EZ-2 Elite evaporator (SP Scientific/Genevac, Stone Ridge, N.Y., USA) for 4 hours. Dried samples were dissolved in water, and the volume was adjusted to 48 ml with deionized water to generate a final FITC-XG concentration of 5 mg per ml with an expected average molecular weight of 100 kDa.

### Example 7

### Fluorescence Polarization Assay for Xyloglucan Endotransglycosylation activity

Xyloglucan endotransglycosylation activity was assessed using the following assay. Reactions of 200 μl containing 1 mg of tamarind seed xyloglucan per ml, 0.01 mg/ml FITC-XGOs prepared as described in Example 6, and 10 μl of appropriately diluted XET were incubated for 10 minutes at 25° C. in 20 mM sodium citrate pH 5.5 in opaque 96-well microtiter plates. Fluorescence polarization was monitored continuously over this time period, using a SPECTRAMAX® M5 microplate reader (Molecular Devices, Sunnyvale, Calif., USA) in top-read orientation with an excitation wavelength of 490 nm, an emission wavelength of 520 nm, a 495 cutoff filter in the excitation path, high precision (100 reads), and medium photomultiplier tube sensitivity. XET-dependent incorporation of fluorescent XGOs into non-fluorescent xyloglucan (XG) results in increasing fluorescence polarization over time. The slope of the linear regions of the polarization time progress curves was used to determine the activity.

### Example 8

### Purification of Vigna angularis Xyloglucan Endotransglycosylase 16

One liter solutions of crude fermentation broth of Vigna angularis were filtered using a 0.22 μm STERICUP® filter (Millipore, Bedford, Mass., USA) and the filtrates were stored at 4° C. Cell debris was resuspended in 1 liter of 0.25% TRITON® X-100 (4-(1,1,3,3-tetramethylbutyl)phenyl-polyethylene glycol; Sigma Aldrich, St. Louis, Mo., USA)-20 mM sodium citrate pH 5.5, incubated at least 30 minutes at room temperature, and then filtered using a 0.22 μm STERICUP® filter. The filtrates containing Vigna angularis xyloglucan endotransglycosylase 16 (VaXET16) were pooled and concentrated to a volume between 500 and 1500 ml using a VIVAFLOW® 200 tangential flow concentrator (Millipore, Bedford, Mass., USA) equipped with a 10 kDa molecular weight cutoff membrane.

The concentrated filtrates were loaded onto a 150 ml Q SEPHAROSE® Big Beads column (GE Healthcare Lifesciences, Piscataway, N.J., USA), pre-equilibrated with 20 mM sodium citrate pH 5.5, and eluted isocratically with the same buffer. The eluent was loaded onto a 75 ml Phenyl SEPHAROSE® HP column (GE Healthcare Lifesciences, Piscataway, N.J., USA) pre-equilibrated in 20% ethylene glycol-20 mM sodium citrate pH 5.5. VaXET16 was eluted using a linear gradient from 20% to 50% of 70% ethylene glycol in 20 mM sodium citrate pH 5.5 over 4 column volumes.

Purified VaXET16 was quantified using a BCA assay (Pierce, Rockford, Ill., USA) in a 96-well plate format with bovine serum albumin (Pierce, Rockford, Ill., USA) as a protein standard at concentrations between 0 and 2 mg/ml and was determined to be 1.40 mg/ml. VaXET16 homogeneity was confirmed by the presence of a single band at approximately 32 kDa using a β-16% gradient CRITERION® Stain Free SDS-PAGE gel, and imaging the gel with a Stain Free Imager using the following settings: 5-minute activation, automatic imaging exposure (intense bands), highlight saturated pixels=ON, color=Coomassie, and band detection, molecular weight analysis and reporting disabled.

The activity of the purified VaXET16 was determined by measuring the rate of incorporation of fluorescein isothiocyanate-labeled xyloglucan oligomers into tamarind seed xyloglucan (Megazyme, Bray, UK) by fluorescence polarization (as described in Example 7). The apparent activity was 18.5±1.2 P s⁻¹ mg⁻¹.

The purified VaXET16 preparation was tested for background enzyme activities including xylanase, amylase, cellulase, beta-glucosidase, protease, amyloglucosidase, and lipase using standard assays as shown below.

Xylanase activity was assayed using wheat arabinoxylan as substrate at pH 6.0 and 50° C. Xylan hydrolysis was assessed colorimetrically at 405 nm by addition of alkaline solution containing PHBAH. One FXU(S) is defined as the endoxylanase activity using Shearzyme® (Novozymes A/S) as a standard.

Amylase activity was assayed using starch as substrate at pH 2.5 and 37° C. Starch hydrolysis was assessed by measuring the residual starch colorimetrically at 600 nm by addition of iodine solution. One FAU(A) is defined as the acid alpha-amylase activity using acid fungal alpha-amylase (available from Novozymes A/S) as a standard.

Amylase activity was assayed using (4,6-ethylidene(G7)-p-nitrophenyl(G1)-α,D-maltoheptaoside (4,6-ethylidene-G7-pNP) as substrate at pH 7 and 37° C. Hydrolysis of the substrate produces p-nitrophenol, and was assessed colorimetrically at 405 nm. One FAU(F) is defined as fungal alpha-amylase units using Fungamyl® (Novozymes A/S) as a standard.

Cellulase activity was assayed using carboxymethylcellulose (CMC) as substrate at pH 5.0 and 50° C. CMC hydrolysis was assessed colorimetrically at 405 nm by addition of an alkaline solution containing para-hydroxybenzoic acid hythazide (PHBAH). One CNU(B) is defined as the cellulase activity using NS22084 enzyme (Novozymes A/S) as a standard.

Beta-glucosidase activity was assayed using cellobiose as substrate at pH 5.0 and 50° C. Production of glucose from cellobiose was assessed using a coupled enzyme assay with hexokinase and glucose-6-phosphate dehydrogenase converting glucose to 6-phosphoglucanate following reduction of NAD to NADH at 340 nm. One CBU(B) is defined as the amount of enzyme which releases 2 μmole of glucose per minute using cellobiase as a standard.

The protease assay was performed using an EnzChek® Protease Assay Kit (green fluorescence) (Life Technologies, Inc., Grand Island, N.Y., USA) with casein as substrate at pH 6 or 9 and ambient temperature. One KMTU is defined as a kilo microbial trypsin unit related to the amount of enzyme that produces 1 μmole of p-nitroaniline per minute.

Amyloglucosidase activity was assayed using maltose as substrate at pH 4.3 and 37° C. Conversion of maltose to glucose was assessed using a coupled enzyme assay with hexokinase and glucose-6-phosphate dehydrogenase converting glucose to 6-phosphoglucanate following reduction of NAD to NADH at 340 nm. One AGU is defined as amyloglucosidase units using AMG® (Novozymes A/S) as a standard.

The 4-methylumbelliferyl beta-D-lactoside (MUL) assay was performed at pH 7 and ambient temperature and measured fluorometrically at 360 nm excitation and 465 nm emission.

Lipase activity was assayed using 4-nitropenyl butyrate (pNP-butyrate) as substrate at pH 7.5 and ambient temperature. pNP-butyrate hydrolysis was assessed colorimetrically following p-nitrophenol release at 405 nm. One LU is defined as the amount of enzyme which releases 1 μmole of titratable butyric acid using LIPOLASE® (Novozymes A/S) as a standard.

| | | Additional | | |
|---|---|---|---|---|
| | | Assay | Activity | Activity |
| Assay | Substrate | Dilution | Units | Units/ml |
| Xylanase FXU(S) | Wheat | 4-fold | FXU(S) | ND |
| | arabinoxylan | | | |
| Amylase FAU(A) | Starch | 4-fold | FAU(A) | ND |
| Amylase FAU(F) | Ethylidene- | 4-fold | FAU(F) | ND |
| | G7-pNp | | | |
| Cellulase CNU(B) | CMC | 4-fold | CNU(B) | ND |
| Beta-glucosidase | Cellobiose | 4-fold | CBU(B) | ND |
| CBU(B) | | | | |
| Protease, pH 6 | Casein | none | KMTU | 740 |
| (EnzCheck) | | | | |
| Protease, pH 9 | Casein | none | KMTU | 332 |
| (EnzCheck) | | | | |
| Amyloglucosidase | Maltose | 4-fold | AGU | ND |
| AGU | | | | |
| MUL | MUL | none | Unitless | ND |
| Lipase | pNP-Butyrate | none | LU | 0.02 |

### Example 9

### Purification of Arabidopsis thaliana Xyloglucan Endotransglycosylase 14

The purification and quantification of the Arabidopsis thaliana xyloglucan endotransglycosylase 14 (AtXET14) was performed as described for VaXET16 in Example 8, except that elution from the Phenyl SEPHAROSE® HP column was performed using a linear gradient from 40% to 90% of 70% ethylene glycol in 20 mM sodium citrate pH 5.5 over 4 column volumes.

AtXET14 homogeneity was confirmed by the presence of a single band at approximately 32 kDa using a 8-16% CRITERION® Stain Free SDS-PAGE gel, and imaging the gel with a Stain Free Imager using the following settings: 5-minute activation, automatic imaging exposure (intense bands), highlight saturated pixels=ON, color=Coomassie, and band detection, molecular weight analysis and reporting disabled.

Purified AtXET14 was quantified using a BCA assay in a 96-well plate format with bovine serum albumin as a protein standard at concentrations between 0 and 2 mg/ml and was determined to be 1.49 mg/ml.

The activity of the purified AtXET14 was determined as described in Example 7. The apparent activity was 34.7±0.9 P s⁻¹ mg⁻¹.

The purified AtXET14 preparation was tested for background activities including xylanase, amylase, cellulase, beta-glucosidase, protease, amyloglucosidase, and lipase using standard assays as shown below. The standard assays are described in Example 8.

| | | Additional | | |
|---|---|---|---|---|
| | | Assay | Activity | Activity |
| Assay | Substrate | Dilution | Units | Units/ml |
| Xylanase FXU(S) | Wheat | 4-fold | FXU(S) | ND |
| | arabinoxylan | | | |
| Amylase FAU(A) | Starch | 4-fold | FAU(A) | ND |
| Amylase FAU(F) | Ethyliden- | 4-fold | FAU(F) | ND |
| | G7-pNp | | | |
| Cellulase CNU(B) | CMC | 4-fold | CNU(B) | ND |
| Beta-glucosidase | Cellobiose | 4-fold | CBU(B) | ND |
| CBU(B) | | | | |
| Protease, pH 6 | Casein | none | KMTU | 82 |
| (EnzCheck) | | | | |
| Protease, pH 9 | Casein | none | KMTU | 53 |
| (EnzCheck) | | | | |
| Amyloglucosidase | Maltose | 4-fold | AGU | ND |
| AGU | | | | |
| MUL | MUL | none | Unitless | ND |
| Lipase | pNP-Butyrate | none | LU | 0.24 |

### Example 10

### Enhancement of Binding of Fluorescein Isothiocyanate-Labeled Xyloglucan to Cellulose by Vigna angularis Xyloglucan Endotransglycosylase 16

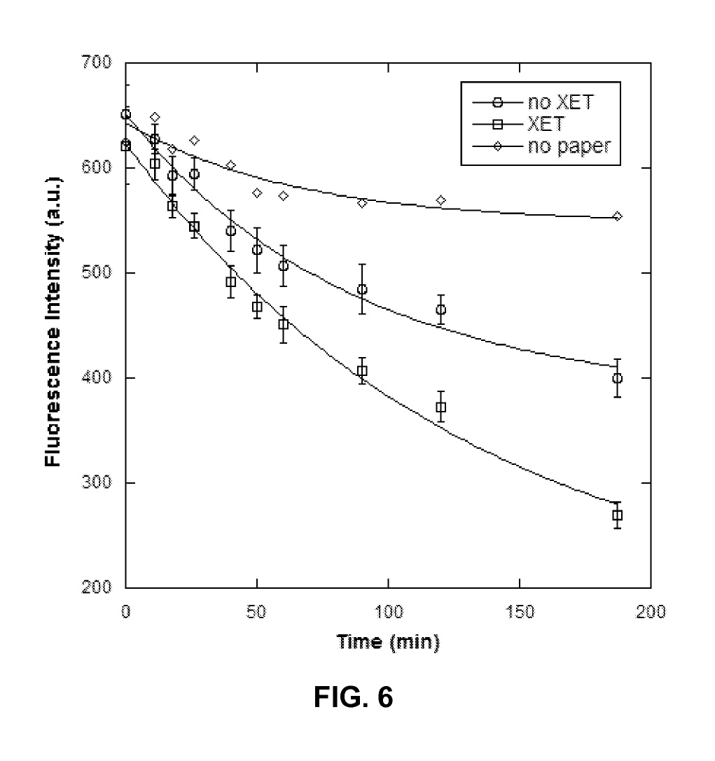

Binding of fluorescein isothiocyanate-labeled xyloglucan (FITC-XG) to filter paper was assessed according to the following protocol. Circular cuttings of Whatman #1 filter paper were generated using a 0.5 inch diameter circular paper punch. Six replicate cuttings of the filter paper were each covered with a 2 ml volume of 317 nM FITC-XG, as assessed by absorbance at 488 nm, with or without 2.2 μM VaXET16 in Costar #3513, 12-well cell culture cluster plates (Corning, Tewksbury, Mass., USA). Solutions were mixed by pipetting up and down over the surface of the filter paper, and then the plates were incubated at room temperature with gentle shaking on a rocking platform (VWR, Radnor, Pa., USA) at 3 rpm for up to 3 hours. Negative control incubations on each plate contained no paper. At the times indicated in FIG. 6, 1 ml aliquots of the solution phase were removed, the fluorescence intensities were measured, and then the aliquots were returned to the incubation well. Fluorescence intensities were measured in 1 ml disposable cuvettes using a SPECTRAMAX® M5 spectrophotometer (Molecular Devices, Sunnyvale, Calif., USA) with the following optical parameters: λₑₓ=490 nm, λₑₘ, =520 nm, using a 495 nm cut-off filter in the excitation path, photomultiplier tube sensitivity was high, and maximum number of sample reads (100) for precision. Intensities were plotted as the mean and standard deviation of the replicate samples.

FIG. 6 shows the fluorescence intensity of the solution phase of FITC-XG incubated with filter paper, incubated with filter paper in the presence of VaXET16, or incubated with no filter paper. The fluorescence intensity of the solution decreased with time for all samples. The control incubation that contained no filter paper showed a small loss of fluorescence (<15%) likely due to adsorption of the FITC-XG to the culture plate walls and/or due to photobleaching of the fluorescein. The incubation of FITC-XG and filter paper without VaXET16 showed a 38% loss of intensity in 3 hours. The incubation of FITC-XG and filter paper with VaXET16 showed a 55% loss of intensity over the same 3 hour incubation time. Data were fit with a single exponential. The intensities at equilibrium determined from the data fit were 547±18.9, 380±25.5, and 170±53 for FITC-XG incubated with no filter paper, with filter paper, and with filter paper and VaXET16, respectively.

### Example 11

### Assessment of Fluorescein Isothiocyanate-Labeled Xyloglucan Binding to Damaged and Undamaged Raspberry Leaves

Binding of FITC-XG to plant leaves was assessed in the following manner. A 2.5 cm×2.75 cm rectangular hole was cut in a piece of ⅛-inch acrylic sheet as a template to ensure leaf pieces were of equivalent size.

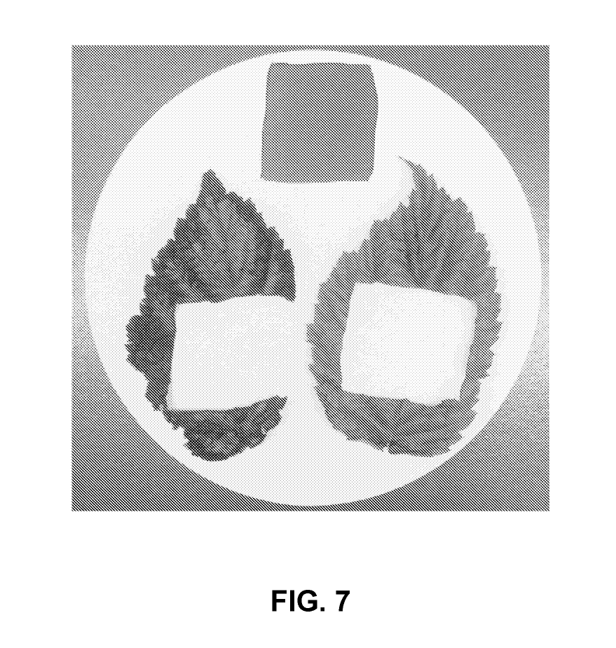

Leaf pieces were visually assessed for damage, and pristine leaves were considered undamaged, while leaves showing approximately equivalent amounts of wilting/browning were considered damaged. Rectangular pieces of each set of leaves, damaged and undamaged, were generated by cutting leaves with a razor blade, using the acrylic sheet as a template; a representative example is demonstrated in FIG. 7. An equivalently treated piece of Whatman #1 filter paper was used as a positive control. A 100 μl volume of 1.27 μM FITC-XG stock solution was diluted in 2 ml of water in a 1 cm, reduced volume disposable plastic cuvette to generate a final concentration of 63.5 nM FITC-XG. A square piece of undamaged or damaged raspberry leaf, or filter paper, was added by rolling and folding the material into the top of the cuvette. The cuvette was inspected to ensure that the solid material did not extend into the optical path of the cuvette, and that the FITC-XG solution covered the material. Samples were incubated for 2 hours, then the fluorescence spectrum of each cuvette was recorded using a SPECTRAMAX® M5 spectrophotometer with λₑₓ=490 nm, λₑₘ=500-650 nm, a 495 nm cut-off filter in the excitation path, PMT sensitivity=high, and maximum reps for precision. The damaged and undamaged leaves had 74% and 75% intensity, respectively, and the filter paper had 82% intensity of a control incubation containing no cellulosic material, indicating that the FITC-XG had bound to the leaves and paper.

To better account for effects of mixing, a further set of experiments was performed with squares of damaged or undamaged leaves by incubating them in a Corning Costar #3516 6-well cell culture cluster flat-bottom plates with lid (Corning, Tewksbury, Mass., USA) with shaking on a rocking platform (VWR, Radnor, Pa., USA) at 3 rpm for 22 hours. The leaves were incubated in 2 ml of 317 nM FITC-XG (fluorophore concentration) or in an equivalent volume of water containing no FITC-XG. A similar incubation of FITC-XG was performed in the absence of a leaf piece. In the culture plates, the leaves fit into wells without folding and rolling. At the indicated times, 100 μl of each sample was removed, the fluorescence intensity was measured in Costar 96-well flat bottom plates (λₑₓ=490 nm, λₑₘ=520 nm, 495 nm cut-off filter, maximum precision, medium PMT sensitivity), and the aliquots were returned to the leaf incubation. To confirm that loss of fluorescence intensity in the presence of the damaged leaf was not due to quenching by soluble material released by the leaf, after 5 hours of incubation, 100 μl of the FITC-XG incubated without a leaf piece was mixed with increasing volumes of an extract from a damaged leaf in water and minimal change in fluorescence was observed. After 22 hours of incubation, 100 μl aliquots were removed and the spectra of each sample were recorded using the optical arrangement described above.

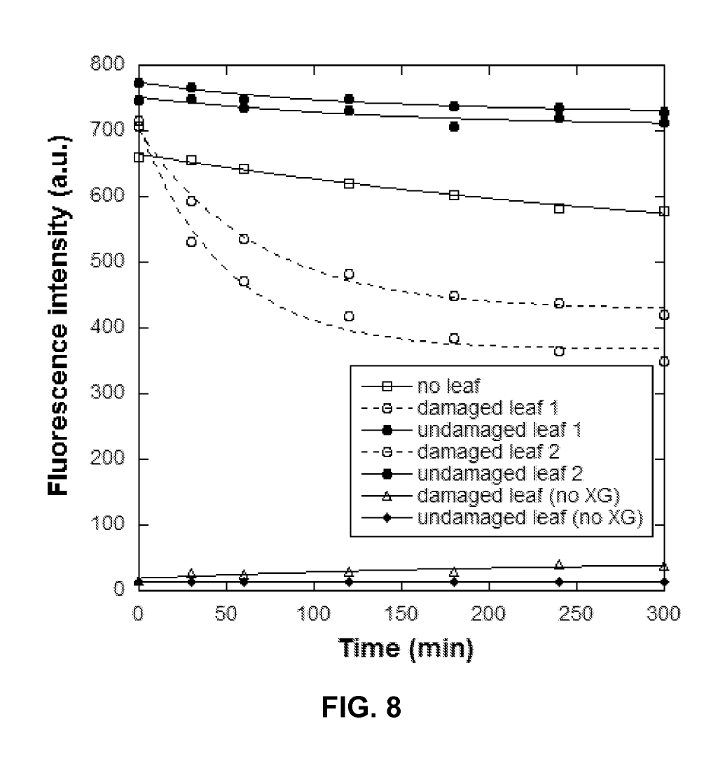

FIG. 8 shows the fluorescence intensity of each supernatant from the leaf incubations at various times. From the decrease in fluorescence intensity of samples containing leaf, it was evident that FITC-XG was binding to the leaf pieces. The damaged leaf pieces bound significantly more FITC-XG than the undamaged pieces at the incubation times examined.

### Example 12

### pH Dependence of the Enhancement of Binding of Fluorescein Isothiocyanate-Labeled Xyloglucan to Cellulose by Vigna angularis Xyloglucan Endotransglycosylase 16 (VaXET16)

The pH dependence of FITC-XG binding to filter paper was assessed according to the following protocol. Whatman #1 filter paper was cut into circular discs using a standard paper punch, approximately 0.7 cm in diameter. Binding reactions of 200 μl containing 16 mg of Whatman #1 filter paper per ml and various concentrations of FITC-XG (0.05, 0.1, 0.2, 0.5, 1, 2, 2.5, and 4.5 mg per ml) in 50 mM Britton-Robinson buffer (1:1:1 boric acid, phosphoric acid, and acetic acid) were performed in 500 μl Nunc U96 PP polypropylene 96-well plates (Thermo Scientific, Waltham, Mass., USA). The binding reactions contained either 1 μM VaXET16 or no VaXET16. Plates were sealed using an ALPS 3000 plate sealer (Thermo Scientific, Waltham, Mass., USA), and incubated vertically at 25° C. in an IN NOVA® 40 shaker incubator (New Brunswick Scientific, Enfield, Conn., USA) with shaking at 150 rpm. After 36 hours of incubation, residual fluorescence of the supernatant was measured as described in Example 11, except that 200 μl aliquots were measured in Costar 9017 flat bottomed microtiter plate (Corning, Tewksbury, Mass., USA). Residual fluorescence is reported relative to a control experiment that contained no filter paper.

Data were fit to a binding isotherm, and apparent binding capacities were determined.

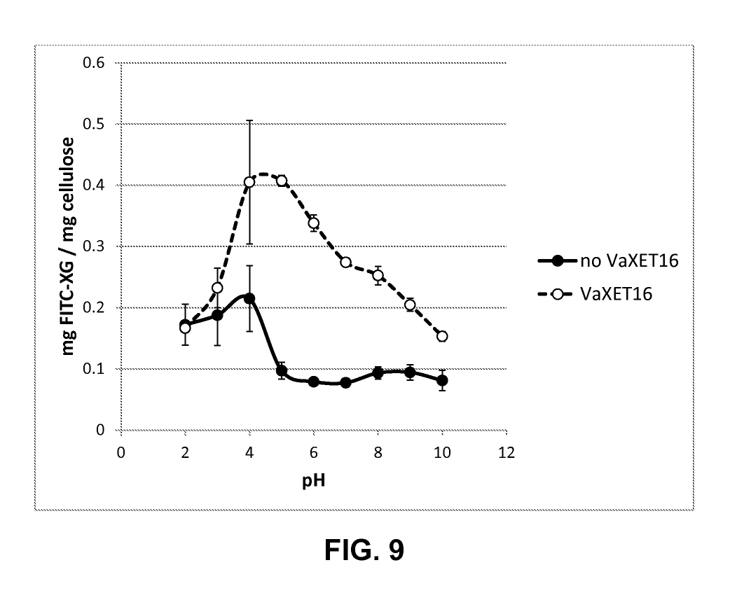

FIG. 9 shows the binding capacity of cellulose for FITC-XG at various pH values in the presence and absence of VaXET16.

At pH values where VaXET16 is active (pH 4-8), the binding capacity of cellulose for xyloglucan was 2-4 fold greater when VaXET16 was present than when VaXET16 was absent. At pH values below the active pH of VaXET16 (pH 2-3), there was no difference in binding capacity and at pH values where VaXET16 is less active (pH β-10) the enhancement in binding capacity was reduced. These data indicate that the activity of VaXET16 was responsible for binding enhancement.

### Example 13

### Temperature-Dependence of the Enhancement of Binding of Fluorescein Isothiocyanate-Labeled Xyloglucan to Cellulose by Vigna angularis Xyloglucan Endotransglycosylase 16 (VaXET16)

The temperature dependence of binding of FITC-XG to filter paper was assessed as described in Example 10, with the following exceptions. 500 μl binding reactions were performed in 1.1 ml 96-deep well plates (Axygen, Union City, Calif., USA) at 4, 25, 37 and 50° C. using 10 mg of Whatman #1 filter paper per ml, with or without 1 μM VaXET16 in 50 mM sodium citrate pH 5.5.

Plates were sealed using an ALPS 3000 plate sealer, mixed thoroughly by shaking the sealed plate, and then incubated at the indicated temperatures as described in Example 10 for 38 hours with shaking at 150 rpm. Fluorescence of the supernatant fractions was measured as described in Example 10.

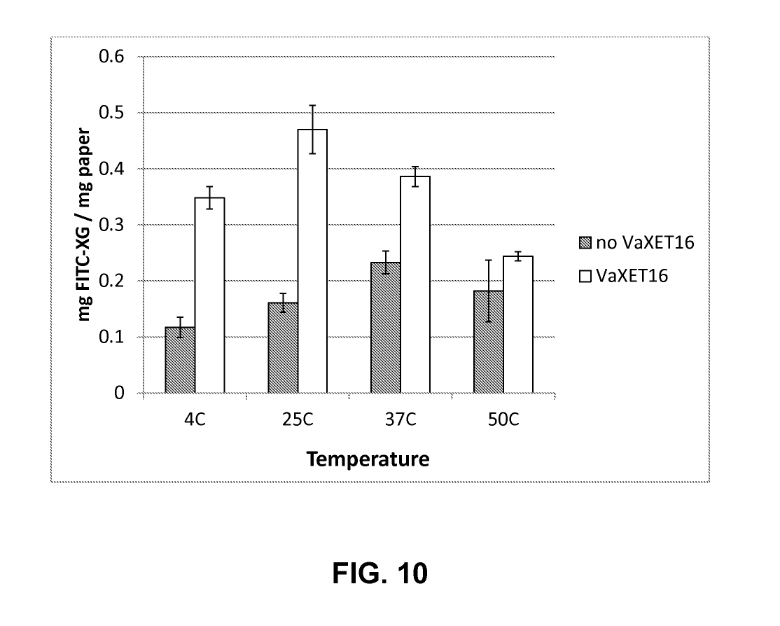

FIG. 10 shows the binding capacity of cellulose for FITC-XG at various temperatures in the presence and absence of VaXET16.

At all temperatures below 50° C., more FITC-XG bound to the cellulose in the presence of VaXET16 compared to without VaXET16. At 50° C., near the melting temperature of the enzyme, marginal to no enhancement was observed in the presence of VaXET16.

### Example 14

### Enhancement of Binding of Fluorescein Isothiocyanate-Labeled Xyloglucan to Rose Leaf Cuttings by Vigna angularis Xyloglucan Endotransglycosylase 16 (VaXET16)

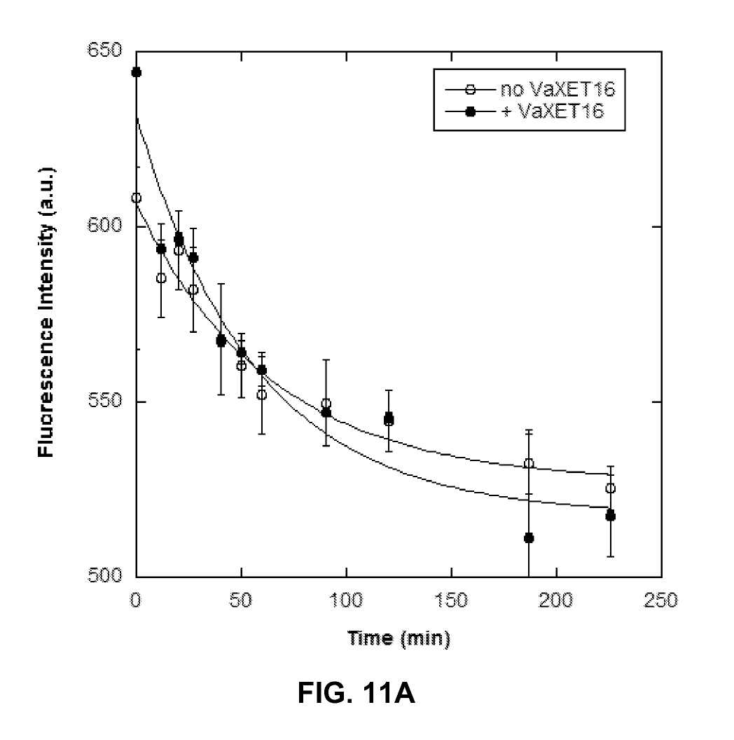

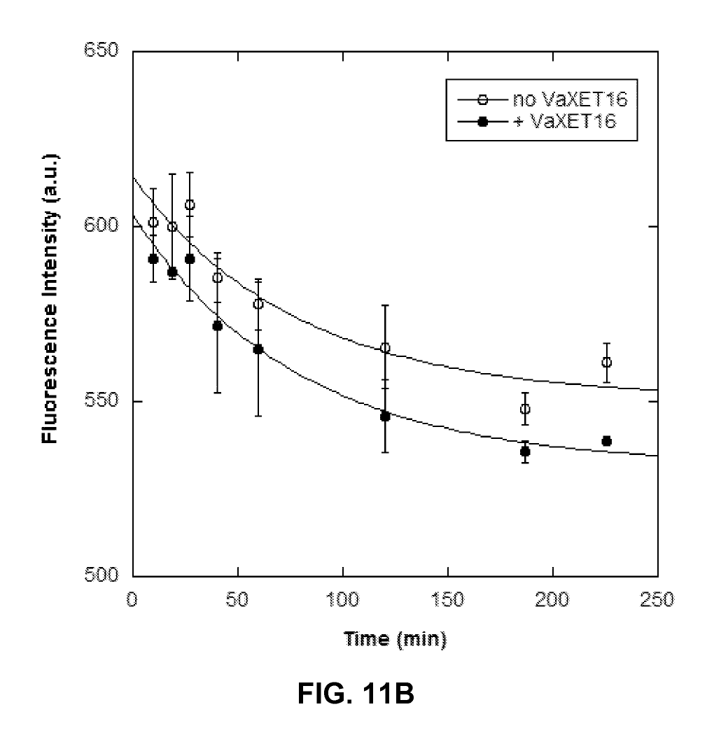

Binding of FITC-XG to leaves was assessed as described in Example 7 with the following exceptions. Circular cuttings of both damaged rose leaves and undamaged rose leaves, leaves that had visually apparent brown damaged patches, or had no visually apparent brown patches, respectively, were generated using a 0.5 inch diameter circular paper punch. Damaged leaf cuttings were visually assessed, and as much as possible contained a similar degree of brown surface area. Six replicate cuttings of each substrate were covered with a 2 ml volume of 317 nM FITC-XG with or without 7.8 mg of VaXET16 per ml of 20 mM sodium citrate pH 5.5 in Costar #3513, 12-well cell culture cluster plates. Solutions were mixed by pipetting up and down over the surface of the leaf cutting, and then incubated at room temperature with shaking for 3 hours. Negative control incubations on each plate contained no leaf cutting. At the times indicated in FIGS. 11A and 11B, 1 ml aliquots were removed, the fluorescence intensities were measured as described in Example 7, and the aliquots were returned to the incubation well. The measured fluorescence intensities were grouped into 2 statistically different populations as determined by Student's T-test, for both the damaged and undamaged leaf samples. The change in the fluorescence intensity of the higher intensity population was observed to be altered by VaXET16, generating a larger change in fluorescence, hence a greater extent of binding.

FIG. 11A shows the fluorescence intensity of the solution phase of the undamaged rose leaf cuttings incubated with FITC-XG with or without VaXET16 as a function of incubation time.

FIG. 11B shows the fluorescence intensity of the solution phase of the damaged rose leaf cuttings incubated with FITC-XG with or without VaXET16 as a function of incubation time.

Exponential fits of the intensity decays indicated a greater total change in fluorescence intensity for samples incubated with VaXET16. The total change in fluorescence for the undamaged leaf incubation was 79.6±5.6 with no VaXET16 and 114.1±10.0 with VaXET16. The total change in fluorescence for the damaged leaf was 63.5±9.0 with no VaXET16 and 71.6±5.7 with VaXET16. These results indicate that more of the FITC-XG bound to the leaf cuttings in the presence of VaXET16.

### Example 15

### Fluorescein Isothiocyanate-Labeled Xyloglucan Confirms Association of Xyloglucan with Roots and Seeds

To confirm that xyloglucan can associate with roots and seeds in addition to leaves, 1 mg per ml of FITC-XG with or without 1 μM VaXET16 in 40 mM sodium citrate pH 5.5 were applied to strawberry roots or tomato seeds. In 500 μl reactions, the samples were incubated at room temperature for 24 hours on a rocking platform (VWR, Radnor, Pa., USA) in 48 well culture plates with lids (Corning, Inc., Corning, N.Y., USA).

Thin sections of each sample were cut using a razor blade and laid onto a FisherFinest Premium 3″×1″×1 mm microscope slide (Fisher Scientific, Inc., Pittsburgh, Pa., USA). Approximately 20 μl of deionized water were applied to the slide around the sample and the sample was covered with a Fisherbrand 22×22-1.5 microscope coverslip (Fisher Scientific, Inc., Pittsburgh, Pa., USA) before sealing the coverslip to the slide using nail polish.

Laser scanning confocal microscopy was performed using an Olympus FV1000 laser scanning confocal microscope (Olympus, Center Valley, Pa., USA). Data were acquired utilizing the 488 nm line of an argon ion laser excitation source with either a 10× air gap or a 40× oil immersion objective lens as indicated. All images were obtained using the same excitation intensity and PMT voltage; hence relative fluorescence intensities were comparable between images.

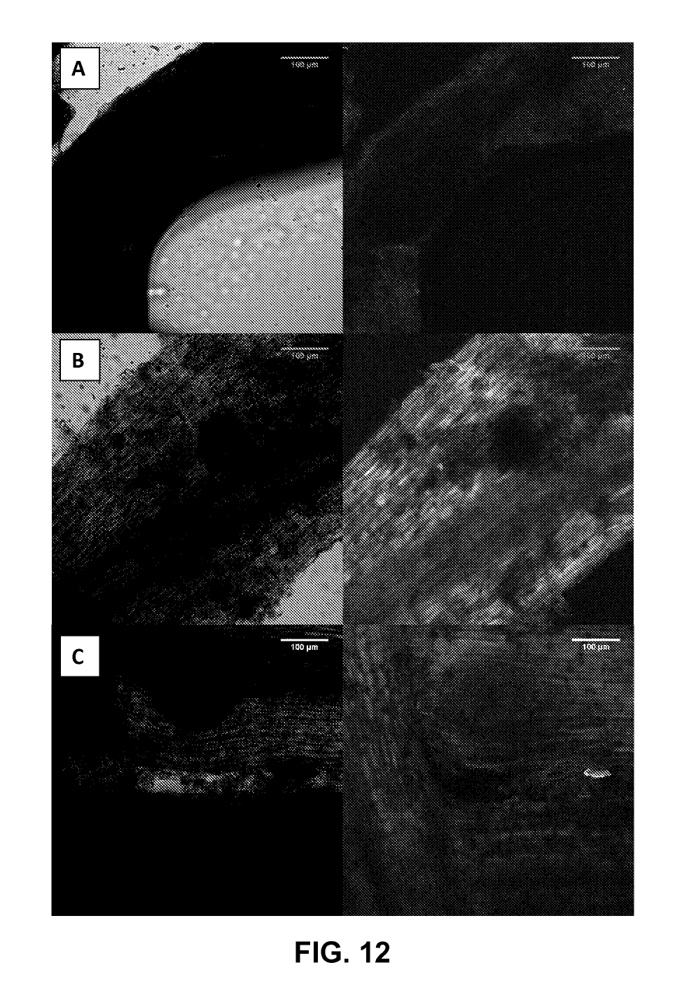

FIGS. 12 to 15 show laser scanning confocal microscope images that compare roots or seeds incubated with (panel A) sodium citrate pH 5.5, (panel B) FITC-XG in sodium citrate pH 5.5, and (panel C) FITC-XG with VaXET16 in sodium citrate pH 5.5.

FIG. 12 shows laser scanning confocal microscope images of strawberry roots using a 10× objective lens.

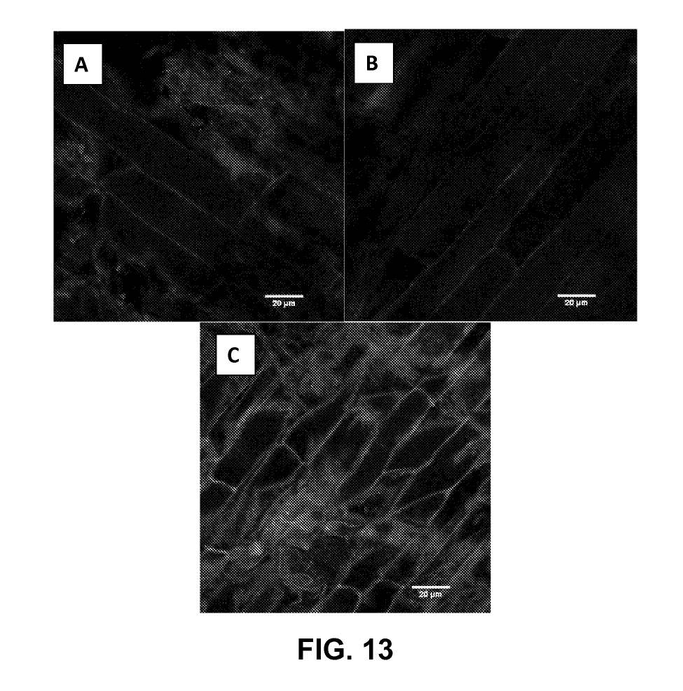

FIG. 13 shows laser scanning confocal microscope images of strawberry roots using a 40× objective lens.

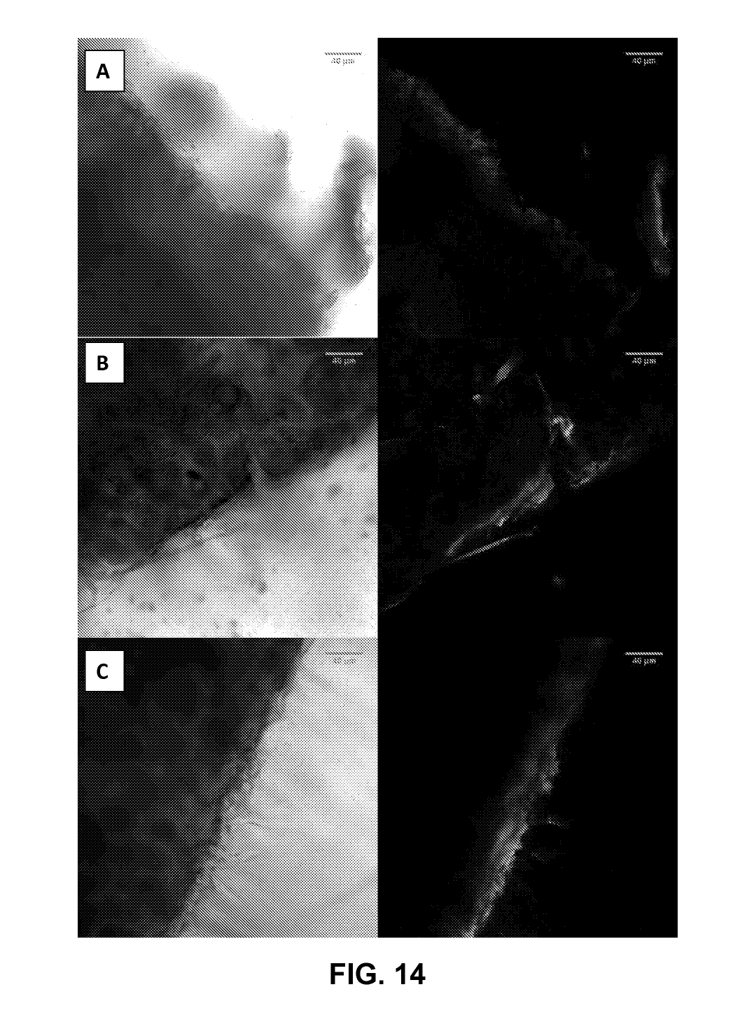

FIG. 14 shows laser scanning confocal microscope images of tomato seed edges using a 40× objective lens.

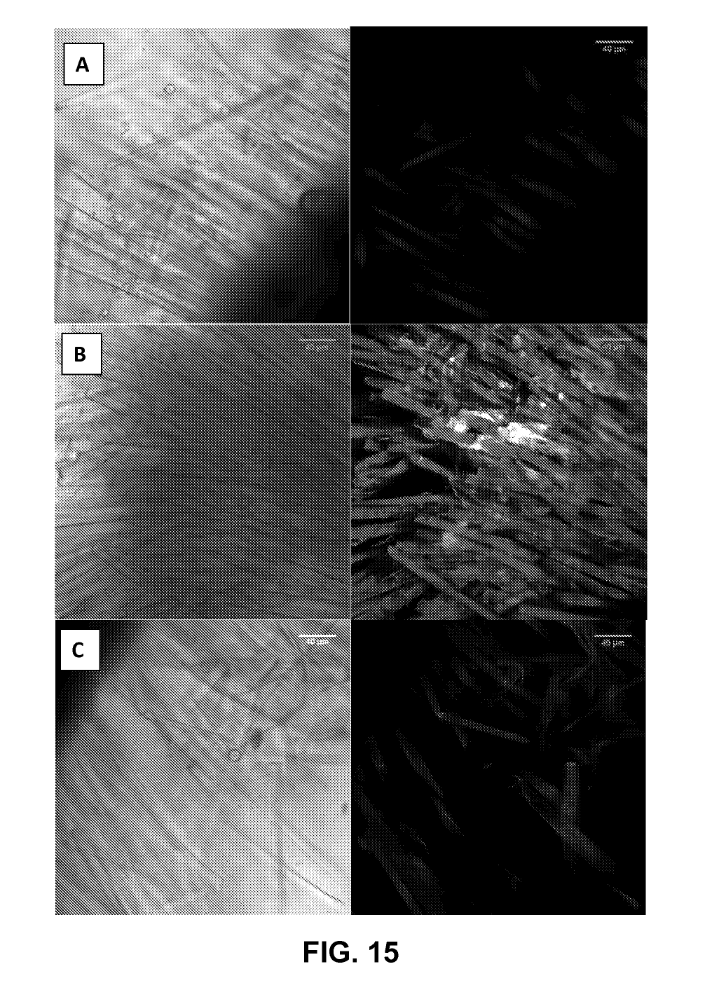

FIG. 15 shows laser scanning confocal microscope images of tomato seed hairs using a 40× objective lens.

In each case the confocal microscopy image indicates that the fluorescein isothiocyanate-labeled xyloglucan associated with the roots or seeds in the presence or absence of VaXET.

### Example 16

### Xyloglucan and Vigna angularis XET16 Enhance Binding of Biopesticide to Plant Leaves

To illustrate the use of polymeric xyloglucan and VaXET16 to enhance the binding of natural or biological pesticides to plant material and hence to plants, TAEGRO® (Novozymes, A/S, Bagsvaerd, Denmark) was assayed for binding to Whatman #1 filter paper or raspberry leaves cut with a circular paper punch as previously described (Example 14).

A 10 mg/ml slurry of TAEGRO® in deionized water was generated. Fifty μl of the TAEGRO® slurry was incubated overnight at ambient conditions in 20 mM sodium citrate pH 5.5 with or without 1 mg/ml tamarind seed xyloglucan (Megazyme, Bray, UK) with or without 0.5 mg/ml microcrystalline cellulose (AVICEL®) with or without 0.56 μM VaXET16 and a circular disc cutting of a raspberry leaf or filter paper, in 1 ml total volume in Costar #3513 12-well culture plates with shaking. Periodically, the suspensions were pipetted up and down, and the thoroughly mixed suspension was pipetted over each leaf or filter paper disc to ensure full coverage. After 24 hours, 1 ml of deionized water was added to each well, and the leaf or filter paper cuttings were removed from the suspension. The remaining TAEGRO® suspensions in each well were transferred to disposable cuvettes and the absorbance of each suspension was measured at 600 nm.

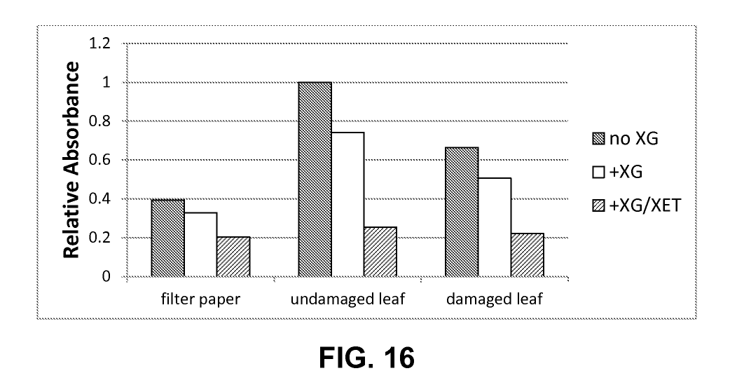

FIG. 16 shows the relative optical density of the unbound TAEGRO® suspensions at 600 nm. In each sample, the presence of polymeric xyloglucan (XG) reduced the A₆₀₀ relative to the sample containing no polymeric xyloglucan. The combination of xyloglucan and VaXET16 reduced the A₆₀₀ relative to the presence of xyloglucan alone. These data indicate that polymeric xyloglucan, and particularly polymeric xyloglucan with VaXET16, can enhance the association of TAEGRO® with leaves and with cellulose. Comparing the cellulosic materials, the undamaged leaves had the highest A₆₀₀, and the damaged leaves had a lower A₆₀₀. The lowest overall A₆₀₀ values were observed for the filter paper incubated samples. However, in the presence of polymeric xyloglucan and VaXET16, the A₆₀₀ approached a similar, low value for all the materials tested.

### Example 17

### Construction of Sub-Cloning Plasmid pBM324, Containing the Red Fluorescent Protein dsRed Gene Under Transcriptional Control of the Triple Promoter

Plasmid pBM324 was constructed as a subcloning plasmid containing the red fluorescent protein dsRed gene (SEQ ID NO: 23 [DNA sequence] and SEQ ID NO: 24 [amino acid sequence]) under transcriptional control of a triple promoter composed of the Bacillus licheniformis amyL promoter, short consensus B. amyloliqufaciens amyQ promoter, and B. thuringiensis cryIIIA promoter (U.S. Pat. No. 8,268,586).

The following primers were used to PCR amplify the triple promoter and red fluorescent protein from Bacillus licheniformis PP3428 genomic DNA.

Primer 065208:
(SEQ ID NO: 25)
5′-ACCTGCCTGTACACTTGCGTCCTC-3′
Primer 065209:
(SEQ ID NO: 26)
5′-CCATTTCATCCCCGCCTTACCTA-3′

The respective DNA fragment was amplified by PCR using an EXPAND® High Fidelity^{PLUS} PCR System (Roche Diagnostics, Mannheim, Germany). The PCR was composed of 1 μg of Bacillus licheniformis PP3428 genomic DNA, isolated according to the procedure described by Pitcher et al., 1989, Lett. Appl. Microbiol. 8: 151-156, 1 μl of primer 065208 (50 pmol/μl), 1 μl of primer 065209 (50 pmol/μl), 10 μl of 5×PCR buffer with 15 mM MgCl₂ (Roche Diagnostics, Mannheim, Germany), 1 μl of dNTP mix (10 mM each), 33.25 μl of water, and 0.75 μl of DNA polymerase mix (3.5 U/μl; Roche Diagnostics, Mannheim, Germany). The reaction was performed using an EPPENDORF® MASTERCYCLER® thermocycler programmed for 1 cycle at 94° C. for 2 minutes; 10 cycles each at 94° C. for 15 seconds, 58° C. for 30 seconds, and 72° C. for 1 minute and 30 seconds; and 15 cycles each at 94° C. for 15 seconds, 58° C. for 30 seconds, and 72° C. for 1 minute and 30 seconds plus a 5 second elongation at each successive cycle; 1 cycle at 72° C. for 7 minutes; and a 4° C. hold. The resulting 678 bp PCR product was isolated by 0.7% agarose gel electrophoresis using TBE buffer, excised from the gel, and purified using a QIAQUICK® Gel Extraction Kit (QIAGEN Inc., Valencia, Calif., USA) according to manufacturer's instructions.

The purified 678 bp PCR product was cloned into plasmid pCR®2.1 TOPO® (Invitrogen, Carlsbad, Calif., USA) and transformed into ONE SHOT® TOP10 chemically competent E. coli cells (Invitrogen, Carlsbad, Calif., USA) according to manufacturer's instructions, selecting for ampicillin resistance on 2×YT ampicillin plates at 37° C. Plasmid DNA was prepared from the E. coli transformants, using a QIAGEN Plasmid Midi Kit (QIAGEN Inc., Valencia, Calif., USA), and digested with Eco RI, followed by 0.7% agarose gel electrophoresis using TBE buffer. One plasmid was identified as having the correct restriction pattern and designated pBM324.

### Example 18

### Construction of a Universal Plasmid Designed to Integrate the Chloramphenicol Resistance Gene at the amyQ Locus of Bacillus amyloliquefaciens FZB24

Plasmid pBM333 was constructed in S. cerevisiae JG169 (MAT-α, ura3-52, leu2-3, pep4-1137, his3Δ2, prb1::leu2, Δpre1::his3; U.S. Pat. No. 5,770,406) using yeast recombinant-based homology cloning as follows.

The following primers were used to PCR amplify insert fragment 1, containing the 5′ region of the Bacillus amyloliquefaciens FZB24 amyQ gene, from B. amyloliquefaciens FZB24 (TAEGRO®, EPA registration number: 70127-5, EPA establishment number: 33967-NJ-1) genomic DNA, isolated according to the procedure of Pitcher et al., 1989, supra.

Primer 1202859:
(SEQ ID NO: 27)
5′-GACTCACTATAGGGAATATTAAGCTTGCTGCTATGCCGGG-3′
Primer 1202370:
(SEQ ID NO: 28)
5′-CGATTTCCAATGAGGTTAAGAGCCTAGGTGCATGAAGGATGGTCCCG
TTTTTG-3′

The following primers were used to PCR amplify insert fragment II, containing the 3′ region of the amyQ gene, from B. amyloliquefaciens FZB24 genomic DNA.

Primer 1202371:
(SEQ ID NO: 29)
5′-GATCCGAACCATTTGATCATATGTCTGACGTGTCTGCGGACAAGTT
AG-3′
Primer 1202860:
(SEQ ID NO: 30)
5′-GGCGGCCGTTACTAGTGGATCCTGCATGTTTCTCCAGCAATTG-3′

The respective DNA insert fragments I and II were amplified by PCR using an EXPAND® High Fidelity^{PLUS} PCR System. The PCR was composed of 3 μl of B. amyloliquefaciens FZB24 genomic DNA, 1 μl of primer 1202859 or primer 1202371 (50 pmol/μl), 1 μl of primer 1202370 or primer 1202860 (50 pmol/μl), 10 μl of 5×PCR buffer with 15 mM MgCl₂, 1 μl of dNTP mix (10 mM each), 33.25 μl of water, and 0.75 μl of DNA polymerase mix (3.5 U/μl). The reaction was performed using an EPPENDORF® MASTERCYCLER® thermocycler programmed for 1 cycle at 94° C. for 2 minutes; 10 cycles each at 94° C. for 15 seconds, 58° C. for 30 seconds, and 72° C. for 2 minutes; 15 cycles each at 94° C. for 15 seconds, 58° C. for 30 seconds, and 72° C. for 2 minutes plus a 5 second elongation at each successive cycle; 1 cycle at 72° C. for 7 minutes; and a 4° C. hold. The resulting approximately 2000 bp PCR product was isolated by 0.7% agarose gel electrophoresis using TBE buffer, excised from the gel, and purified using a QIAQUICK® Gel Extraction Kit according to manufacturer's instructions.

The following primers were used to PCR insert fragment III, containing the chloramphenicol resistance gene flanked by resolvase recognition sites, from Bacillus subtilis MOL2908 genomic DNA. Bacillus subtilus MOL2908 contains the chloramphenicol resistance (cat) gene isolated from pC194 (Horinouchi et al., 1982, J. Bacteriol. 150(2): 815-825) flanked by the resolvase recognition sites from pAMbeta1 (Clewell et al., 1974. J. Bacteriol. 117, 283-289).

Primer 1202369:
(SEQ ID NO: 31)
5′-CAAAAACGGGACCATCCTTCATGCACCTAGGCTCTTAACCTCATTGG
AAATCG-3′
Primer 1202372:
(SEQ ID NO: 32)
5′-CTAACTTGTCCGCAGACACGTCAGACATATGATCAAATGGTTCGGA
TC-3′

DNA insert fragment III was amplified by PCR using an EXPAND® High Fidelity^{PLUS} PCR System. The PCR was composed of 3 μl of Bacillus subtilus MOL2908 DNA, isolated according to the procedure of Pitcher et al., 1989, supra, 1 μl of primer 1202369 (50 pmol/μl), 1 μl of primer 1202372 (50 pmol/μl), 10 μl of 5×PCR buffer with 15 mM MgCl₂, 1 μl of dNTP mix (10 mM each), 33.25 μl of water, and 0.75 μl of DNA polymerase mix (3.5 U/μl). The reaction was performed using an EPPENDORF® MASTERCYCLER® thermocycler programmed for 1 cycle at 94° C. for 2 minutes; 10 cycles each at 94° C. for 15 seconds, 58° C. for 30 seconds, and 72° C. for 2 minutes; 15 cycles each at 94° C. for 15 seconds, 58° C. for 30 seconds, and 72° C. for 2 minutes plus a 5 second elongation at each successive cycle; 1 cycle at 72° C. for 7 minutes; and a 4° C. hold. The resulting 1254 bp PCR product was isolated by 0.7% agarose gel electrophoresis using TBE buffer, excised from the gel, and purified using a QIAQUICK® Gel Extraction Kit according to manufacturer's instructions.

The following primers were used to PCR amplify a plasmid fragment from plasmid pYES2 (Life Technologies, Grand Island, N.Y., USA).

Primer 1202895:
(SEQ ID NO: 33)
5′-GACTCACTATAGGGAATATTAAGCTTGCTGCTATGCCGGG-3′
Primer 1202896:
(SEQ ID NO: 34)
5′-CCCGGCATAGCAGCAAGCTTAATATTCCCTATAGTGAGTC-3′

The respective DNA plasmid fragment was amplified by PCR using an EXPAND® High Fidelity^{PLUS} PCR System. The PCR was composed of 1 μl of pYES2 (140 ng/μl), 2 μl of primer 1202895 (50 pmol/μl), 2 μl of primer 1202896 (50 pmol/μl), 5 μl of 10×PCR buffer with 15 mM MgCl₂, 1.75 μl of dNTP mix (10 mM each), 37.5 μl water, and 0.75 μl DNA polymerase mix (3.5 U/μl). The reaction was performed using an EPPENDORF® MASTERCYCLER® thermocycler programmed for 1 cycle at 94° C. for 2 minutes; 10 cycles each at 94° C. for 15 seconds, 58° C. for 30 seconds, and 72° C. for 4 minutes; 15 cycles each at 94° C. for 15 seconds, 58° C. for 30 seconds, and 72° C. for 4 minutes plus a 5 second elongation at each successive cycle; 1 cycle at 72° C. for 7 minutes; and a 4° C. hold. The resulting 7001 bp PCR product was isolated by 0.7% agarose gel electrophoresis using TBE buffer, excised from the gel, and purified using a QIAQUICK® Gel Extraction Kit according to manufacturer's instructions.

Yeast recombinant-based homology cloning in S. cerevisiae JG169 using the above mentioned fragments 1, II, and III and the plasmid fragment pYES2, was accomplished using a YEASTMAKER™ Yeast Transformation System 2 (Clontech Laboratories, Mountain View, Calif., USA) according to the manufacturer's guidelines. Yeast transformants were selected on yeast ura minus selection plates after 2 days of growth at 30° C. Several of the transformant colonies were restreaked to yeast ura minus selection plates and grown again for 2 days at 30° C. After re-isolation of the transformants on agar plates, a loopful of cells was picked and resuspended in 250 μl of P1 buffer (Qiaprep Spin Miniprep Kit; QIAGEN Inc., Valencia, Calif., USA). The cell/buffer mixture was vortexed and lysed using glass beads, after which the Kit instructions were followed according to the manufacturer's guidelines for the preparation of mini DNA. A positive clone was identified by PCR amplification of an approximately 5 kb insert fragment using primer pair 1202859 and 1202860 as follows. The EXPAND® High Fidelity Long Template PCR System (Roche Diagnostics, Mannheim, Germany) was used for the PCR amplification. The PCR was composed of 2 μl of mini DNA, 2 μl of primer 1202859 (50 pmol/μl), 2 μl of primer 1202860 (50 pmol/μl), 5 μl of 10×PCR buffer with 15 mM MgCl₂, 1.75 μl of dNTP mix (10 mM each), 36.25 μl of water, and 0.75 μl of DNA polymerase mix (3.5 U/μl). The reaction was performed using an EPPENDORF® MASTERCYCLER® thermocycler programmed for 1 cycle at 94° C. for 2 minutes; 10 cycles each at 94° C. for 15 seconds, 58° C. for 30 seconds, and 72° C. for 3 minutes; 15 cycles each at 94° C. for 15 seconds, 58° C. for 30 seconds, and 72° C. for 3 minutes plus a 5 second elongation at each successive cycle; 1 cycle at 72° C. for 7 minutes; and a 4° C. hold. The resulting approximately 5000 bp PCR product was visualized by 0.7% agarose gel electrophoresis using TBE buffer. A clone was identified as having the correct size PCR fragment and designated plasmid pBM333.

### Example 19

### Construction of Plasmid, pBM334, Designed to Integrate a Red Fluorescent Protein dsRed Gene and a Chloramphenicol Resistance Gene at the amyQ Locus of Bacillus amyloliquefaciens FZB24

Plasmid pBM334 was constructed in S. cerevisiae JG169 using yeast recombinant-based homology cloning as follows.

The following primers were used to PCR amplify the insert fragment, containing the triple promoter composed of the Bacillus licheniformis amyL promoter, short consensus B. amyloliqufaciens amyQ promoter, and B. thuringiensis cryIIIA promoter (U.S. Pat. No. 8,268,586) driving expression of the dsRED gene from plasmid pBM324.

Primer 1203544:
(SEQ ID NO: 35)
5′-GTCAAAAACGGGACCATCCTTCATGCACCTAGGACCTGCCTGTACAC
TTGCG-3′
Primer 1203545:
(SEQ ID NO: 36)
5′-GATTTCCAATGAGGTTAAGAGCCTAGGCCATTTCATCCCCGCCTTAC
CTATGC-3′

The respective DNA insert fragment was amplified by PCR using an EXPAND® High Fidelity^{PLUS} PCR System. The PCR was composed of 1 μl of 350 ng/μl pBM324, 1 μl of primer 1203544 (50 pmol/μl), 1 μl of primer 1203545 (50 pmol/μl), 10 μl of 5×PCR buffer with 15 mM MgCl₂, 1 μl of dNTP mix (10 mM each), 33.25 μl of water, and 0.75 μl of DNA polymerase mix (3.5 U/μl). The reaction was performed using an EPPENDORF® MASTERCYCLER® thermocycler programmed for 1 cycle at 94° C. for 2 minutes; 10 cycles each at 94° C. for 15 seconds, 58° C. for 30 seconds, and 72° C. for 2 minutes; 15 cycles each at 94° C. for 15 seconds, 58° C. for 30 seconds, and 72° C. for 2 minutes plus a 5 second elongation at each successive cycle; 1 cycle at 72° C. for 7 minutes; and a 4° C. hold. The resulting 2151 bp PCR product was isolated by 0.7% agarose gel electrophoresis using TBE buffer, excised from the gel, and purified using a QIAQUICK® Gel Extraction Kit according to manufacturer's instructions.

The following primers were used to PCR amplify the plasmid fragment from plasmid pBM333.

1203546:
(SEQ ID NO: 37)
5′-GCATAGGTAAGGCGGGGATGAAATGGCCTAGGCTCTTAACCTCATTG
GAAATC-3′
1203548:
(SEQ ID NO: 38)
5′-CGCAAGTGTACAGGCAGGTCCTAGGTGCATGAAGGATGGTCCCGTTT
TTGAC-3′

The respective DNA plasmid fragment was amplified by PCR using an EXPAND® High Fidelity^{PLUS} PCR System. The PCR was composed of 1 μl of pBM333 (47 ng/μl), 2 μl of primer 1203546 (50 pmol/μl), 2 μl of primer 1203548 (50 pmol/μl), 5 μl of 10×PCR buffer with 15 mM MgCl₂, 1.75 μl of dNTP mix (10 mM each), 37.25 μl water, and 0.75 μl DNA polymerase mix (3.5 U/μl). The reaction was performed using an EPPENDORF® MASTERCYCLER® thermocycler programmed for 1 cycle at 94° C. for 2 minutes; 10 cycles each at 94° C. for 15 seconds, 58° C. for 30 seconds, and 72° C. for 5 minutes; 15 cycles each at 94° C. for 15 seconds, 58° C. for 30 seconds, and 72° C. for 5 minutes plus a 5 second elongation at each successive cycle; 1 cycle at 72° C. for 7 minutes; and a 4° C. hold. The resulting 7001 bp PCR product was isolated by 0.7% agarose gel electrophoresis using TBE buffer, excised from the gel, and purified using a QIAQUICK® Gel Extraction Kit according to manufacturer's instructions.

Yeast recombinant-based homology cloning in Saccharomyces cerevisiae JG169 using the insert and plasmid fragment was accomplished using a YEASTMAKER™ Yeast Transformation System 2 according to the manufacturer's guidelines. Briefly, one colony of S. cerevisiae JG169 was used to inoculate 50 ml of YP+2% glucose medium and incubated at 30° C. overnight on an orbital shaker at 250 rpm. When the cells reached an OD₆₀₀ of 0.4 to 0.5, they were centrifuged at 1500 rpm for 5 minutes, the supernatant was discarded, and the pellet was resuspended in 30 ml of deionized water. After centrifugation at 700×g for 5 minutes in a Sorvall RT 6000D centrifuge (Thermo Fisher Scientific Inc., Raleigh, N.C., USA), the cell pellet was resuspended in 1.5 ml of 1.1×TE/LiAc solution (110 mM lithium acetate, 11 mM Tris pH 8, 1.1 mM EDTA). After centrifugation at high speed in a microcentrifuge for 15 seconds, the cell pellet was resuspended in 600 μl of 1.1× TE/LiAc solution. After addition of approximately 0.1 μg of plasmid DNA and 0.1 μg of insert DNA, 500 μl of PEG/LiAc solution (40% PEG 4000, 0.1 M lithium acetate, 10 mM Tris-HCl pH 8, 1 mM EDTA) and 5 μl of 10 mg/ml denatured Herring Testes Carrier DNA were added to 50 μl of competent cells. The mixtures were incubated at 30° C. for 30 minutes at 550 rpm with mixing by inversion every 10 minutes. A total volume of 20 μl of DMSO was added to each transformation mixture, and incubated at 42° C. for 15 minutes. The mixture was inverted every 5 minutes. The transformation mixtures were centrifuged for 15 seconds at high speed in a microcentrifuge, and the cells were resuspended in 1 ml of YPD Plus Liquid Medium (YEASTMAKER Yeast Transformation System, Clonetech, Palo Alto, Calif., USA) and incubated at 30° C. for 90 minutes at 550 rpm. After centrifugation, the cells were washed with 1 ml of 0.9% NaCl solution and resuspended in 1 ml of yeast ura minus selection medium in the presence of 15% glycerol. Fifty μl of each of the transformation reactions were spread in duplicate onto yeast ura minus selection plates.

Yeast transformants were selected on yeast ura minus selection plates after 2 days of growth at 30° C. Several of the transformant colonies were restreaked on yeast ura minus selection plates and grown again for 2 days at 30° C. After re-isolation of the transformants on agar plates, a loopful of cells was picked and resuspended in 250 μl of P1 buffer (Qiaprep Spin Miniprep Kit; QIAGEN Inc., Valencia, Calif., USA). The cell/buffer mixture was vortexed and lysed using glass beads after which the Kit instructions were followed according to the manufacturer's guidelines for the preparation of mini DNA. A positive clone was identified by PCR amplification of the 2204 bp insert fragment using primer pair 1203544 and 1203545 as follows. HERCULASE® II Fusion DNA polymerase (Stratagene, La Jolla, Calif., USA) was used for the PCR amplification. The PCR was composed of 1 μl of mini DNA, 1 μl of primer 1203544 (50 pmol/μl), 1 μl of primer 1203545 (50 pmol/μl), 10 μl of 10×PCR buffer with 15 mM MgCl₂, 1.0 μl of dNTP mix (10 mM each), 35 μl of water, and 1 μl of HERCULASE® 11 Fusion DNA polymerase. The reaction was performed using an EPPENDORF® MASTERCYCLER® thermocycler programmed for 1 cycle at 95° C. for 2 minutes; 30 cycles each at 95° C. for 20 seconds, 58° C. for 20 seconds, and 72° C. for 1 minute and 10 seconds; 1 cycle at 72° C. for 3 minutes; and a 4° C. hold. The resulting 2204 bp PCR product was visualized by 0.7% agarose gel electrophoresis using TBE buffer. A clone was identified as having the correct size PCR fragment and designated plasmid pBM334.

### Example 20

### Construction of Red Fluorescing Bacillus amyloliquefaciens FZB24 Mutant Strain, BaC0159

Bacillus amyloliquefaciens FZB24 mutant strain BaC0159 contains a red fluorescent protein dsRed gene, followed by the chloramphenicol resistance gene inserted at the amyQ gene locus. Transforming DNA, consisting of a PCR product containing the red fluorescent protein dsRed encoding gene and the chloramphenicol resistance gene flanked on both sides with the amyQ gene sequence, was prepared as follows.

The following primers were used to PCR amplify the DNA fragment from plasmid pBM334.

Primer 1202859:
(SEQ ID NO: 39)
5′-GACTCACTATAGGGAATATTAAGCTTGCTGCTATGCCGGG-3′
Primer 1202860:
(SEQ ID NO: 40)
5′-GGCGGCCGTTACTAGTGGATCCTGCATGTTTCTCCAGCAATTG-3′

The respective DNA plasmid fragment was amplified by PCR using an EXPAND® High Fidelity^{PLUS} PCR System. The PCR was composed of 1 μl of pBM334 (7.9 ng/μl), 2 μl of primer 1202859 (50 pmol/μl), 2 μl of primer 1202860 (50 pmol/μl), 5 μl of 10×PCR buffer with 15 mM MgCl₂, 1.75 μl of dNTP mix (10 mM each), 37.25 μl of water, and 0.75 μl of DNA polymerase mix (3.5 U/μl). The reaction was performed using an EPPENDORF® MASTERCYCLER® thermocycler programmed for 1 cycle at 94° C. for 2 minutes; 10 cycles each at 94° C. for 15 seconds, 58° C. for 30 seconds, and 72° C. for 4 minutes and 30 seconds; 15 cycles each at 94° C. for 15 seconds, 58° C. for 30 seconds, and 72° C. for 4 minutes and 30 seconds plus a 5 second elongation at each successive cycle; 1 cycle at 72° C. for 7 minutes; and a 4° C. hold. The resulting 7277 bp PCR product was purified using a PCR Purification Kit (QIAGEN Inc., Valencia, Calif., USA) according to manufacturer's instructions.

B. amyloliquefaciens FZB24 was spread onto LB plates to obtain single colony isolates after incubation at 37° C. overnight. After overnight incubation, one colony was used to inoculate 10 ml of LB medium, and grown in an incubator at 37° C. overnight with shaking at 250 rpm. Approximately 250 μl of the overnight culture was used to inoculate 12 ml of Spizizen I medium containing 30 μl of 1 M MgCl₂. Growth was monitored using a Klett densitometer until cells entered early stationary phase. At this point, cells were harvested, and 500 ml of the cell culture were added to a 15 ml FALCON® 2059 tube. Approximately 2.4 micrograms of transforming DNA were added to the transformation mixture and incubated at 37° C. for 30 minutes with mixing at 250 rpm. After 30 minutes, 2 μl of 50 μg/ml chloramphenicol were added to the transformation mixture. The culture was further incubated at 37° C. for an additional hour with mixing at 250 rpm, after which the cells were spread onto TBAB plus chloramphenicol plates. The plates were incubated at 37° C. until colonies appeared. A transformant with visibly red pigment and unable to clear 0.5% starch azure plates (due to the targeted integration and disruption of the amyQ gene) was isolated and designated B. amyloliquefaciens BaC0159 (red fluorescent protein-labeled TAEGRO®; RFP-TAEGRO).

### Example 21

### Xyloglucan and Vigna angularis XET16 Enhance Binding of a Biopesticide to Cellulose

Based on Examples 10, 11, 14 and 15, xyloglucan binds to plant tissue and similarly to cellulose such as filter paper. To confirm the use of xyloglucan (XG) and XET to enhance the binding of natural or biological pesticides to cellulose and hence to plants, red fluorescent protein labeled TAEGRO® (Novozymes, A/S, Bagsvaerd, Denmark) was assayed for binding to BBL® Cefinase paper discs impregnated with nitrocefin (Becton Dickinson Diagnostics, Sparks, Md., USA) to prevent contamination during incubation. In a sterile fume hood, 2 ml of red fluorescent protein-labeled TAEGRO® (RFP-TAEGRO) (Example 20) in LB medium were centrifuged and washed twice with 10 ml of sterile phosphate buffered saline (PBS). Following a final centrifugation, the RFP-TAEGRO was suspended in 2 ml total volume of PBS. Blank paper discs were incubated in triplicate in 200 μl binding reactions at ambient temperature in the dark for approximately 3 days. Binding reactions contained 20 mM sodium citrate pH 5, with or without 150 μl of RFP-TAEGRO, with or without 1 mg/ml tamarind seed xyloglucan (Megazyme, Bray, UK), and with or without 1 μM VaXET16. Following 3 days of incubation, the paper discs were washed 3 times with 1 ml of PBS and were imaged using an Epson Perfection V750 PRO (Epson America Inc., Long Beach, Calif., USA) computer scanner. Following imaging, the paper disks were used to inoculate 2 ml of sterile LB medium in a Costar #3513, 12-well cell culture cluster plate, and incubated for 12 hours at room temperature. The culture plates were imaged with an iPhone 4S cellphone camera (Apple Inc., Cupertino, Calif., USA) with the following settings: F-stop f/2.4, 1/30 s exposure time, ISO-50 and 4 mm focal length. A 100 μl aliquot of each supernatant was removed and the fluorescence was measured using a SPECTRAMAX® M5 plate reading fluorimeter (Molecular Devices, Sunnyvale, Calif., USA), with an excitation wavelength of 538 nm, and emission wavelengths from 570-650 nm.

Comparing the intensity of the red color between the variously incubated discs, discs incubated with xyloglucan, VaXET16, and RFP-TAEGRO were most intense, indicating the greatest extent of association of TAEGRO® with the discs. Discs incubated with xyloglucan and RFP-TAEGRO were less intensely colored, and those without xyloglucan were even less intense. Discs incubated only in citrate buffer show no red color. These data indicate that the amount of TAEGRO bound to cellulose was greatest in the presence of xyloglucan and VaXET16, followed by xyloglucan without VaXET16, followed by no xyloglucan.

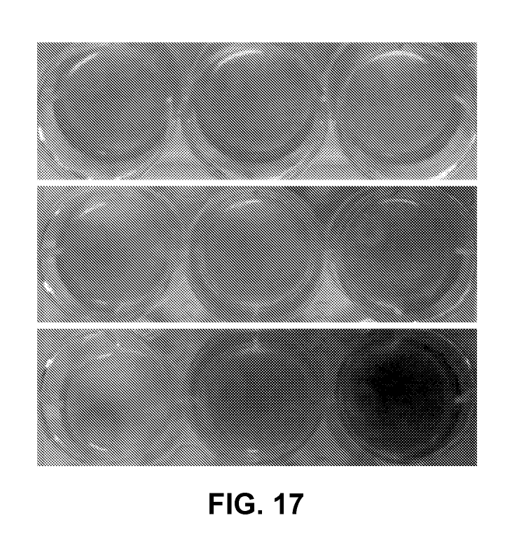

FIG. 17 shows photographs of the culture plate following a 12 hour incubation of the variously incubated discs in LB medium. Comparing the production of RFP, as indicated by red color, RFP-TAEGRO incubated with xyloglucan and VaXET16 (bottom panel) showed the most intense red color as indicated by darker suspensions in FIG. 17, whereas RFP-TAEGRO incubated with either xyloglucan (middle panel) or no xyloglucan (top panel) showed less intense red color as indicated by lighter suspensions in FIG. 17. These data indicate that a solution of xyloglucan and VaXET16 enhanced the binding of RFP-TAEGRO to cellulose.

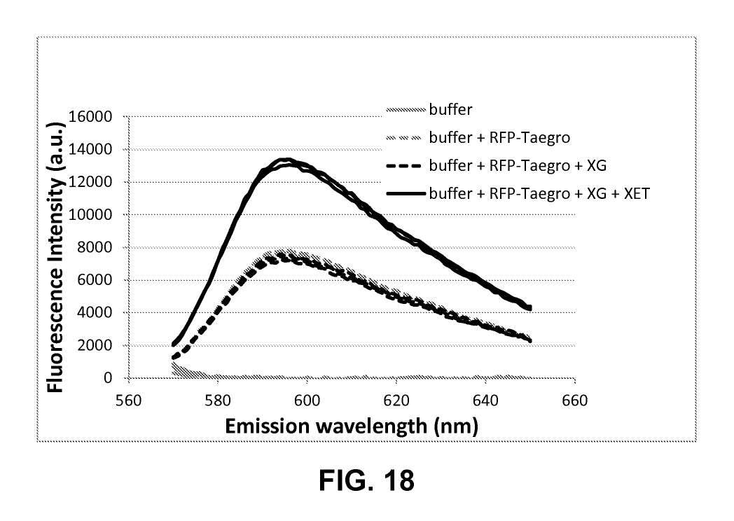

FIG. 18 shows the fluorescence spectra of LB medium inoculated with the variously incubated discs. Discs incubated in citrate buffer are shown in solid gray lines; discs incubated with RFP-TAEGRO are shown as dashed gray lines; discs incubated with RFP-TAEGRO and xyloglucan are shown as dashed black lines; and discs incubated with RFP-TAEGRO, xyloglucan, and VaXET16 are shown as solid black lines. From the spectra, the LB medium inoculated with discs incubated with RFP-TAEGRO, xyloglucan, and VaXET16 showed by far the highest fluorescence intensity. LB medium inoculated with discs incubated with RFP-TAEGRO with or without xyloglucan showed similar, intermediate fluorescence intensities. LB medium inoculated with discs incubated with citrate buffer only showed no fluorescence intensity. These data confirm that xyloglucan and VaXET16 enhanced the association of TAEGRO® with cellulose.

### Example 22

### Xyloglucan and Vigna angularis XET16 Permit Penetration of Damaged Leaves

Discs of leaves were generated as described in Example 14, with the following exceptions: rose, orange tree, and wisteria leaves were used. Leaves were assessed as “damaged” and “undamaged” by visual inspection, and damaged leaf discs were generated such that each disc had approximately equivalent surface area covered by visible blemishes. Discs were incubated with 1 mg/ml FITC-XG for 2 hours in 20 mM sodium citrate pH 5.5, with or without 1.75 μM VaXET16 in Costar #3513 12-well cell culture cluster plates with lids. Leaf discs were washed 3 times with 1 ml of deionized water.

Direct immunostaining was performed by incubating leaf pieces approximately 2-3 mm in length (cut with a razor blade from the discs previously incubated with FITC-XG) with 1 μl of Texas Red-conjugated goat polyclonal anti-fluorescein antibody (Pierce, Rockford, Ill., USA) in 100 μl of deionized H₂O in Costar #3548 48-well trays (Corning, Tewksbury, Mass., USA). Trays were wrapped in aluminum foil and were incubated for 1 hour on a rocking platform (VWR, Radnor, Pa., USA). Excess antibody was washed twice in 1 ml of phosphate-buffered saline, at pH 7.2, and left in minimal PBS buffer overnight prior to microscopy. Control incubations were performed both by incubating leaf cuttings in buffer with anti-fluorescein without prior incubation in FITC-XG, or were incubated in buffer without FITC-XG and then incubated in water without anti-fluorescein.

Laser scanning confocal microscopy was performed using an Olympus FV1000 laser scanning confocal microscope with a 10× air gap and 40× oil immersion objective lenses. Data were acquired utilizing both 488 nm and 543 nm lines of an argon ion laser excitation source in sequential acquisition mode to minimize spectral overlap of the fluorescein and Texas Red detection and to exclude chloroplast fluorescence emission. Photomultiplier tube voltage was adjusted to maximize resolution, but was kept below 800 for all images. Z-stacks were obtained by optical sectioning with 1.2 to 3 μm Z-axis step size and 3-dimensional reconstructions were generated using Olympus software.

From microscopy images, the top sides of leaves incubated with FITC-XG and Texas Red-conjugated anti-fluorescein antibody, with or without VaXET16, showed no co-localized Texas Red and FITC fluorescence emission in regions that were cuticle-covered. All co-localized emission occurred at the plasmodesma and regions between adjacent cells. The underside surfaces of leaves showed no red fluorescence emission in the cuticle, however green autofluorescence was observed. For leaves incubated without VaXET16, Texas Red emission and co-localized Texas Red/fluorescein emission were apparent at the inner surface of the stomatal guard cells, indicating that XG can access and bind to leaves at the stomata. For leaves incubated with VaXET16, co-localized fluorescence was observed at the inner surface of the stomatal guard cells, as well as below, and within the epithelial cell layer, indicating that VaXET16 improved the ability of the FITC-XG to gain access to the inside of the leaf. For damaged cells incubated with FITC-XG, Texas Red-conjugated anti-fluorescein, and VaXET16, FITC-XG and anti-fluorescein co-localization was observed within the plant leaf, though not directly adjacent to the site of the blemishes. This result indicated that the damaged spots on the leaf provided access for FITC-XG to bind to exposed cellulose within the plant tissues.

Co-localization was observed for the undamaged leaf without XET in sharply defined spots at the stomata, whereas when VaXET16 was present co-localization was spread out below the cuticle layer. In the damaged leaf, co-localization was observed much more deeply within the tissues of the leaves examined.

The present invention is further described by the following numbered paragraphs:

[1] A formulation comprising one or more (e.g., several) agriculturally beneficial agents formulated with a composition selected from the group consisting of (a) a composition comprising a xyloglucan endotransglycosylase, a polymeric xyloglucan, and a functionalized xyloglucan oligomer comprising a chemical group; (b) a composition comprising a xyloglucan endotransglycosylase, a polymeric xyloglucan functionalized with a chemical group, and a functionalized xyloglucan oligomer comprising a chemical group; (c) a composition comprising a xyloglucan endotransglycosylase, a polymeric xyloglucan functionalized with a chemical group, and a xyloglucan oligomer; (d) a composition comprising a xyloglucan endotransglycosylase, a polymeric xyloglucan, and a xyloglucan oligomer; (e) a composition comprising a xyloglucan endotransglycosylase and a polymeric xyloglucan functionalized with a chemical group; (f) a composition comprising a xyloglucan endotransglycosylase and a polymeric xyloglucan; (g) a composition comprising a xyloglucan endotransglycosylase and a functionalized xyloglucan oligomer comprising a chemical group; (h) a composition comprising a xyloglucan endotransglycosylase and a xyloglucan oligomer, and (i) a composition of (a), (b), (c), (d), (e), (f), (g), or (h) without a xyloglucan endotransglycosylase, wherein the formulation provides an agricultural benefit.

[2] The formulation of paragraph 1, wherein the one or more agriculturally beneficial agents are linked to, coated by, embedded in, or encapsulated by the polymeric xyloglucan or the polymeric xyloglucan functionalized with a chemical group.

[3] The formulation of paragraph 2, wherein the linking of the one or more agriculturally beneficial agents to the polymeric xyloglucan or the polymeric xyloglucan functionalized with a chemical group is via a covalent bond to the chemical group of the xyloglucan oligomer functionalized with the chemical group.

[4] The formulation of paragraph 2, wherein the linking of the one or more agriculturally beneficial agents to the polymeric xyloglucan is via a covalent bond to the chemical group of the polymeric xyloglucan functionalized with the chemical group.

[5] The formulation of paragraph 2, wherein the linking of the one or more agriculturally beneficial agents to the polymeric xyloglucan is via a covalent bond to the chemical group of the xyloglucan oligomer functionalized with the chemical group and a covalent bond to the chemical group of the polymeric xyloglucan functionalized with the chemical group.

[6] The formulation of paragraph 2, wherein the linking of the one or more agriculturally beneficial agents to the polymeric xyloglucan is via a covalent bond between the one or more agriculturally beneficial agents and the polymeric xyloglucan.

[7] The formulation of paragraph 2, wherein the linking of the one or more agriculturally beneficial agents to the polymeric xyloglucan is via an electrostatic interaction with the chemical group of the xyloglucan oligomer functionalized with the chemical group.

[8] The formulation of paragraph 2, wherein the linking of the one or more agriculturally beneficial agents to the polymeric xyloglucan is via an electrostatic interaction with the chemical group of the polymeric xyloglucan functionalized with the chemical group.

[9] The formulation of paragraph 2, wherein the linking of the one or more agriculturally beneficial agents to the polymeric xyloglucan is via an electrostatic interaction with the chemical group of the xyloglucan oligomer functionalized with the chemical group and an electrostatic interaction with the chemical group of the polymeric xyloglucan functionalized with the chemical group.

[10] The formulation of paragraph 2, wherein the linking of the one or more agriculturally beneficial agents to the polymeric xyloglucan is via a hydrophobic interaction with the chemical group of the xyloglucan oligomer functionalized with the chemical group.

[11] The formulation of paragraph 2, wherein the linking of the one or more agriculturally beneficial agents to the polymeric xyloglucan is via a hydrophobic interaction with the chemical group of the polymeric xyloglucan functionalized with the chemical group.

[12] The formulation of paragraph 2, wherein the linking of the one or more agriculturally beneficial agents to the polymeric xyloglucan is via a hydrophobic interaction with the chemical group of the xyloglucan oligomer functionalized with the chemical group and a hydrophobic interaction with the chemical group of the polymeric xyloglucan functionalized with the chemical group.

[13] The formulation of paragraph 2, wherein the linking of the one or more agriculturally beneficial agents to the polymeric xyloglucan is via a combination of two or more interactions selected from the group consisting of covalent, hydrophobic, and electrostatic interactions with the chemical group of the xyloglucan oligomer functionalized with the chemical group.

[14] The formulation of paragraph 2, wherein the linking of the one or more agriculturally beneficial agents to the polymeric xyloglucan is via a combination of two or more interactions selected from the group consisting of covalent, hydrophobic, and electrostatic interactions with the chemical group of the polymeric xyloglucan functionalized with the chemical group.

[15] The formulation of paragraph 2, wherein the linking of the one or more agriculturally beneficial agents to the polymeric xyloglucan is via a combination of two or more interactions selected from the group consisting of covalent, hydrophobic, and electrostatic interactions with the chemical group of the xyloglucan oligomer functionalized with the chemical group and a combination of hydrophobic and electrostatic interactions with the chemical group of the polymeric xyloglucan functionalized with the chemical group.

[16] The formulation of paragraph 2, wherein the chemical group has additional affinity or specificity for plant tissue.

[17] The formulation of any of paragraphs 1-16, wherein the one or more agriculturally beneficial agents are selected from the group consisting of fungicides, herbicides, insecticides, nematode antagonistic agents, acaricides, beneficial microorganisms, plant signal molecules, nutrients, biostimulants, preservatives, polymers, wetting agents, surfactants, anti-freezing agents, minerals, microbially stabilizing compounds, and combinations thereof.

[18] The formulation of any of paragraphs 1-17, wherein the average molecular weight of the polymeric xyloglucan ranges from 2 kDa to about 500 kDa.

[19] The formulation of any of paragraphs 1-18, wherein the average molecular weight of the xyloglucan oligomer ranges from 0.5 kDa to about 500 kDa.

[20] The formulation of any of paragraphs 1-19, wherein the xyloglucan endotransglycosylase is present at a concentration of about 0.1 nM to about 1 mM.

[21] The formulation of any of paragraphs 1-20, wherein the polymeric xyloglucan or polymeric xyloglucan functionalized with a chemical group is present at a concentration of about 1 mg to about 1 g per g of the formulation or about 0.1 μg to about 1 mg per g of the formulation.

[22] The formulation of any of paragraphs 1-20, wherein the xyloglucan oligomer or the functionalized xyloglucan oligomer is present at a concentration of about 1 mg to about 1 g per g of the formulation or about 0.1 μg to about 1 mg per g of the formulation.

[23] The formulation of any of paragraphs 1-21, wherein the xyloglucan oligomer or the functionalized xyloglucan oligomer is present with the polymeric xyloglucan at about 50:1 to about 0.5:1 molar ratio of xyloglucan oligomer or functionalized xyloglucan oligomer to polymeric xyloglucan.

[24] The formulation of any of paragraphs 1-23, wherein the xyloglucan endotransglycosylase is obtainable from a plant or microorganism.

[25] The formulation of paragraph 24, wherein the plant is selected from the group consisting of a dicotyledon and a monocotyledon.

[26] The formulation of paragraph 25, wherein the dicotyledon is selected from the group consisting of azuki beans, canola, cauliflowers, cotton, poplar or hybrid aspen, potatoes, rapes, soy beans, sunflowers, thalecress, tobacco, and tomatoes.

[27] The formulation of paragraph 25, wherein the monocotyledon is selected from the group consisting of wheat, rice, corn and sugar cane.

[28] The formulation of any of paragraphs 1-27, wherein the xyloglucan endotransglycosylase is produced by aerobic cultivation of a transformed host organism containing the appropriate genetic information from a plant.

[29] The formulation of any of paragraphs 1-28, wherein the agricultural benefit is one or more properties selected from the group consisting of improved activity of an agriculturally beneficial agent; improved adhesion to plants or plant parts; improved uptake, accessibility, or incorporation by plants; improved adhesion to soil components; increased resistance to sunlight or UV; prevention of, delay in or reduction of infestation by agricultural pests; improved resistance to run-off; reduced evaporation or volatilization; enhanced water or solvent solubility; improved uptake by plants; release caused by direct or indirect fungal or microbial activity by cellulases, hemicellulases, or accessory enzymes secreted by the microbe that degrade the polymeric xyloglucan or the cellulose with which the polymeric xyloglucan is associated; improved plant tissue-specific targeting; targeting to tissues within the plant; and improved time of release.

[30] The formulation of any of paragraphs 1-29, wherein the formulation is selected from the group consisting of an aerosol, emulsifiable concentrate, wettable powder, soluble concentrate, soluble powder, suspension concentrate, spray concentrate, capsule suspension, water dispersible granule, granules, dusts, microgranule, and seed treatment formulation.

[31] A method for enhancing plant growth, comprising applying a formulation of any of paragraphs 1-30 to a seed, a plant, a plant part, and/or a soil, comprising treating the seed, plant, plant part, or a soil.

[32] A method of formulating one or more agriculturally beneficial agents, comprising reacting the one or more (e.g., several) agriculturally beneficial agents with a composition selected from the group consisting of (a) a composition comprising a xyloglucan endotransglycosylase, a polymeric xyloglucan, and a functionalized xyloglucan oligomer comprising a chemical group; (b) a composition comprising a xyloglucan endotransglycosylase, a polymeric xyloglucan functionalized with a chemical group, and a functionalized xyloglucan oligomer comprising a chemical group; (c) a composition comprising a xyloglucan endotransglycosylase, a polymeric xyloglucan functionalized with a chemical group, and a xyloglucan oligomer; (d) a composition comprising a xyloglucan endotransglycosylase, a polymeric xyloglucan, and a xyloglucan oligomer; (e) a composition comprising a xyloglucan endotransglycosylase and a polymeric xyloglucan functionalized with a chemical group; (f) a composition comprising a xyloglucan endotransglycosylase and a polymeric xyloglucan; (g) a composition comprising a xyloglucan endotransglycosylase and a functionalized xyloglucan oligomer comprising a chemical group; (h) a composition comprising a xyloglucan endotransglycosylase and a xyloglucan oligomer, and (i) a composition of (a), (b), (c), (d), (e), (f), (g), or (h) without a xyloglucan endotransglycosylase.

[33] The method of paragraph 32, wherein the polymeric xyloglucan is functionalized with a chemical group.

[34] The method of paragraph 32, wherein the xyloglucan oligomer is functionalized with a chemical group.

[35] The method of paragraph 32, wherein both the polymeric xyloglucan and the xyloglucan oligomer are each functionalized with a chemical group.

[36] The method of any of paragraphs 32-35, wherein the one or more agriculturally beneficial agents are linked to, coated by, embedded in, or encapsulated by the polymeric xyloglucan.

[37] The method of paragraph 36, wherein the linking of the one or more agriculturally beneficial agents to the polymeric xyloglucan is via a covalent bond to the chemical group of the xyloglucan oligomer functionalized with the chemical group.

[38] The method of paragraph 36, wherein the linking of the one or more agriculturally beneficial agents to the polymeric xyloglucan is via a covalent bond to the chemical group of the polymeric xyloglucan functionalized with the chemical group.

[39] The method of paragraph 36, wherein the linking of the one or more agriculturally beneficial agents to the polymeric xyloglucan is via a covalent bond to the chemical group of the xyloglucan oligomer functionalized with the chemical group and a covalent bond to the chemical group of the polymeric xyloglucan functionalized with the chemical group.

[40] The method of paragraph 36, wherein the linking of the one or more agriculturally beneficial agents to the polymeric xyloglucan is via a covalent bond between the one or more agriculturally beneficial agents and the polymeric xyloglucan.

[41] The method of paragraph 36, wherein the linking of the one or more agriculturally beneficial agents to the polymeric xyloglucan is via an electrostatic interaction with the chemical group of the xyloglucan oligomer functionalized with the chemical group.

[42] The method of paragraph 36, wherein the linking of the one or more agriculturally beneficial agents to the polymeric xyloglucan is via an electrostatic interaction with the chemical group of the polymeric xyloglucan functionalized with the chemical group.

[43] The method of paragraph 36, wherein the linking of the one or more agriculturally beneficial agents to the polymeric xyloglucan is via an electrostatic interaction with the chemical group of the xyloglucan oligomer functionalized with the chemical group and an electrostatic interaction with the chemical group of the polymeric xyloglucan functionalized with the chemical group.

[44] The method of paragraph 36, wherein the linking of the one or more agriculturally beneficial agents to the polymeric xyloglucan is via a hydrophobic interaction with the chemical group of the xyloglucan oligomer functionalized with the chemical group.

[45] The method of paragraph 36, wherein the linking of the one or more agriculturally beneficial agents to the polymeric xyloglucan is via a hydrophobic interaction with the chemical group of the polymeric xyloglucan functionalized with the chemical group.

[46] The method of paragraph 36, wherein the linking of the one or more agriculturally beneficial agents to the polymeric xyloglucan is via a hydrophobic interaction with the chemical group of the xyloglucan oligomer functionalized with the chemical group and a hydrophobic interaction with the chemical group of the polymeric xyloglucan functionalized with the chemical group.

[47] The method of paragraph 36, wherein the linking of the one or more agriculturally beneficial agents to the polymeric xyloglucan is via a combination of two or more interactions selected from the group consisting of covalent, hydrophobic, and electrostatic interactions with the chemical group of the xyloglucan oligomer functionalized with the chemical group.

[48] The method of paragraph 36, wherein the linking of the one or more agriculturally beneficial agents to the polymeric xyloglucan is via a combination of two or more interactions selected from the group consisting of covalent, hydrophobic, and electrostatic interactions with the chemical group of the polymeric xyloglucan functionalized with the chemical group.

[49] The method of paragraph 36, wherein the linking of the one or more agriculturally beneficial agents to the polymeric xyloglucan is via a combination of two or more interactions selected from the group consisting of covalent, hydrophobic, and electrostatic interactions with the chemical group of the xyloglucan oligomer functionalized with the chemical group and a combination of hydrophobic and electrostatic interactions with the chemical group of the polymeric xyloglucan functionalized with the chemical group.

[50] The method of paragraph 36, wherein the chemical group has additional affinity or specificity for plant tissue.

[51] The method of any of paragraphs 32-50, wherein the one or more agriculturally beneficial agents are selected from the group consisting of fungicides, herbicides, insecticides, nematode antagonistic agents, acaricides, beneficial microorganisms, plant signal molecules, nutrients, biostimulants, preservatives, polymers, wetting agents, surfactants, anti-freezing agents, minerals, microbially stabilizing compounds, and combinations thereof.

[52] The method of any of paragraphs 32-51, wherein the average molecular weight of the polymeric xyloglucan ranges from 2 kDa to about 500 kDa.

[53] The method of any of paragraphs 32-52, wherein the average molecular weight of the xyloglucan oligomer ranges from 0.5 kDa to about 500 kDa.

[54] The method of any of paragraphs 32-53, wherein the xyloglucan endotransglycosylase is preferably present at about 0.1 nM to about 1 mM.

[55] The method of any of paragraphs 32-54, wherein the polymeric xyloglucan or polymeric xyloglucan functionalized with a chemical group is present at a concentration of about 1 mg to about 1 g or about 0.1 μg to about 1 mg per g of the agriculturally beneficial agent.

[56] The method of any of paragraphs 32-55, wherein the xyloglucan oligomer or the functionalized xyloglucan oligomer is present at a concentration of about 1 mg to about 1 g or about 0.1 μg to about 1 mg per g of the agriculturally beneficial agent.

[57] The method of any of paragraphs 32-56, wherein the xyloglucan oligomer or the functionalized xyloglucan oligomer is present with the polymeric xyloglucan at about 50:1 to about 0.5:1 molar ratio of xyloglucan oligomer or functionalized xyloglucan oligomer to polymeric xyloglucan.

[58] The method of any of paragraphs 32-57, wherein the xyloglucan endotransglycosylase is obtainable from a plant or microorganism.

[59] The method of paragraph 58, wherein the plant is selected from the group consisting of a dicotyledon and a monocotyledon.

[60] The method of paragraph 59, wherein the dicotyledon is selected from the group consisting of cauliflowers, soy beans, azuki beans, tomatoes, potatoes, rapes, sunflowers, cotton, tobacco, poplar, aspen, and hybrid aspen.

[61] The method of paragraph 59, wherein the monocotyledon is selected from the group consisting of wheat, rice, corn and sugar cane.

[62] The method of any of paragraphs 32-61, wherein the xyloglucan endotransglycosylase is produced by aerobic cultivation of a transformed host organism containing the appropriate genetic information from a plant.

[63] The method of any of paragraphs 32-62, wherein the formulation is selected from the group consisting of an aerosol, emulsifiable concentrate, wettable powder, soluble concentrate, soluble powder, suspension concentrate, spray concentrate, capsule suspension, water dispersible granule, granules, dusts, microgranule, and seed treatment formulation.

The inventions described and claimed herein are not to be limited in scope by the specific aspects herein disclosed, since these aspects are intended as illustrations of several aspects of the invention. Any equivalent aspects are intended to be within the scope of the inventions. Indeed, various modifications of the inventions in addition to those shown and described herein will become apparent to those skilled in the art from the foregoing description. Such modifications are also intended to fall within the scope of the appended claims. In the case of conflict, the present disclosure including definitions will control.

1. A formulation comprising one or more agriculturally beneficial agents formulated with a composition selected from the group consisting of (a) a composition comprising a xyloglucan endotransglycosylase, a polymeric xyloglucan, and a functionalized xyloglucan oligomer comprising a chemical group; (b) a composition comprising a xyloglucan endotransglycosylase, a polymeric xyloglucan functionalized with a chemical group, and a functionalized xyloglucan oligomer comprising a chemical group; (c) a composition comprising a xyloglucan endotransglycosylase, a polymeric xyloglucan functionalized with a chemical group, and a xyloglucan oligomer; (d) a composition comprising a xyloglucan endotransglycosylase, a polymeric xyloglucan, and a xyloglucan oligomer; (e) a composition comprising a xyloglucan endotransglycosylase and a polymeric xyloglucan functionalized with a chemical group; (f) a composition comprising a xyloglucan endotransglycosylase and a polymeric xyloglucan; (g) a composition comprising a xyloglucan endotransglycosylase and a functionalized xyloglucan oligomer comprising a chemical group; (h) a composition comprising a xyloglucan endotransglycosylase and a xyloglucan oligomer, and (i) a composition of (a), (b), (c), (d), (e), (f), (g), or (h) without a xyloglucan endotransglycosylase, wherein the formulation provides an agricultural benefit.

2. The formulation of claim 1, wherein the one or more agriculturally beneficial agents are linked to, coated by, embedded in, or encapsulated by the polymeric xyloglucan or the polymeric xyloglucan functionalized with a chemical group.

3. The formulation of claim 2, wherein the linking of the one or more agriculturally beneficial agents to the polymeric xyloglucan or the polymeric xyloglucan functionalized with a chemical group is via a covalent bond to the chemical group of the xyloglucan oligomer functionalized with the chemical group; wherein the linking of the one or more agriculturally beneficial agents to the polymeric xyloglucan is via a covalent bond to the chemical group of the polymeric xyloglucan functionalized with the chemical group; wherein the linking of the one or more agriculturally beneficial agents to the polymeric xyloglucan is via a covalent bond to the chemical group of the xyloglucan oligomer functionalized with the chemical group and a covalent bond to the chemical group of the polymeric xyloglucan functionalized with the chemical group; wherein the linking of the one or more agriculturally beneficial agents to the polymeric xyloglucan is via a covalent bond between the one or more agriculturally beneficial agents and the polymeric xyloglucan; wherein the linking of the one or more agriculturally beneficial agents to the polymeric xyloglucan is via an electrostatic interaction with the chemical group of the xyloglucan oligomer functionalized with the chemical group; wherein the linking of the one or more agriculturally beneficial agents to the polymeric xyloglucan is via an electrostatic interaction with the chemical group of the polymeric xyloglucan functionalized with the chemical group; wherein the linking of the one or more agriculturally beneficial agents to the polymeric xyloglucan is via an electrostatic interaction with the chemical group of the xyloglucan oligomer functionalized with the chemical group and an electrostatic interaction with the chemical group of the polymeric xyloglucan functionalized with the chemical group; wherein the linking of the one or more agriculturally beneficial agents to the polymeric xyloglucan is via a hydrophobic interaction with the chemical group of the xyloglucan oligomer functionalized with the chemical group; wherein the linking of the one or more agriculturally beneficial agents to the polymeric xyloglucan is via a hydrophobic interaction with the chemical group of the polymeric xyloglucan functionalized with the chemical group; wherein the linking of the one or more agriculturally beneficial agents to the polymeric xyloglucan is via a hydrophobic interaction with the chemical group of the xyloglucan oligomer functionalized with the chemical group and a hydrophobic interaction with the chemical group of the polymeric xyloglucan functionalized with the chemical group; wherein the linking of the one or more agriculturally beneficial agents to the polymeric xyloglucan is via a combination of two or more interactions selected from the group consisting of covalent, hydrophobic, and electrostatic interactions with the chemical group of the xyloglucan oligomer functionalized with the chemical group; wherein the linking of the one or more agriculturally beneficial agents to the polymeric xyloglucan is via a combination of two or more interactions selected from the group consisting of covalent, hydrophobic, and electrostatic interactions with the chemical group of the polymeric xyloglucan functionalized with the chemical group; and wherein the linking of the one or more agriculturally beneficial agents to the polymeric xyloglucan is via a combination of two or more interactions selected from the group consisting of covalent, hydrophobic, and electrostatic interactions with the chemical group of the xyloglucan oligomer functionalized with the chemical group and a combination of hydrophobic and electrostatic interactions with the chemical group of the polymeric xyloglucan functionalized with the chemical group.

4. The formulation of claim 1, wherein the one or more agriculturally beneficial agents are selected from the group consisting of fungicides, herbicides, insecticides, nematode antagonistic agents, acaricides, beneficial microorganisms, plant signal molecules, nutrients, biostimulants, preservatives, polymers, wetting agents, surfactants, anti-freezing agents, minerals, microbially stabilizing compounds, and combinations thereof.

5. The formulation of claim 1, wherein the average molecular weight of the polymeric xyloglucan ranges from 2 kDa to about 500 kDa.

6. The formulation of claim 1, wherein the average molecular weight of the xyloglucan oligomer ranges from 0.5 kDa to about 500 kDa.

7. The formulation of claim 1, wherein the xyloglucan endotransglycosylase is present at a concentration of about 0.1 nM to about 1 mM.

8. The formulation of claim 1, wherein the polymeric xyloglucan or polymeric xyloglucan functionalized with a chemical group is present at a concentration of about 1 mg to about 1 g per g of the formulation or about 0.1 μg to about 1 mg per g of the formulation.

9. The formulation of claim 1, wherein the xyloglucan oligomer or the functionalized xyloglucan oligomer is present at a concentration of about 1 mg to about 1 g per g of the formulation or about 0.1 μg to about 1 mg per g of the formulation.

10. The formulation of claim 1, wherein the xyloglucan oligomer or the functionalized xyloglucan oligomer is present with the polymeric xyloglucan at about 50:1 to about 0.5:1 molar ratio of xyloglucan oligomer or functionalized xyloglucan oligomer to polymeric xyloglucan.

11. (canceled)

12. The formulation of claim 1, wherein the formulation is selected from the group consisting of an aerosol, emulsifiable concentrate, wettable powder, soluble concentrate, soluble powder, suspension concentrate, spray concentrate, capsule suspension, water dispersible granule, granules, dusts, microgranule, and seed treatment formulation.

13. A method for enhancing plant growth, comprising applying a formulation of claim 1 to a seed, a plant, a plant part, and/or a soil, comprising treating the seed, plant, plant part, or a soil.

14. A method of formulating one or more agriculturally beneficial agents, comprising reacting the one or more (e.g., several) agriculturally beneficial agents with a composition selected from the group consisting of (a) a composition comprising a xyloglucan endotransglycosylase, a polymeric xyloglucan, and a functionalized xyloglucan oligomer comprising a chemical group; (b) a composition comprising a xyloglucan endotransglycosylase, a polymeric xyloglucan functionalized with a chemical group, and a functionalized xyloglucan oligomer comprising a chemical group; (c) a composition comprising a xyloglucan endotransglycosylase, a polymeric xyloglucan functionalized with a chemical group, and a xyloglucan oligomer; (d) a composition comprising a xyloglucan endotransglycosylase, a polymeric xyloglucan, and a xyloglucan oligomer; (e) a composition comprising a xyloglucan endotransglycosylase and a polymeric xyloglucan functionalized with a chemical group; (f) a composition comprising a xyloglucan endotransglycosylase and a polymeric xyloglucan; (g) a composition comprising a xyloglucan endotransglycosylase and a functionalized xyloglucan oligomer comprising a chemical group; (h) a composition comprising a xyloglucan endotransglycosylase and a xyloglucan oligomer, and (i) a composition of (a), (b), (c), (d), (e), (f), (g), or (h) without a xyloglucan endotransglycosylase.

15-25. (canceled)
